(19) United States
(12) Patent Application Publication
(10) Pub. No.: US 2025/0013148 A1
(43) Pub. Date: Jan. 9, 2025

(54) RESIST COMPOSITION, RESIST PATTERN FORMING METHOD, COMPOUND, AND ACID GENERATOR

(71) Applicant: TOKYO OHKA KOGYO CO., LTD., Kawasaki-shi (JP)

(72) Inventors: Takuya Uehara, Kawasaki-shi (JP); KhanhTin Nguyen, Kawasaki-shi (JP); Takuya Ikeda, Kawasaki-shi (JP)

(21) Appl. No.: 18/691,733

(22) PCT Filed: Sep. 20, 2022
(86) PCT No.: PCT/JP2022/034949

§ 371 Date: Mar. 13, 2024

(30) Foreign Application Priority Data
Sep. 24, 2021 (JP) 2021-155752

(51) Int. Cl. G03F 7/004; C07C 309/12; G03F 7/00; G03F 7/039; G03F 7/30

(52) U.S. Cl. CPC G03F 7/0045; C07C 309/12; G03F 7/0007; G03F 7/0397; G03F 7/30; G03F 7/70033

(57) ABSTRACT

A resist composition including a base material component and a compound represented by General Formula (b0), in which Rb⁰ represents a fused cyclic group in which an aromatic ring and an alicyclic ring are fused, the alicyclic ring in the fused cyclic group has substituents, at least one of the substituents contains a hydrocarbon group having a bromine atom or an iodine atom, Yb⁰ represents a divalent linking group or a single bond, Yb⁰ is bonded to the alicyclic ring in the fused cyclic group, Vb⁰ represents a single bond, an alkylene group, or a fluorinated alkylene group, R⁰ represents a fluorinated alkyl group having 1 to 5 carbon atoms or a fluorine atom, M^{m+} represents an m-valent organic cation, and m represents an integer of 1 or greater

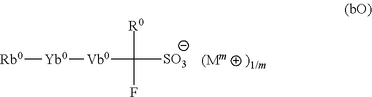

### TECHNICAL FIELD

The present invention relates to a resist composition, a resist pattern forming method, a compound, and an acid generator.

Priority is claimed on Japanese Patent Application No. 2021-155752, filed Sep. 24, 2021, the content of which is incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization technologies typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed due to an action of an acid and an acid generator component that generates an acid upon light exposure has been used in the related art.

In the resist pattern formation, the behavior of an acid generated from an acid generator component upon light exposure is considered as one factor that has a great influence on lithography characteristics.

As the acid generator that is used in the chemically amplified resist composition, a wide variety of acid generators have been suggested in the related art. Known examples of the acid generator include onium salt-based acid generators such as an iodonium salt and a sulfonium salt, oxime sulfonate-based acid generators, diazomethane-based acid generators, nitrobenzyl sulfonate-based acid generators, iminosulfonate-based acid generators, and disulfonate-based acid generators.

As the onium salt-based acid generators, those having an onium ion such as triphenylsulfonium in a cation moiety are mainly used. An alkyl sulfonate ion or a fluorinated alkyl sulfonate ion in which some or all hydrogen atoms of the alkyl group have been substituted with fluorine atoms is typically used in an anion moiety of the onium salt-based acid generator.

Further, in order to improve the lithography characteristics in the resist pattern formation, an onium salt-based acid generator having an anion with a specific structure as an anion moiety of the onium salt-based acid generator has also been suggested (for example, in Patent Document 1).

### CITATION LIST

### Patent Document

### [Patent Document 1]

Japanese Unexamined Patent Application, First Publication No. 2018-92159

### SUMMARY OF INVENTION

### Technical Problem

With the further progress of the lithography technologies and resist pattern miniaturization, for example, the goal in lithography using extreme ultraviolet rays (EUV) or electron beams (EB) is to form a minute pattern with a size of several tens of nanometers. As the dimensions of a resist pattern decrease as described above, a resist composition from which a resist pattern having satisfactory critical dimension uniformity (CDU) of pattern dimensions can be formed while having high sensitivity to an exposure light source is required.

Further, since particularly EUV and EB among exposure light sources have a smaller number of photons related to photosensitivity as compared with an ArF excimer laser and a KrF excimer laser, the sensitivity of the resist composition is required to be further improved.

However, in such a resist composition containing an onium salt-based acid generator described in Patent Document 1 as described above, since the anion moiety has a polycyclic structure having a bicyclooctane skeleton, the hydrophobicity is improved, and thus the uniformity of the onium salt-based acid generator in the resist film can be increased. However, there is room for further improvement in sensitivity.

The present invention has been made in consideration of the above-described circumstances, and an object of the present invention is to provide a resist composition capable of achieving high sensitivity, from which a resist pattern having satisfactory CDU can be formed, a resist pattern forming method using the resist composition, a novel compound useful as an acid generator of the resist composition, and an acid generator using the compound.

### Solution to Problem

In order to achieve the above-described object, the present invention employs the following configurations.

That is, according to a first aspect of the present invention, there is provided a resist composition which generates an acid upon light exposure and whose solubility in a developing solution is changed due to an action of the acid, the resist composition including: a base material component (A) whose solubility in a developing solution is changed due to an action of an acid and an acid generator component (B) which generates an acid upon light exposure, in which the acid generator component (B) contains a compound (B0) represented by General Formula (b0).

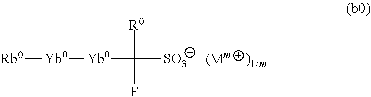

[In the formula, Rb⁰ represents a fused cyclic group in which an aromatic ring and an alicyclic ring are fused. The alicyclic ring in the fused cyclic group has substituents, and at least one of the substituents contains a hydrocarbon group having a bromine atom or a hydrocarbon group having an iodine atom. Yb⁰ represents a divalent linking group or a single bond. Here, Yb⁰ is bonded to the alicyclic ring in the fused cyclic group. Vb⁰ represents a single bond, an alkylene group, or a fluorinated alkylene group. R⁰ represents a fluorinated alkyl group having 1 to 5 carbon atoms or a fluorine atom. M^{m+} represents an m-valent organic cation. m represents an integer of 1 or greater.]

According to a second aspect of the present invention, there is provided a resist pattern forming method including a step of forming a resist film on a support using the resist composition according to the first aspect, a step of exposing the resist film to light, and a step of developing the resist film exposed to light to form a resist pattern.

According to a third aspect of the present invention, there is provided a compound represented by General Formula (b0).

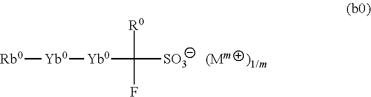

[In the formula, Rb⁰ represents a fused cyclic group in which an aromatic ring and an alicyclic ring are fused. The alicyclic ring in the fused cyclic group has substituents, and at least one of the substituents contains a hydrocarbon group having an iodine atom. Yb⁰ represents a divalent linking group or a single bond. Here, Yb⁰ is bonded to the alicyclic ring in the fused cyclic group. Vb⁰ represents a single bond, an alkylene group, or a fluorinated alkylene group. R⁰ represents a fluorinated alkyl group having 1 to 5 carbon atoms or a fluorine atom. M^{m+} represents an m-valent organic cation. m represents an integer of 1 or greater.]

According to a fourth aspect of the present invention, there is provided an acid generator containing the compound according to the third aspect described above.

### Advantageous Effects of Invention

According to the present invention, it is possible to provide a resist composition capable of achieving high sensitivity, from which a resist pattern having satisfactory CDU can be formed, a resist pattern forming method using the resist composition, a novel compound useful as an acid generator of the resist composition, and an acid generator using the compound.

### DESCRIPTION OF EMBODIMENTS

In the present specification and the scope of the present claims, the term “aliphatic” is a relative concept used in relation to the term “aromatic”, and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “light exposure” is a general concept for irradiation with radiation.

The term “acid decomposable group” indicates a group having acid decomposability in which at least a part of a bond in the structure of the acid decomposable group can be cleaved due to the action of an acid.

Examples of the acid decomposable group whose polarity is increased due to the action of an acid include groups which are decomposed due to the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved due to the action of an acid and a group (ii) in which some bonds are cleaved due to the action of an acid, a decarboxylation reaction occurs, and thus the bond between the acid dissociable group and the atom adjacent to the acid dissociable group can be cleaved.

It is necessary that the acid dissociable group that constitutes the acid decomposable group be a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, those having a molecular weight of 500 or greater and less than 4000 are typically used. Hereinafter, the term “low-molecular-weight compound” denotes a non-polymer having a molecular weight of 500 or greater and less than 4000. As the polymer, those having a molecular weight of 1000 or greater are typically used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the a-position may be substituted with a substituent. The substituent (R^{+x}) that substitutes the hydrogen atom bonded to the carbon atom at the a-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed due to an action of the acid.

Such a resist composition contains a base material component (A) (hereinafter, also referred to as “component (A)”) whose solubility in a developing solution is changed due to the action of an acid, and an acid generator component (B) that generates an acid upon light exposure (hereinafter, also referred to as “component (B)”).

In a case where a resist film is formed using the resist composition of the present embodiment and the formed resist film is subjected to selective light exposure, an acid is generated from the component (B) at an exposed portion of the resist film, and the solubility of the component (A) in a developing solution is not changed at an unexposed portion of the resist film while the solubility of the component (A) in a developing solution is changed by the action of the acid, and thus a difference in solubility in a developing solution occurs between the exposed portion and the unexposed portion. Therefore, in a case where the resist film is developed, the exposed portion of the resist film is dissolved and removed to form a positive-tone resist pattern in a case where the resist composition is of a positive tone, whereas the unexposed portion of the resist film is dissolved and removed to form a negative-tone resist pattern in a case where the resist composition is of a negative tone.

In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition. The resist composition of the present embodiment may be a positive-tone resist composition or a negative-tone resist composition. Further, the resist composition of the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Component (A)>

In the resist composition of the present embodiment, it is preferable that the component (A) have a resin component (A1) whose solubility in a developing solution is changed due to the action of an acid (hereinafter, also referred to as “component (A1)”). In the alkali developing process and the solvent developing process, since the polarity of the base material component before and after the light exposure is changed by using the component (A1), an excellent development contrast can be obtained.

As the component (A), another polymer compound and/or a low-molecular-weight compound may be used in combination with the component (A1).

In the resist composition according to the present embodiment, the component (A) may be used alone or in a combination of two or more kinds thereof.

In the resist composition according to the present embodiment, the component (A) may be “base material component which generates an acid upon light exposure and whose solubility in a developing solution is changed due to an action of the acid”. In a case where the component (A) is a base material component which generates an acid upon light exposure and whose solubility in a developing solution is changed due to an action of an acid, it is preferable that a component (A1) described below be a resin which generates an acid upon light exposure and whose solubility in a developing solution is changed due to an action of an acid. As such a resin, a polymer compound having a constitutional unit that generates an acid upon light exposure can be used. As the constitutional unit that generates an acid upon light exposure, those which are known can be used.

### In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable.

Further, the component (A1) may have other constitutional units as necessary in addition to the constitutional unit (a1).

<<Constitutional Unit (a1)>>

The constitutional unit (a1) is a constitutional unit that contains an acid decomposable group whose polarity is increased due to the action of an acid.

Examples of the acid dissociable group are the same as those which have been suggested as the acid dissociable groups of the base resin for a chemically amplified resist composition.

Specific examples of the acid dissociable group of the base resin proposed for a chemically amplified resist composition include “acetal type acid dissociable group” and “tertiary alkyl ester type acid dissociable group”, “secondary alkyl ester type acid dissociable group”, and “tertiary alkyloxycarbonyl acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

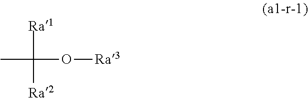

[In the formula, Ra′¹ and Ra′² each represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represent a hydrogen atom and more preferable that both Ra′¹ and Ra′² represent a hydrogen atom.

In a case where Ra′¹ or Ra′² represents an alkyl group, examples of the alkyl group include the same alkyl groups exemplified as the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of the same kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group. Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6] decanyl group, a tricyclo[3.3.1.13,7] decanyl group, a tetracyclo[6.2.1.13,6.02,7] dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′¹ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

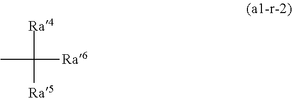

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ independently represent a hydrocarbon group without being bonded to one another, suitable examples thereof include a group represented by General Formula (a1-r2-4).

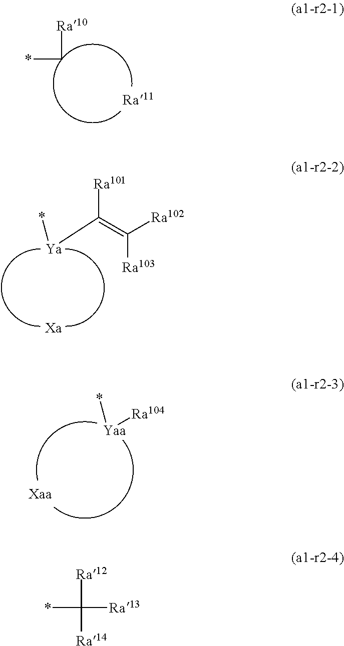

[In Formula (a1-r2-1), Ra′¹⁰ represents a linear or branched alkyl group having 1 to 12 carbon atoms, some of which may be substituted with a halogen atom or a hetero atom-containing group. Ra′¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra′¹⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms. Some or all hydrogen atoms in this chain-like saturated hydrocarbon group may be substituted. Ra′¹⁴ represents a hydrocarbon group which may have a substituent. * represents a bonding site.]

In Formula (a1-r2-1), Ra′¹⁰ represents a linear or branched alkyl group having 1 to 12 carbon atoms, some of which may be substituted with a halogen atom or a hetero atom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a hetero atom-containing group. For example, some hydrogen atoms constituting the alkyl group may be substituted with a halogen atom or a hetero atom-containing group. Further, some carbon atoms (methylene group or the like) constituting the alkyl group may be substituted with a hetero atom-containing group.

Examples of the hetero atoms here include an oxygen atom, a nitrogen atom, and a sulfur atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹ (an aliphatic cyclic group that is formed together with a carbon atom to which Ra′¹⁰ is bonded) include the groups exemplified as the aliphatic hydrocarbon group (alicyclic hydrocarbon group) which is a monocyclic group or a polycyclic group as Ra′³ in Formula (a1-r-1). Among them, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-r-1).

The cyclic hydrocarbon group that is formed by Xa together with Ya may have a substituent. Examples of the substituent include those exemplified as the substituents that the cyclic hydrocarbon group as Ra′³ may have.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6]decanyl group, a tricyclo[3.3.1.13,7]decanyl group, a tetracyclo[6.2.1.13,6.02,7] dodecanyl group, or an adamantyl group. From the viewpoint of ease of synthesis, Ra¹⁰¹ to Ra¹⁰³ each represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same substituents as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group exemplified as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms. Examples of the chain-like monovalent saturated hydrocarbon group having 1 to 10 carbon atoms as Ra′² and Ra′¹³ include those exemplified as the chain-like monovalent saturated hydrocarbon group having 1 to 10 carbon atoms as Ra¹⁰¹ to Ra¹⁰³. Some or all hydrogen atoms in this chain-like saturated hydrocarbon group may be substituted.

Ra′¹² and Ra′¹³ each represent preferably a hydrogen atom or an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same substituents as those for Ra^{x}.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra¹⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

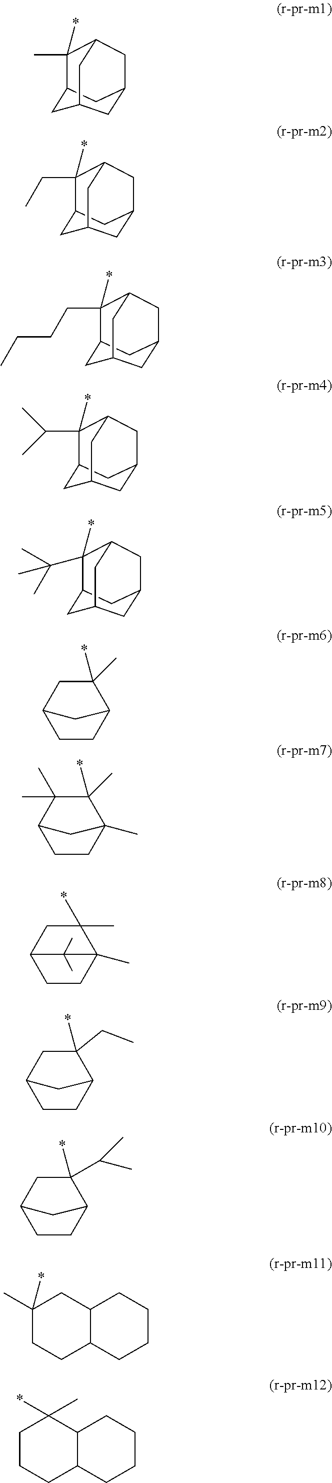

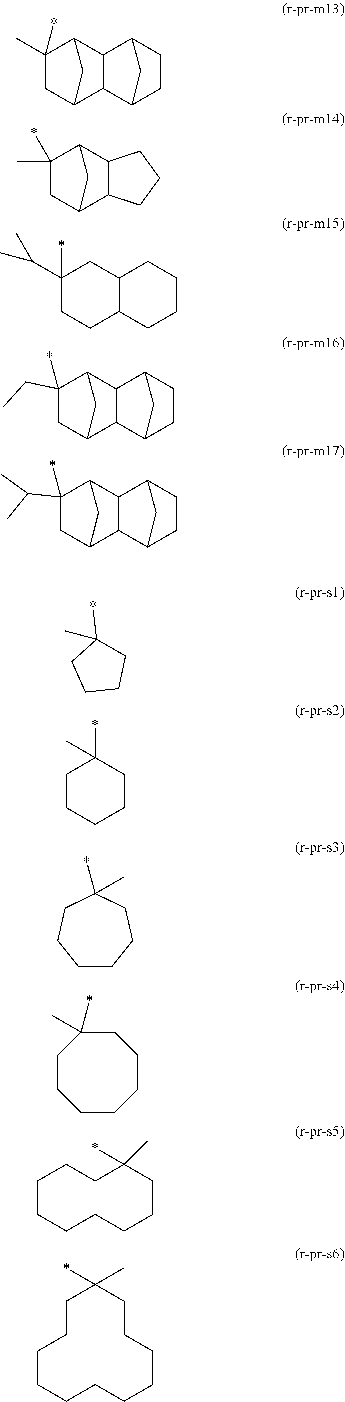

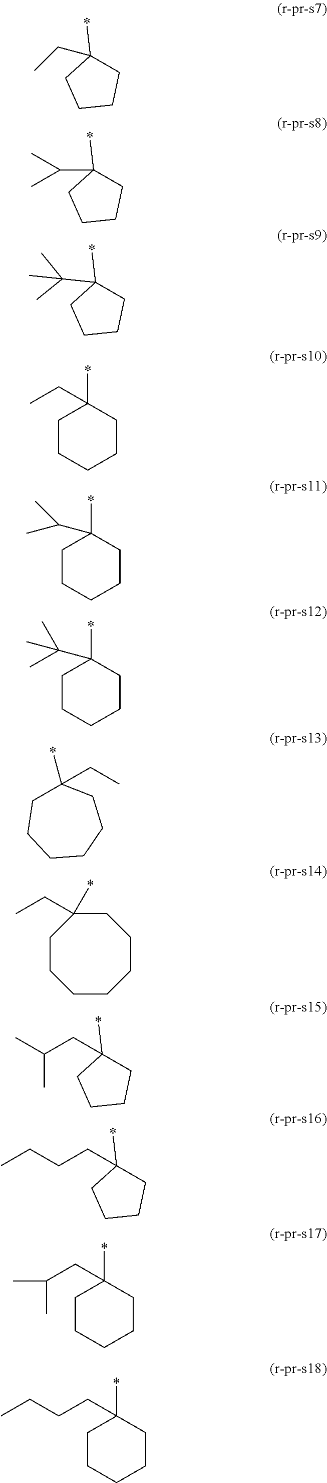

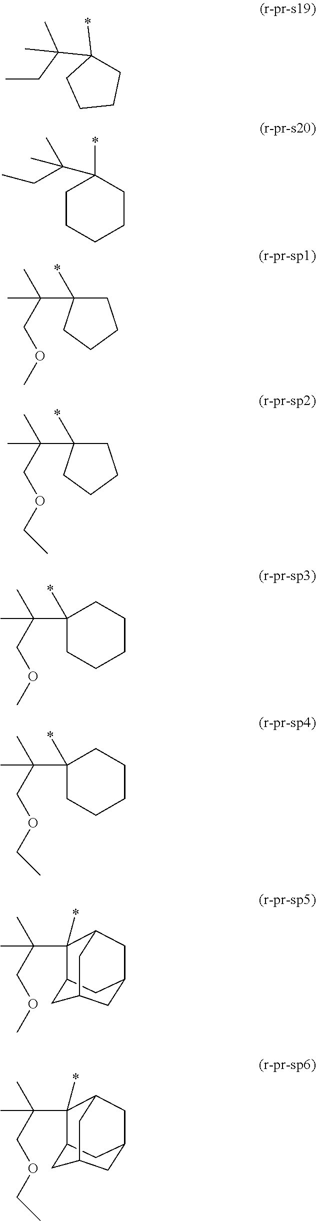

Specific examples of the group represented by Formula (a1-r2-2) are shown below.

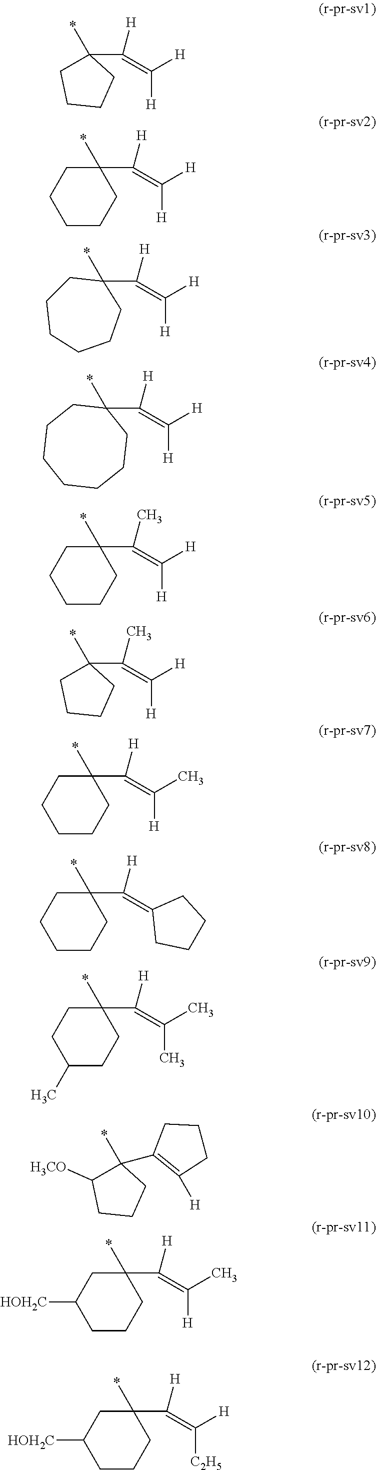

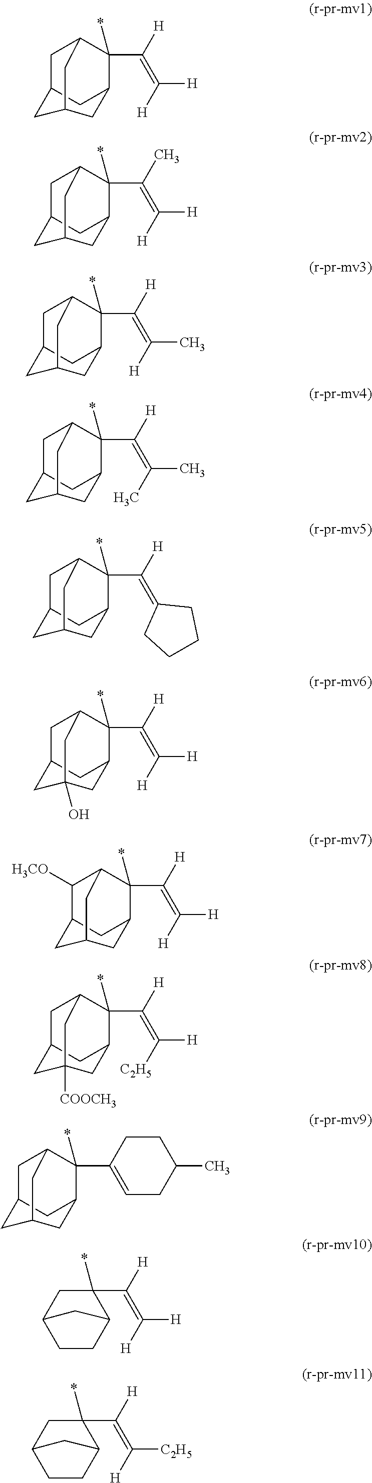

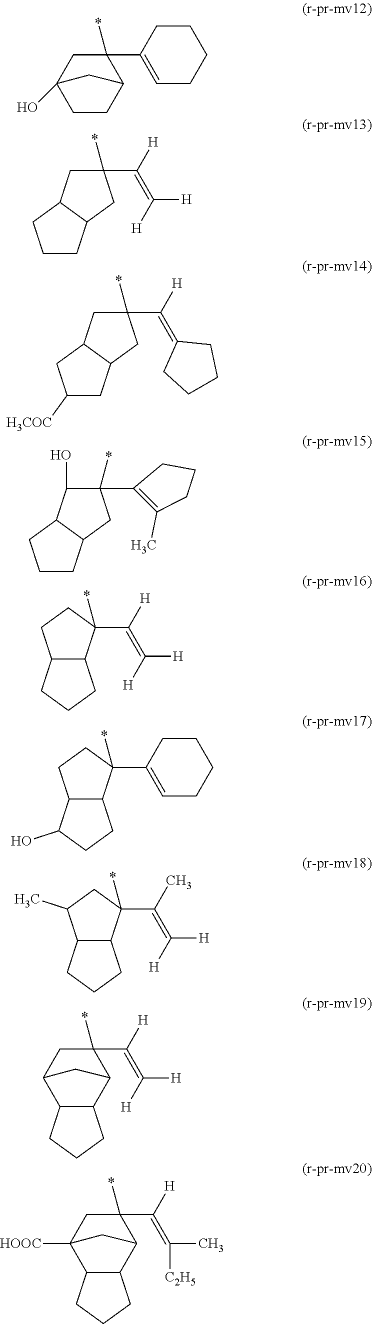

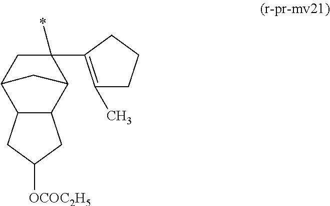

Specific examples of the group represented by Formula (a1-r2-3) are shown below.

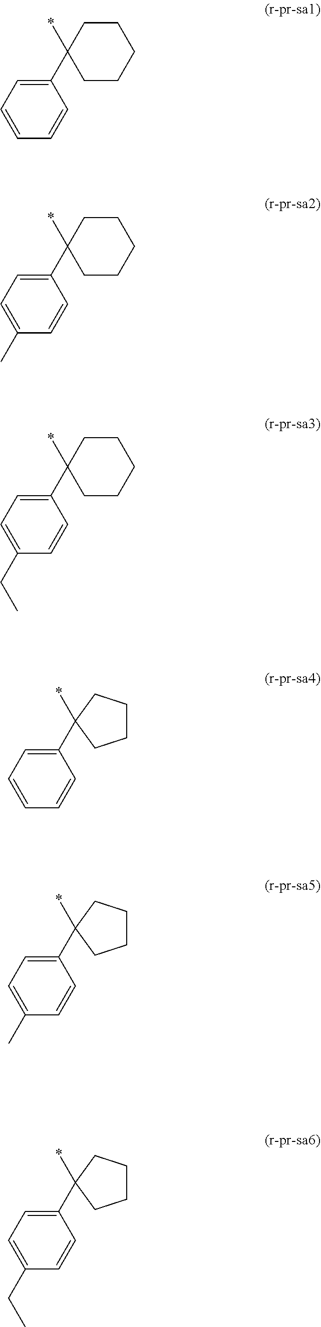 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

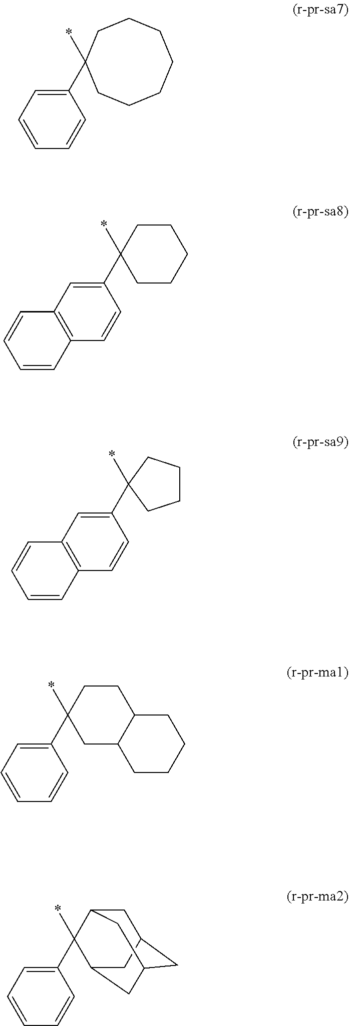 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

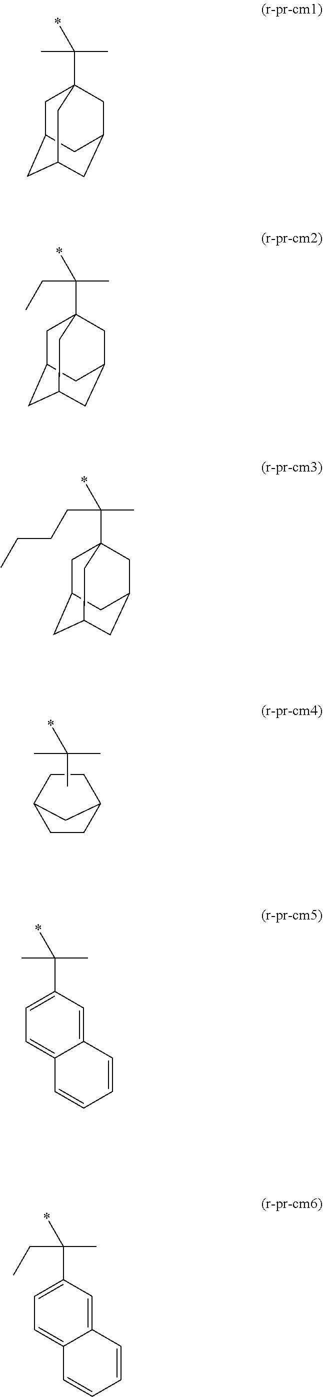

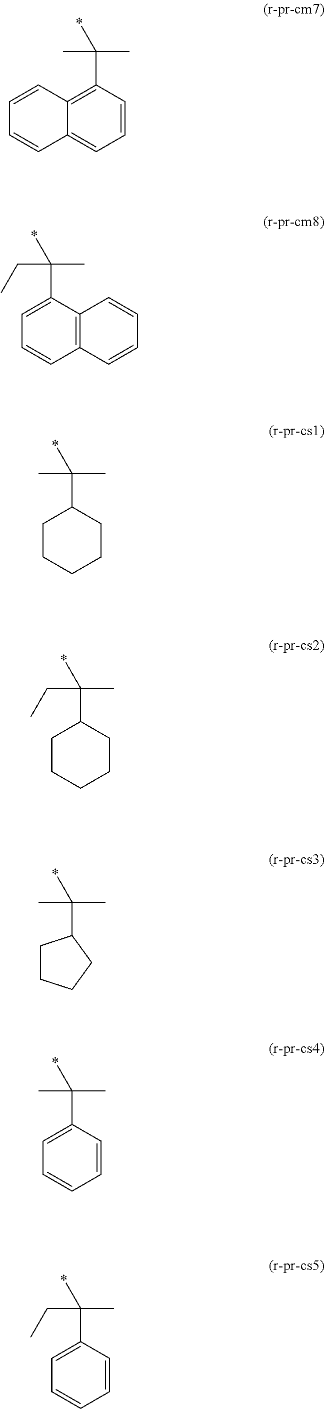

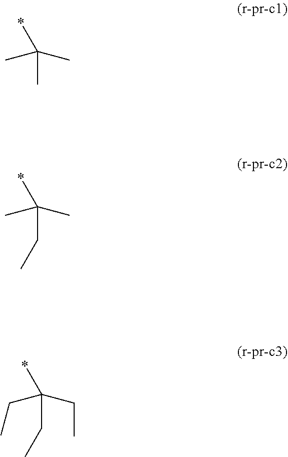

Secondary alkyl ester type acid dissociable group:

Examples of the acid dissociable group that protects a carboxy group among the polar groups include an acid dissociable group represented by General Formula (a1-r-4).

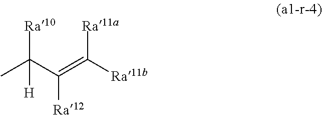

[In the formula, Ra′¹⁰ represents a hydrocarbon group. Ra′¹¹a and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of the substituent include Ra^{x5} described above.

Ra′¹⁰ and Ra′11a or Ra′^{11b} may be bonded to each other to form a ring. The ring may be a polycyclic ring or a monocyclic ring, and may be an alicyclic ring or an aromatic ring.

The alicyclic ring and the aromatic ring may have a hetero atom.

Among the examples described above, as the ring formed by Ra′¹⁰ and Ra′^{11a} or Ra′^{11b} being bonded to each other, a monocycloalkene, a ring in which some carbon atoms of a monocycloalkene have been substituted with hetero atoms (such as an oxygen atom and a sulfur atom), or a monocycloalkadiene is preferable, a cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a fused ring. Specific examples of the fused ring include indane.

The ring formed by Ra′¹⁰ and Ra′^{11a} or Ra′^{11b} being bonded to each other may have a substituent. Examples of the substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

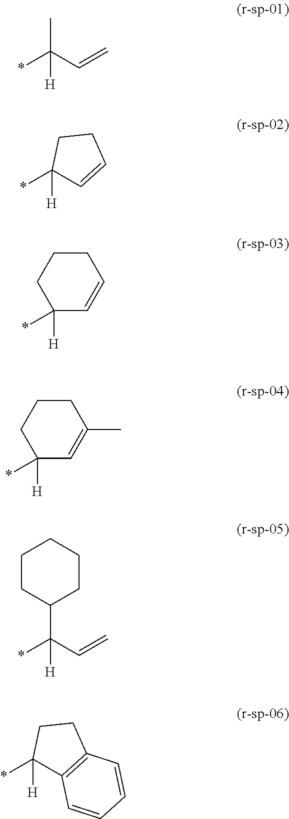

Tertiary alkyloxycarbonyl acid dissociable group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

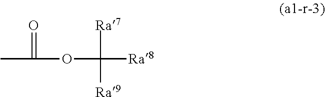

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having l to 3 carbon atoms.

Further, the total number of carbon atoms in each alkyl group is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

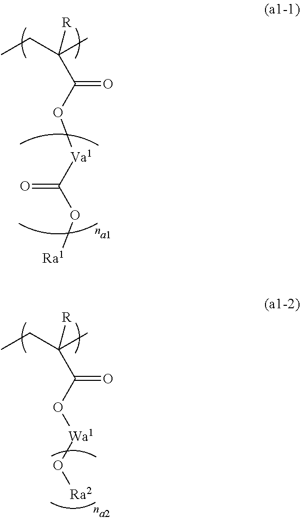

[In the formulae, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid dissociable group represented by General Formula (a1-r-1) or (a1-r-2). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3).]

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Val may be saturated or unsaturated. In general, it is preferable that the aliphatic hydrocarbon group be saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a I-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by Formula (a1-r-1) or (a1-r-2).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group be saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In the Formula (a1-2), Ra² represents an acid dissociable group represented by General Formula (a1-r-1) or (a1-r-3).

Specific examples of the constitutional unit represented by Formula (a1-1) are shown below. In each of the formulae shown below, R^{α} represents a hydrogen atom, a methyl group, or a trifluoromethyl group.

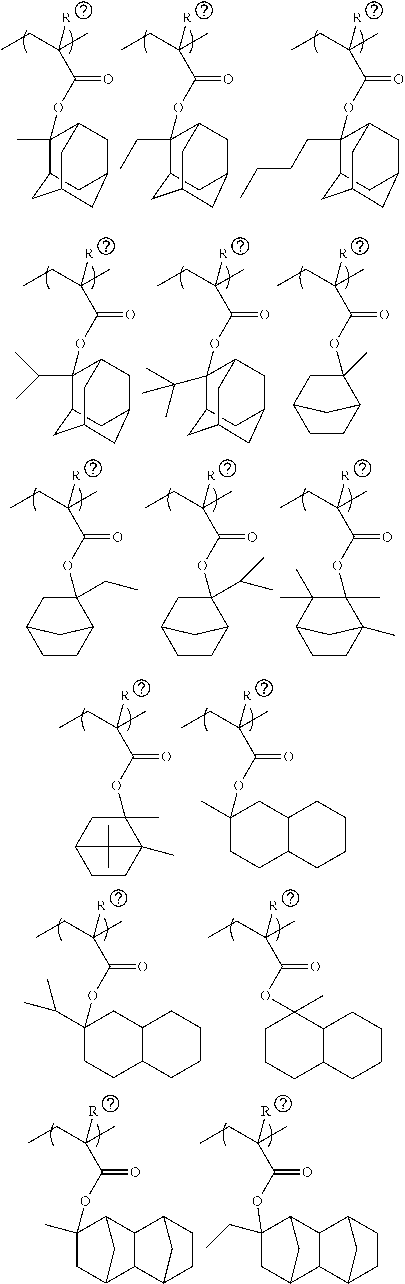

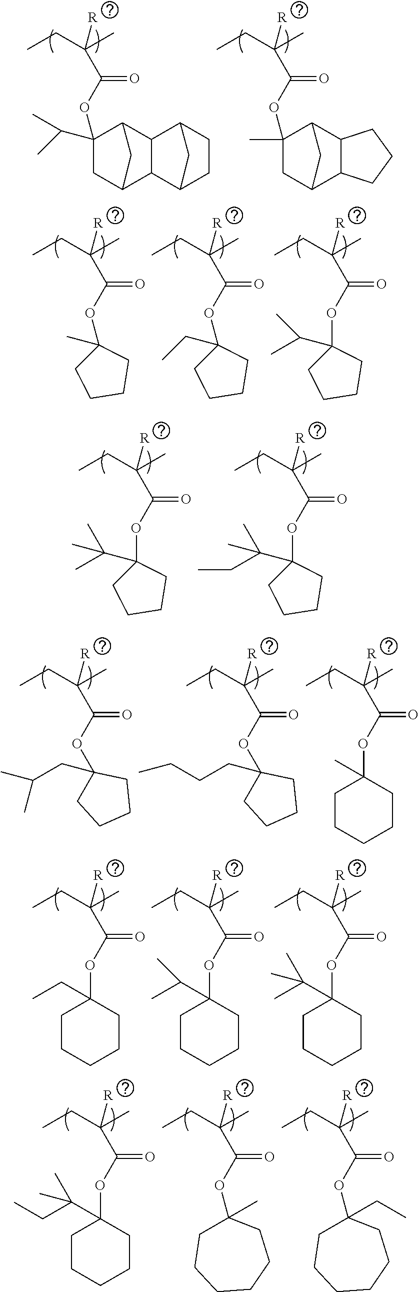

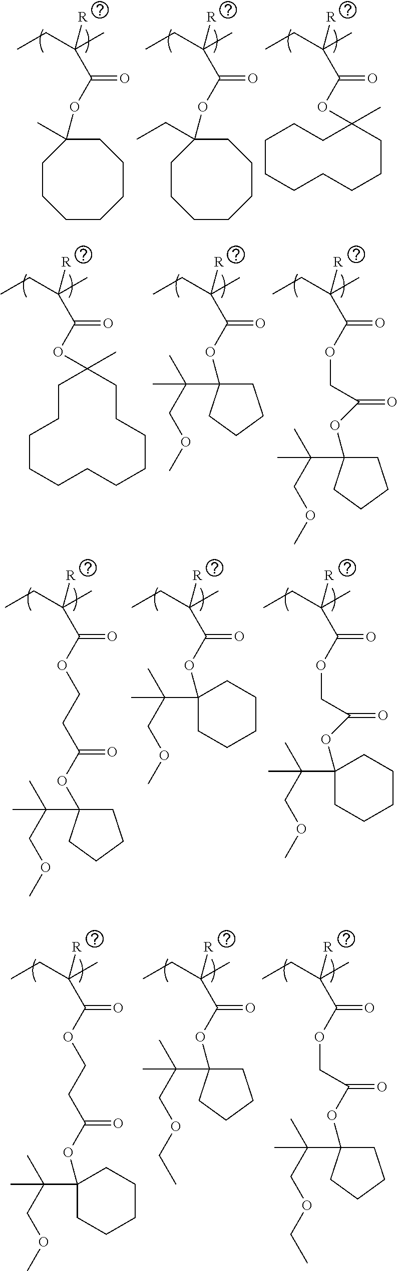

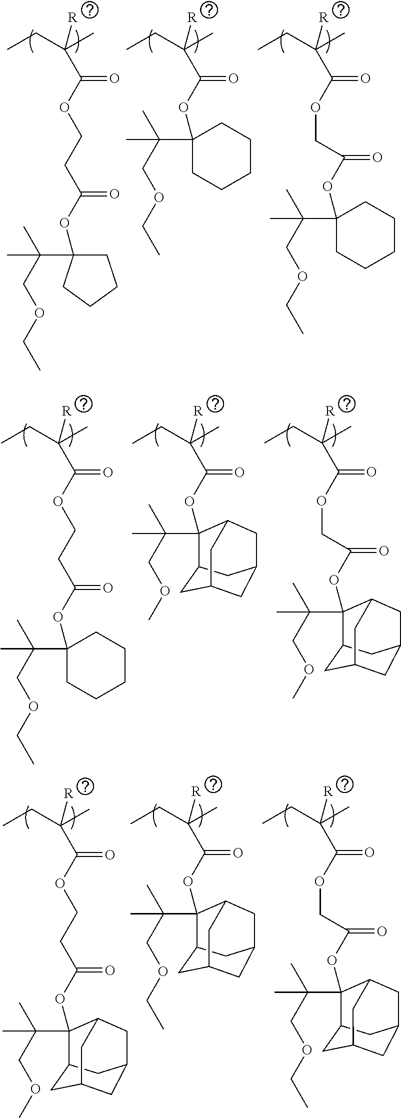

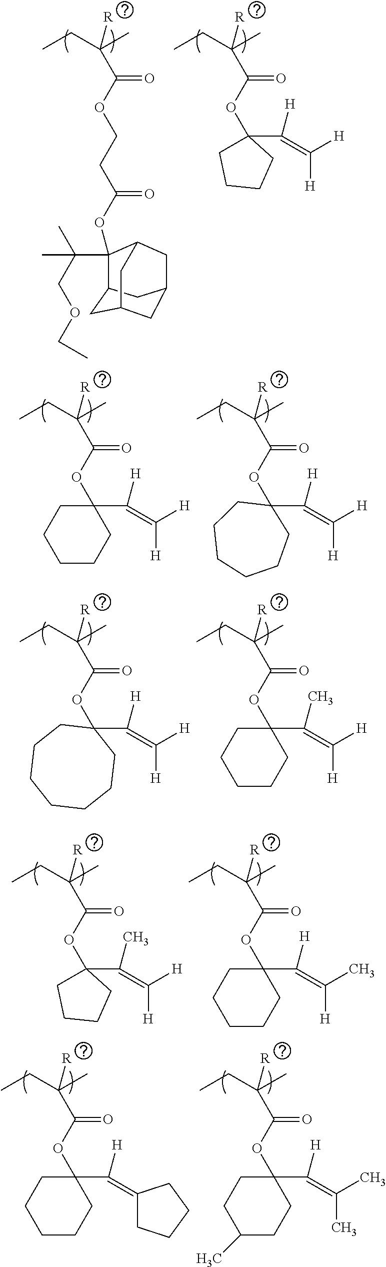

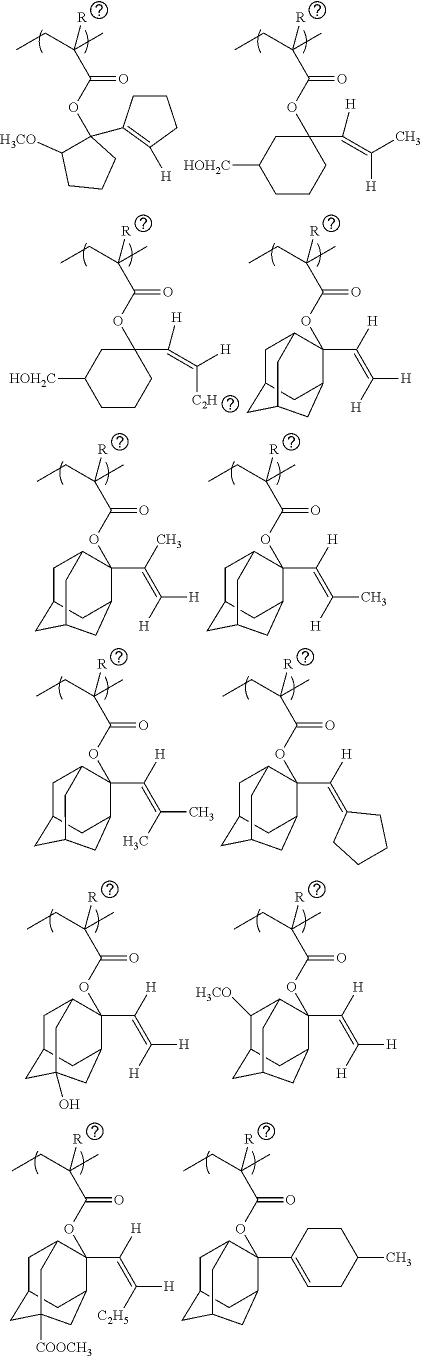

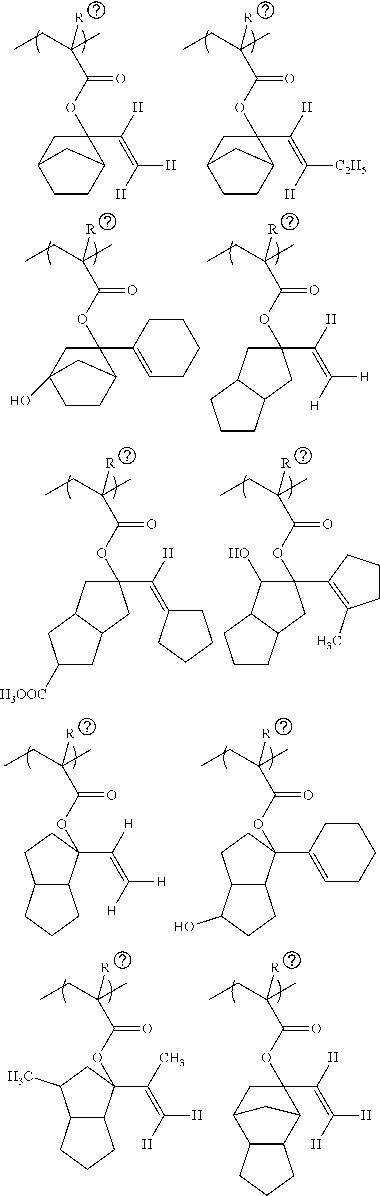

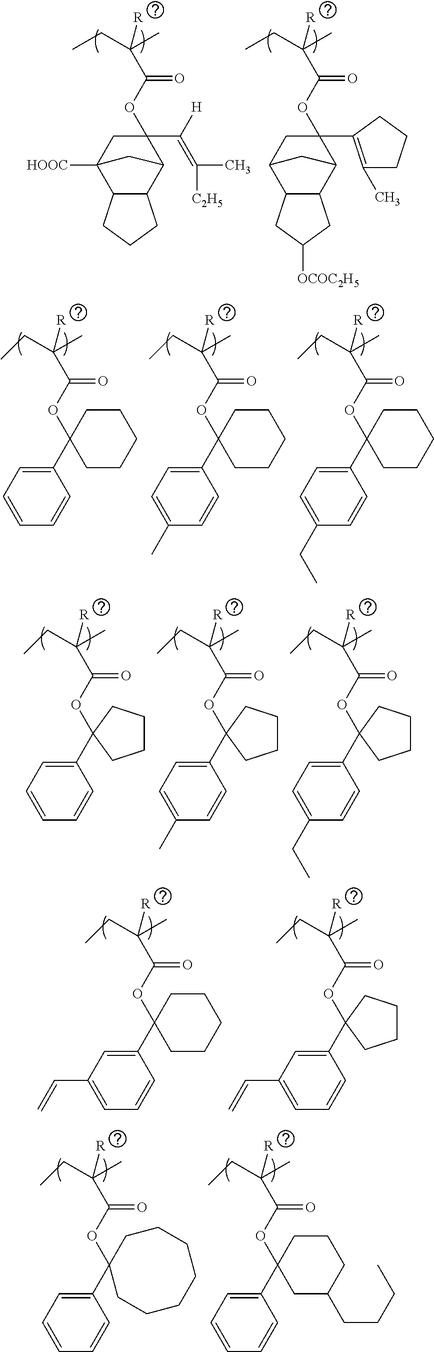

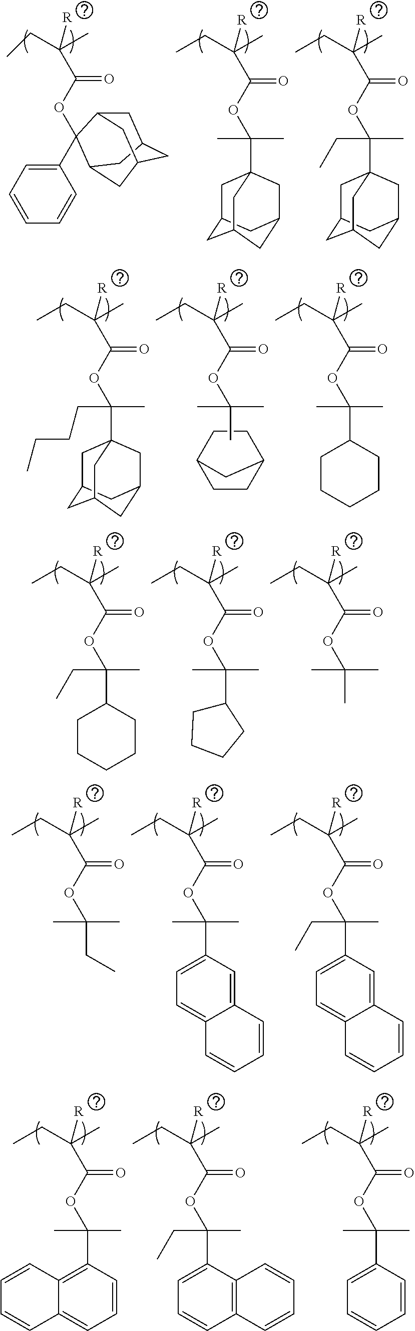

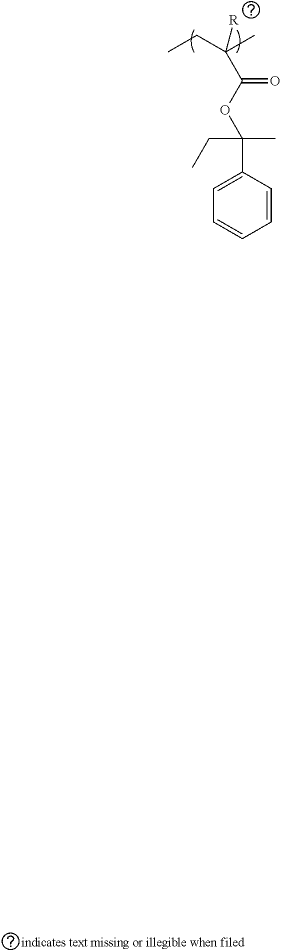

The constitutional unit (a1) included in the component (A 1) may be used alone or two or more kinds thereof may be used.

Since the lithography characteristics (the sensitivity, the shape, and the like) are easily improved using electron beams or EUV, a constitutional unit represented by Formula (a1-1) is preferable as the constitutional unit (a1).

Among the examples, as the constitutional unit (a1), those having a constitutional unit represented by General Formula (a1-1-1) are particularly preferable.

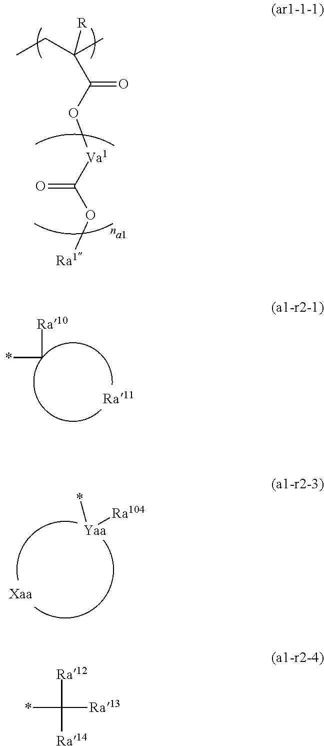

[In the formulae, Ra¹″ represents an acid dissociable group represented by General Formula (a1-r2-1), (a1-r2-3), or (a1-r2-4). * represents a bonding site.]

R, Va¹, and nₐ₁ in Formula (a1-1-1) have the same definitions as R, Va¹, and nₐ₁ in Formula (a1-1), respectively.

The description of the acid dissociable group represented by General Formula (a1-r2-1), (a1-r2-3), or (a1-r2-4) is the same as described above. Among these, it is preferable to select those in which the acid dissociable group is a cyclic group because the reactivity is enhanced for EB or EUV, which is preferable.

In Formula (a1-1-1), it is preferable that Ra¹″ represents an acid dissociable group represented by General Formula (a1-r2-1) among the examples described above.

The proportion of the constitutional unit (a1) in the component (A1) is preferably in a range of 5% to 95% by mole, more preferably in a range of 10% to 90% by mole, still more preferably in a range of 30% to 70% by mole, and particularly preferably in a range of 40% to 60% by mole, with respect to the total amount (100% by mole) of all constitutional units constituting the component (A1).

In a case where the proportion of the constitutional unit (a1) is set to be greater than or equal to the lower limits of the above-described preferable ranges, lithography characteristics such as the sensitivity, CDU, the resolution, and reduction of the roughness are improved. Further, in a case where the proportion of the constitutional unit (a1) is less than or equal to the upper limits of the above-described preferable ranges, the constitutional unit (a1) and other constitutional units can be balanced, and the lithography characteristics are improved.

### <<Other Constitutional Units>>

Further, the component (A1) may have other constitutional units as necessary in addition to the constitutional unit (a1).

Examples of the other constitutional units include a constitutional unit (a10) represented by General Formula (a10-1), a constitutional unit (a2) containing a lactone-containing cyclic group, a constitutional unit (a8) derived from a compound represented by General Formula (a8-1), and a constitutional unit (a01) derived from a compound represented by General Formula (a0-1).

In regard to constitutional unit (a10):

The constitutional unit (a10) is a constitutional unit represented by General Formula (a10-1).

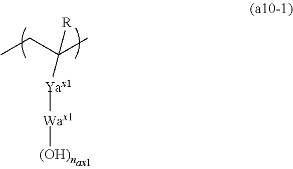

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In the Formula (a10-1), R represents a hydrogen atom, an alkyl group having 1 to 5 carbon atoms, or a halogenated alkyl group having 1 to 5 carbon atoms.

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or a trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a hetero atom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as the aromatic ring is a cyclic conjugated system having (4n+2) n electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Further, examples of the aromatic hydrocarbon group as Wa^{x1} also include a group in which (nₐₓ₁+1) hydrogen atoms have been removed from an aromatic compound having an aromatic ring (for example, biphenyl or fluorene) which may have two or more substituents.

Among the examples, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those exemplified as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} have no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In each of the formulae shown below, R^{α} represents a hydrogen atom, a methyl group, or a trifluoromethyl group.

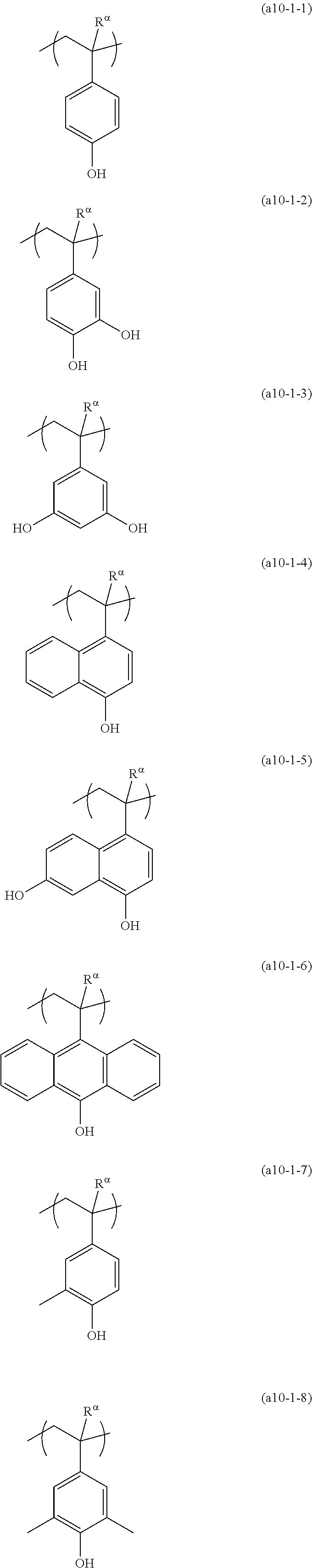

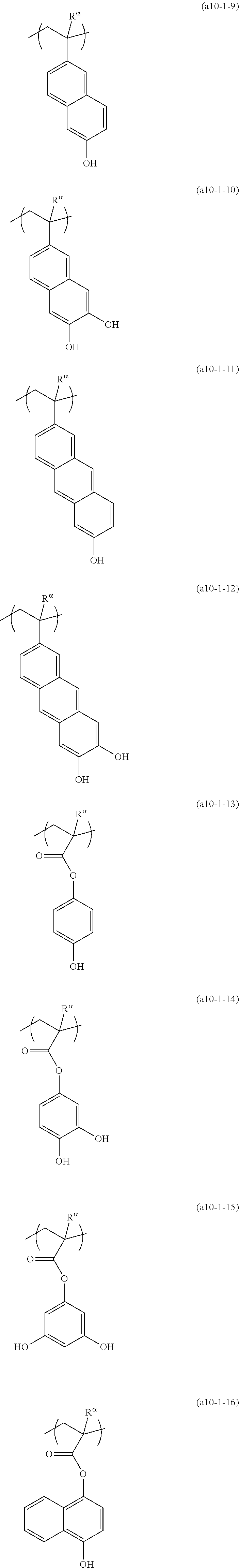

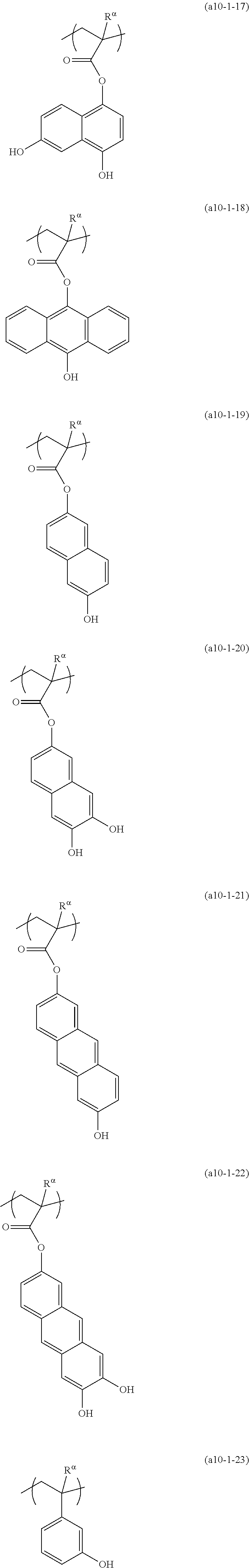

The constitutional unit (a10) included in the component (A1) may be used alone or two or more kinds thereof may be used.

In a case where the component (A1) has the constitutional unit (a10), the proportion of the constitutional unit (a10) in the component (A l) is preferably in a range of 5% to 95% by mole, more preferably in a range of 10% to 90% by mole, still more preferably in a range of 30% to 70% by mole, and particularly preferably in a range of 40% to 60% by mole, with respect to the total amount (100% by mole) of all constitutional units constituting the component (A1).

In a case where the proportion of the constitutional unit (a10) is greater than or equal to the lower limits, the sensitivity can be more easily increased. On the other hand, in a case where the proportion thereof is set to be less than or equal to the upper limits, the constitutional unit (a10) and other constitutional units are likely to be balanced.

In regard to constitutional unit (a2):

The component (A1) may further have a constitutional unit (a2) (here, a constitutional unit corresponding to the constitutional unit (a1) is excluded) containing a lactone-containing cyclic group, in addition to the constitutional unit (a1).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups represented by each of General Formulae (a2-r-1) to (a2-r-7).

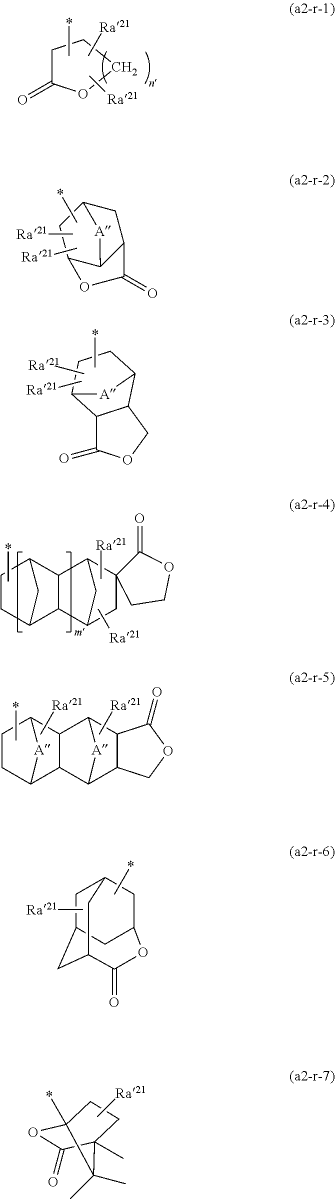

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ is 0 or 1. * represents a bonding site.]

In General Formulae (a2-r-1) to (a2-r-7), it is preferable that the alkyl group as Ra′²¹ be an alkyl group having 1 to 6 carbon atom. Further, it is preferable that the alkyl group be linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ be an alkoxy group having 1 to 6 carbon atoms. Further, it is preferable that the alkoxy group be linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group at the same time.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as a bicycloalkane, a tricycloalkane, or a tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups represented by each of General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups represented by each of General Formulae (a2-r-1) to (a2-r-7) are shown below.

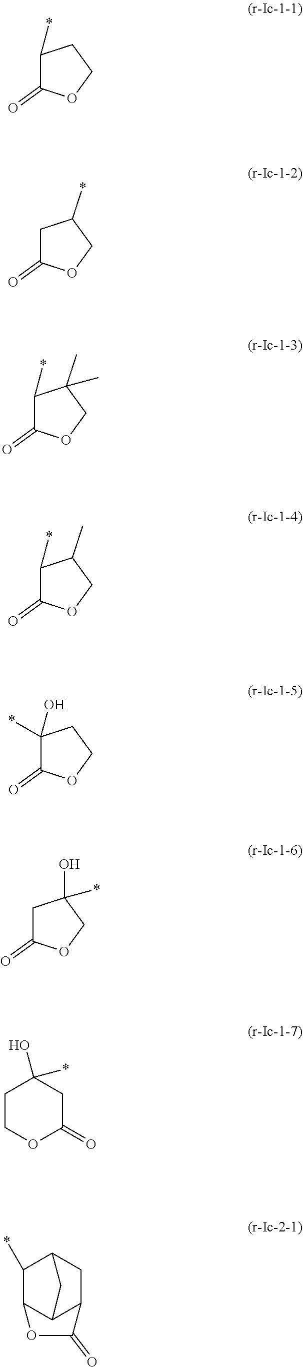

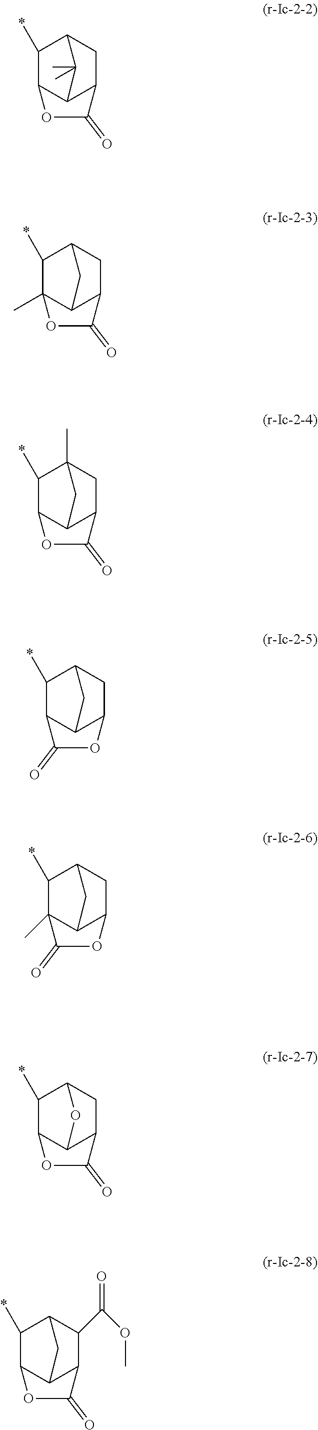

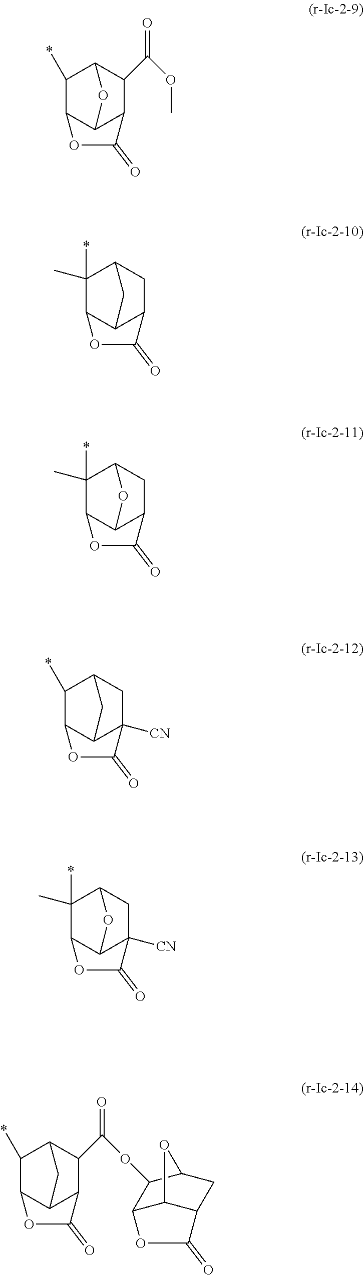

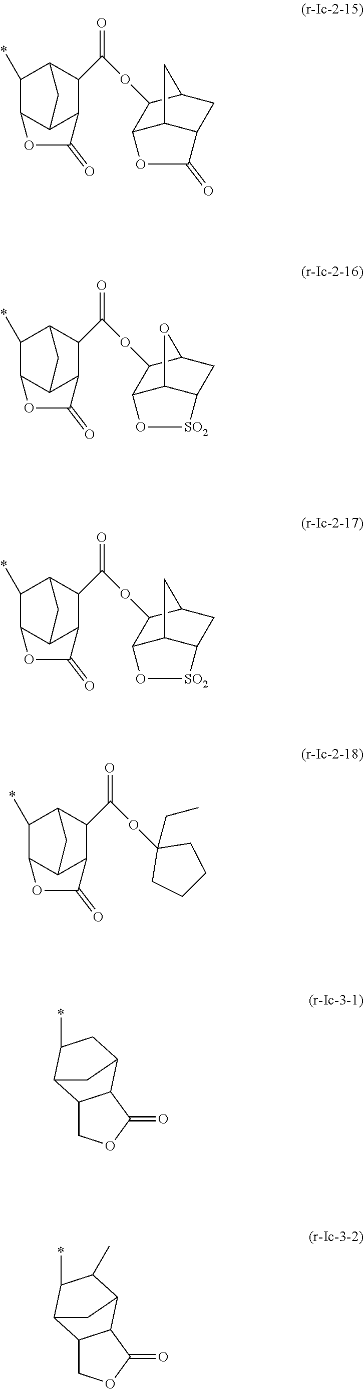

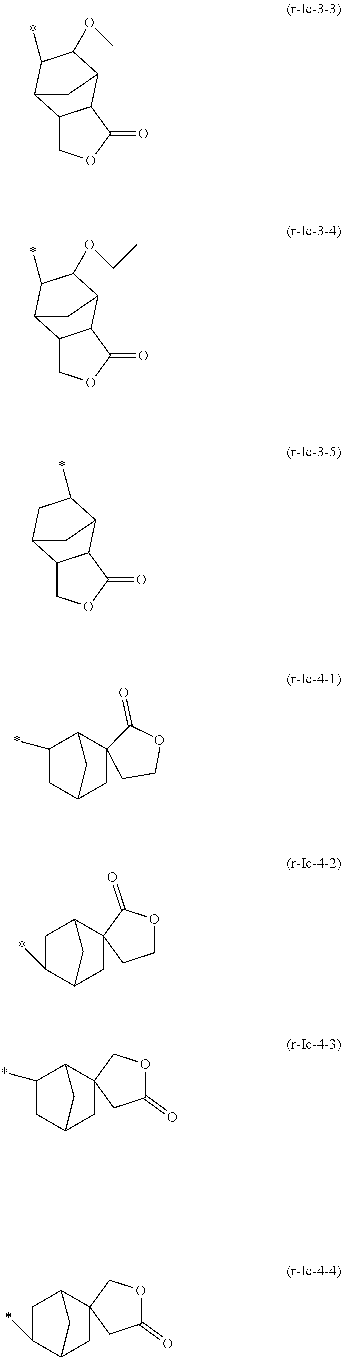

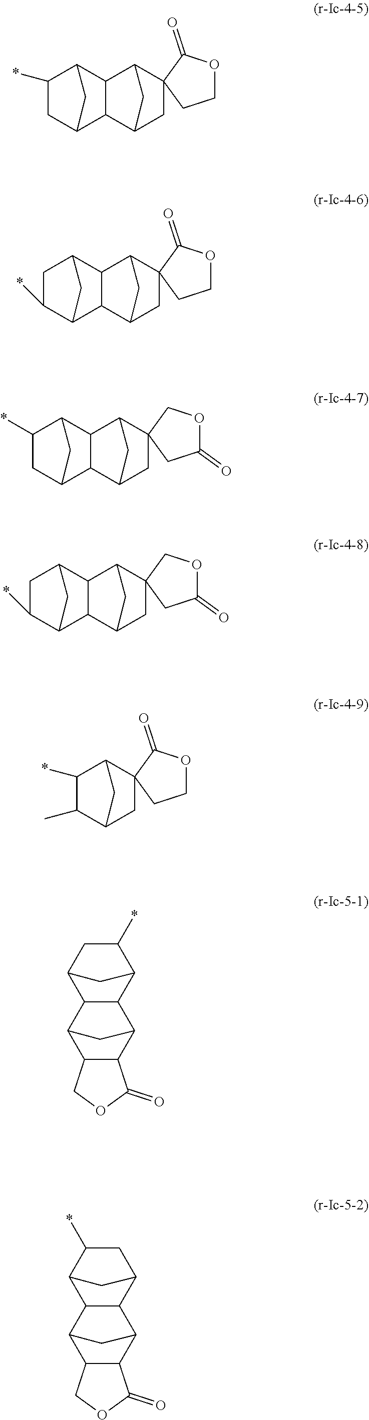

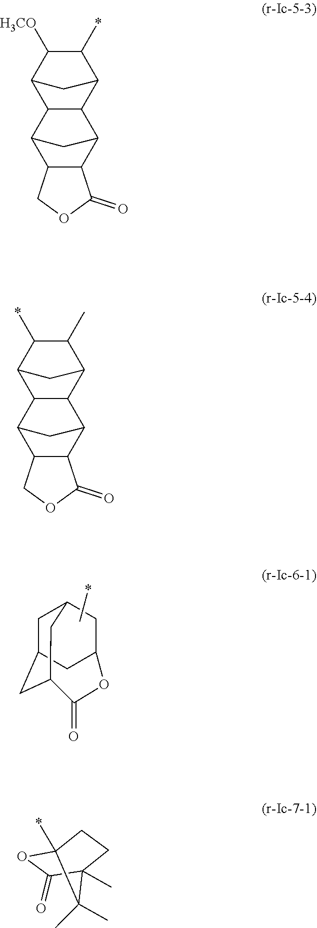

As the constitutional unit (a2), a constitutional unit derived from acrylic acid ester in which the hydrogen atom bonded to the carbon atom at the c-position may be substituted with a substituent is preferable.

It is preferable that such a constitutional unit (a2) be a constitutional unit represented by General Formula (a2-1).

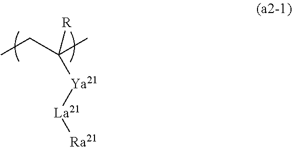

[In the formula, R represents a hydrogen atom, an alkyl group having 1 to 5 carbon atoms, or a halogenated alkyl group having 1 to 5 carbon atoms. Ya⁰ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a hetero atom.

It is preferable that Ya²¹ represent a single bond, an ester bond [—C(═O)—O—], an ether bond (—O—), a linear or branched alkylene group, or a combination thereof.

In Formula (a2-1), Ra²¹ represents a lactone-containing cyclic group.

Suitable examples of the lactone-containing cyclic group as Ra²¹ include groups represented by each of General Formulae (a2-r-1) to (a2-r-7).

Among these, groups represented by each of General Formulae (a2-r-1), (A2-r-2), and (a2-r-6) are preferable, and a group represented by General Formula (a2-r-2) is more preferable. Specifically, any one of groups represented by each of Chemical Formulae (r-1c-1-1) to (r-1c-1-7), (r-1c-2-1) to (r-1c-2-18), and (r-1c-6-1) is preferable, any one of groups represented by each of Chemical Formulae (r-1c-2-1) to (r-1c-2-18) is more preferable, and any one of groups represented by each of Chemical Formulae (r-1c-2-1) and (r-1c-2-12) is still more preferable.

The constitutional unit (a2) included in the component (A1) may be used alone or two or more kinds thereof may be used.

In a case where the component (A1) has the constitutional unit (a2), the proportion of the constitutional unit (a2) in the component (A1) is preferably in a range of 5% to 60% by mole, more preferably in a range of 10% to 60% by mole, still more preferably in a range of 20% to 60% by mole, and particularly preferably in a range of 30% to 60% by mole with respect to the total amount (100% by mole) of all constitutional units constituting the component (A1).

In a case where the proportion of the constitutional unit (a2) is set to be greater than or equal to the lower limits of the above-described preferable ranges. the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

In regard to constitutional unit (a8):

The constitutional unit (a8) is a constitutional unit derived from a compound represented by General Formula (a8-1).

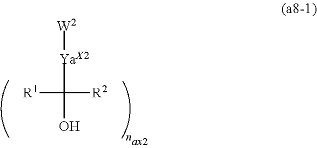

[In the formula, W² represents a polymerizable group-containing group. Ya^{x2} represents a single bond or an (nₐₓ₂+1)-valent linking group. Ya^{x2} and W² may form a fused ring. R¹ represents a fluorinated alkyl group having 1 to 12 carbon atoms. R² represents an organic group having 1 to 12 carbon atoms which may have a fluorine atom or a hydrogen atom. R² and Ya^{x2} may be bonded to each other to form a ring structure. nₐₓ₂ represents an integer of 1 to 3.]

The term “polymerizable group” in the polymerizable group-containing group as W² denotes a group that enables a compound containing a polymerizable group to be polymerized by radical polymerization or the like, which is, for example, a group having a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group having a hetero atom.

Suitable examples of the polymerizable group-containing group include a group represented by Chemical Formula: C(R^{X11})(R^{X12})═C(R^{X13})—Ya^{x0}.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the fused ring formed by Ya^{x2} and W² include a fused ring formed by a polymerizable group of the W² moiety and by Ya^{x2} and a fused ring formed by a group other than the polymerizable group of the W² moiety and by Ya^{x2}.

The fused ring formed by Ya^{x2} and W² may have a substituent.

Specific examples of the constitutional unit (a8) are shown below.

In the following formulae, R^{α} represents a hydrogen atom, a methyl group, or a trifluoromethyl group.

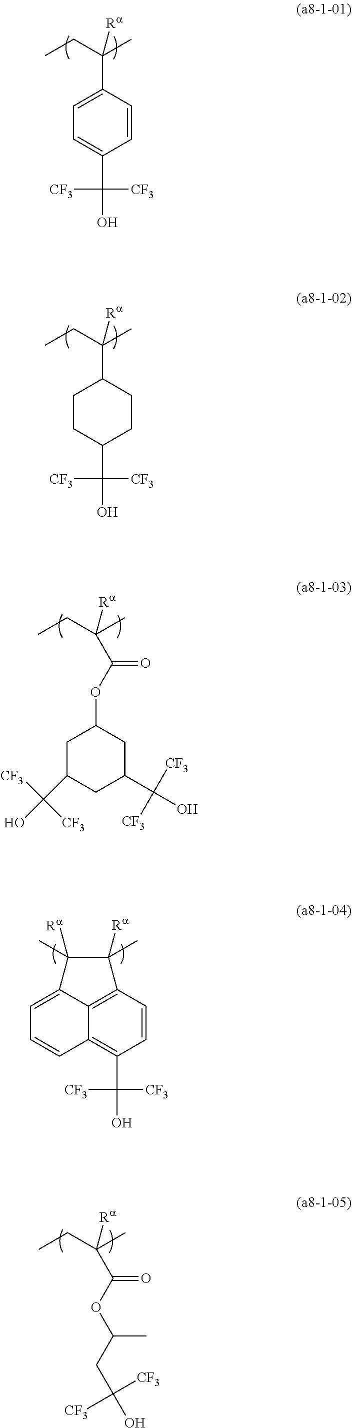

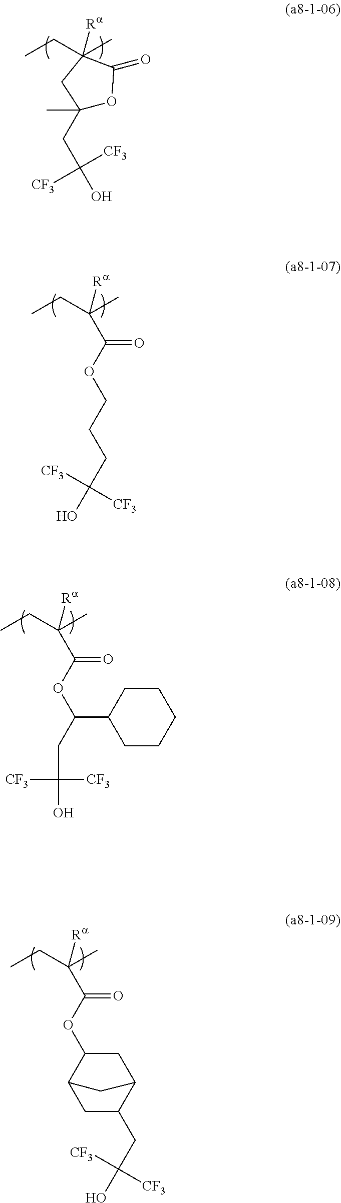

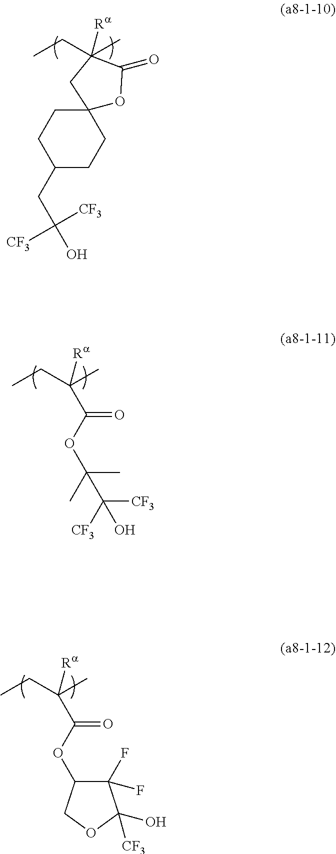

Among the examples, the constitutional unit (a8) is preferably at least one selected from the group consisting of constitutional units represented by each of Chemical Formulae (a8-1-01l) to (a8-1-04), (a8-1-06), (a8-1-08), (a8-1-09), and (a8-1-10) and more preferably at least one selected from the group consisting of constitutional units represented by each of Chemical Formulae (a8-1-01) to (a8-1-04) and (a8-1-09).

The constitutional unit (a8) contained in the component (A1) may be used alone or two or more kinds thereof may be used.

In a case where the component (A l) has the constitutional unit (a8). the proportion of the constitutional unit (a8) is preferably in a range of I % to 50% by mole, more preferably in a range of 5% to 45% by mole, and still more preferably in a range of 5% to 40% by mole, with respect to the total amount (100% by mole) of all constitutional units constituting the component (A l).

In a case where the proportion of the constitutional unit (a8) is greater than or equal to the lower limits of the above-described preferable ranges, the compatibility with the developing solution and the rinse solution can be enhanced. On the other hand, in a case where the proportion is less than or equal to the upper limits of the above-described preferable ranges, balance with other constitutional units can be obtained, and various lithography characteristics are improved.

In regard to constitutional unit (a01):

The constitutional unit (a01) is a constitutional unit derived from a compound represented by General Formula (a0-1).

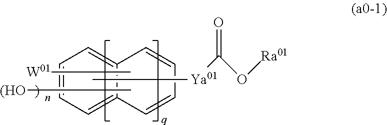

[In the formula, W⁰¹ represents a polymerizable group-containing group. Ya⁰¹ represents a single bond or a divalent linking group. Ra⁰¹ represents an acid dissociable group. q represents an integer of 0 to 3. n represents an integer of 1 or more. Here, n≤q×2+4 is satisfied.]

In Formula (a0-1), W⁰¹ represents a polymerizable group-containing group. The term “polymerizable group” in W⁰¹ denotes a group that enables a compound containing a polymerizable group to be polymerized by radical polymerization or the like, which is a group having a multiple bond between carbon atoms, such as an ethylenic double bond.

In the constitutional unit (a01), the multiple bonds in the polymerizable group are cleaved to form a main chain.

Examples of the polymerizable group as W⁰¹ include a vinyl group, an allyl group, an acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, a vinylnaphthyl group, a fluorine-containing styryl group, a fluorine-containing vinylnaphthyl group, a norbornyl group, a fluorine-containing norbornyl group, and a silyl group.

The term “polymerizable group-containing group” in W⁰¹ may denote a group formed of only a polymerizable group or a group formed of a polymerizable group and a group other than the polymerizable group. Examples of the group other than the polymerizable group include a divalent hydrocarbon group which may have a substituent and a divalent linking group having a hetero atom.

Divalent Hydrocarbon Group which May have Substituent:

In a case where the group other than the polymerizable group represents a divalent hydrocarbon group which may have a substituent, the hydrocarbon group may be an aliphatic hydrocarbon group or an aromatic hydrocarbon group.

Aliphatic Hydrocarbon Group as Group Other than Polymerizable Group

The aliphatic hydrocarbon group is a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The above linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a hetero atom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

Aromatic Hydrocarbon Group as Group Other than Polymerizable Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms. Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The above-described alkylene group bonded to the aryl group or heteroaryl group has preferably 1 to 4 carbon atoms, more preferably 1 or 2 carbon atoms, and particularly preferably 1 carbon atom.

The hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and most preferably a methyl group, an ethyl group, a propyl group, an n-butyl group, or a tert-butyl group.

Examples of the alkoxy group, the halogen atom, and the halogenated alkyl group as the substituents include those exemplified as the substituent that substitutes a hydrogen atom in the cyclic aliphatic hydrocarbon group.
Divalent linking group having hetero atom: In a case where the group other than the polymerizable group is a divalent linking group having a hetero atom, preferred examples of the linking group include —O—, —C(═O)—O—, —C(═O)—, —O—C(═O)—O——C(═O)—NH—, —NH—, —NH—C(═NH)— (H may be substituted with a substituent such as an alkyl group, an acyl group, or the like), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²— or —Y²¹—S(═O)₂—O—Y²²— [in the formulae, Y²¹ and Y²² each independently represent a divalent hydrocarbon group which may have a substituent, O represents an oxygen atom, and m″ represents an integer of 0 to 3].

In a case where the divalent linking group having a hetero atom is —C(═O)—NH—, —C(═O)—NH—C(═O)—, —NH—, or —NH—C(═NH)—, H may be substituted with a substituent such as an alkyl group or an acyl group. The substituent (an alkyl group, an acyl group, or the like) has preferably 1 to 10 carbon atoms, more preferably 1 to 8 carbon atoms, and particularly preferably 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹O—C(═O)—Y²²—, or —Y²¹—S(═O)₂—O—Y²²—, Y²¹ and Y²² each independently represent a divalent hydrocarbon group which may have a substituent. Examples of the divalent hydrocarbon group include the same groups as those for the divalent hydrocarbon group which may have a substituent, described in the section of the divalent linking group above.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

It is suitable for W¹ to represent, for example, a group represented by Chemical Formula: C(R^{X11})(R^{X12})═C(R^{X13})—Ya^{x0}—.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

The alkyl group having 1 to 5 carbon atoms as R^{X11}, R^{X12}, and R^{X13} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

Among these, R^{X11} and R^{X12} each represent preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom.

In addition, R^{X13} represents preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom.

The divalent linking group as Ya^{x0} is not particularly limited, and suitable examples thereof include a divalent hydrocarbon group which may have a substituent and a divalent linking group having a hetero atom, and these each have the same definition as described above.

Among the examples, it is preferable that Ya^{x0} represent an ester bond [—C(═O)—O— or —O—C(═O)—], an ether bond (—O—), a linear or branched alkylene group, an aromatic hydrocarbon group, or a combination thereof, or a single bond. Among these, Ya^{x0} represents more preferably a combination of an ester bond [—C(═O)—O— or —O—C(═O)—] and a linear alkylene group, or a single bond and still more preferably a single bond.

In Formula (a0-1), Ya⁰¹ represents a single bond or a divalent linking group. The divalent linking group as Ya⁰¹ is not particularly limited, and suitable examples thereof include a divalent hydrocarbon group which may have a substituent and a divalent linking group having a hetero atom, and these each have the same definition as described above.

Among the examples, in Formula (a0-1), it is preferable that Ya⁰¹ represent an ester bond [—C(═O)—O— or —O—C(═O)—], an ether bond (—O—), a linear or branched alkylene group, an aromatic hydrocarbon group, or a combination thereof, or a single bond. Among these, Ya⁰¹ represents more preferably a combination of an ester bond [—C(═O)—O— or —O—C(═O)—] and a linear alkylene group, or a single bond and still more preferably a single bond.

In Formula (a0-1), Ra⁰¹ represents an acid dissociable group.

Specific examples of the acid dissociable group include “acetal type acid dissociable group”, “tertiary alkyl ester type acid dissociable group”, and “secondary alkyl ester type acid dissociable group” described above.

Specific examples of the constitutional unit (a01) are shown below. In each of the formulae shown below, Rα represents a hydrogen atom, a methyl group, or a trifluoromethyl group.

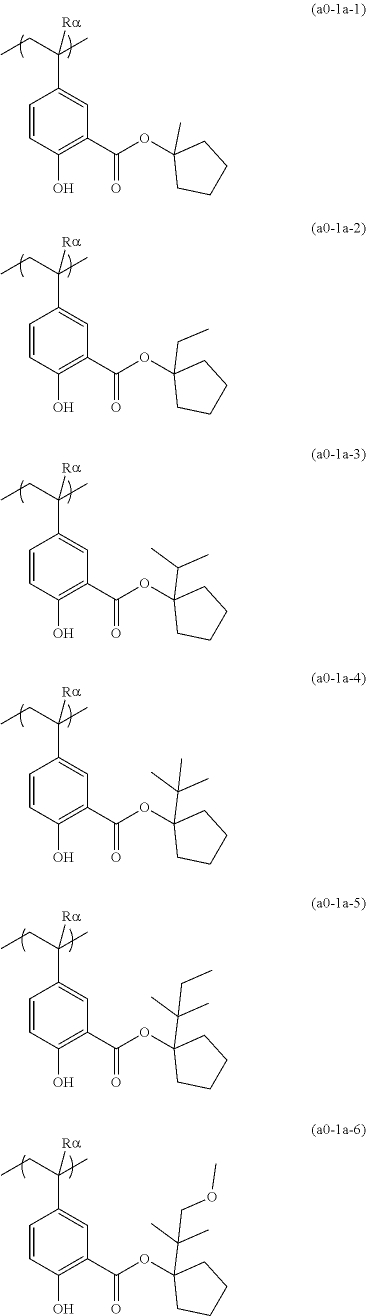

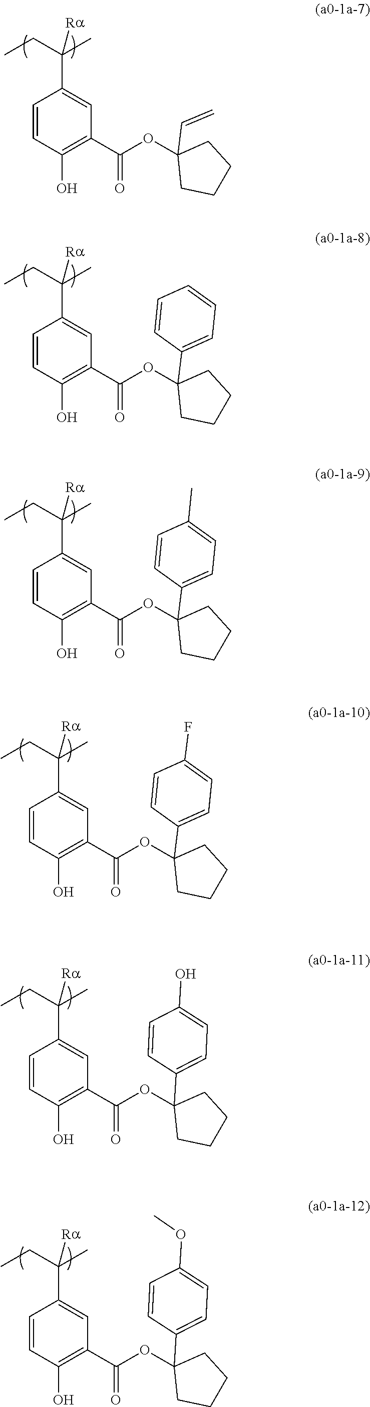

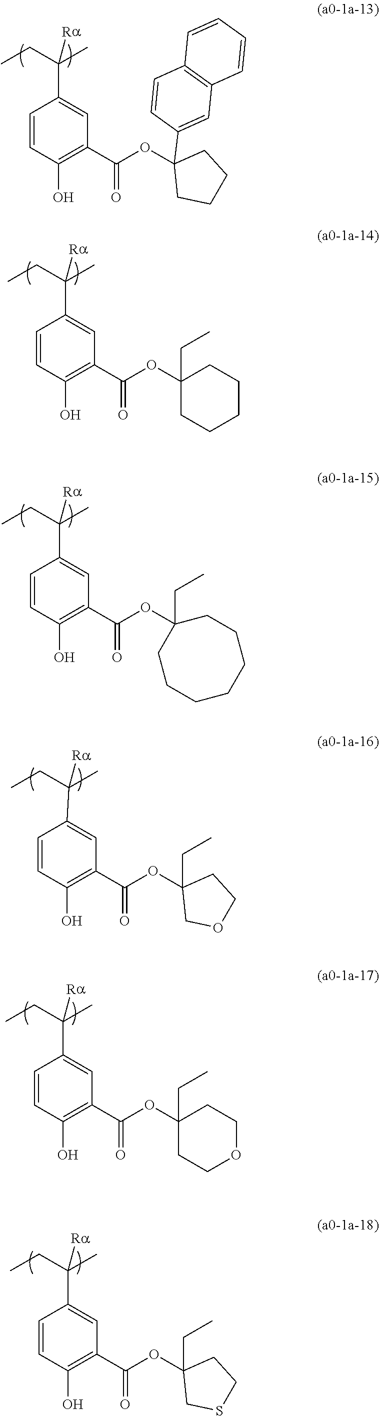

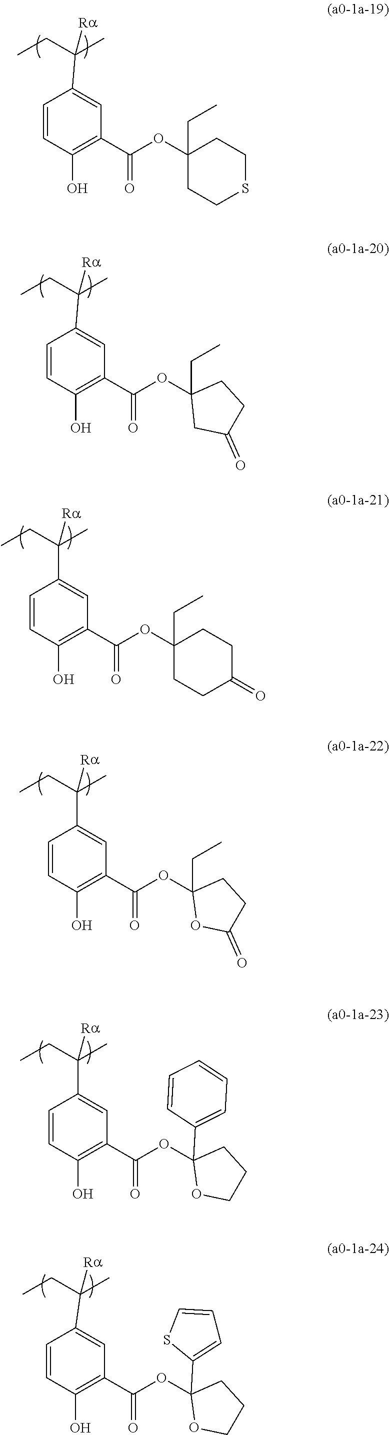

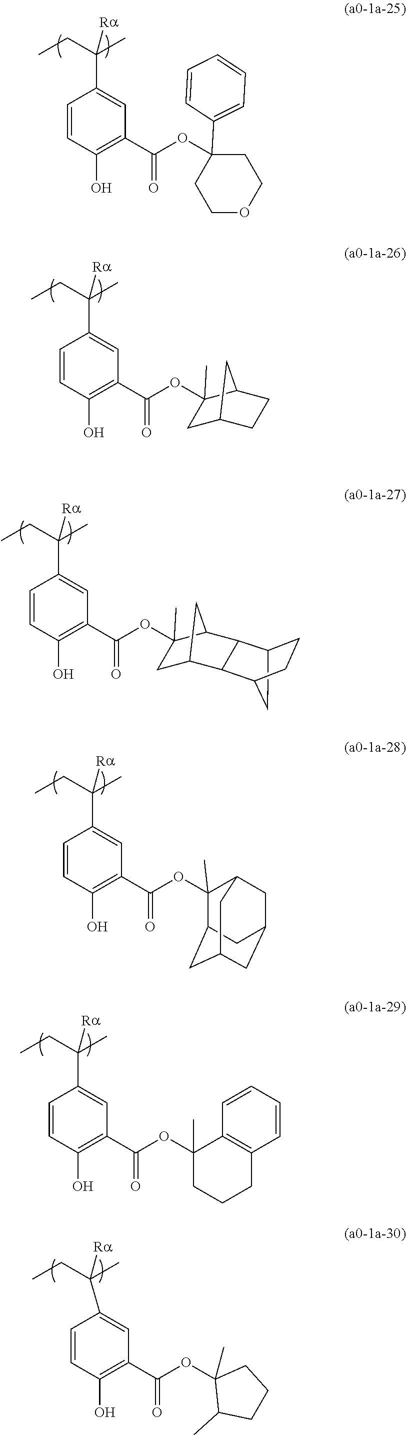

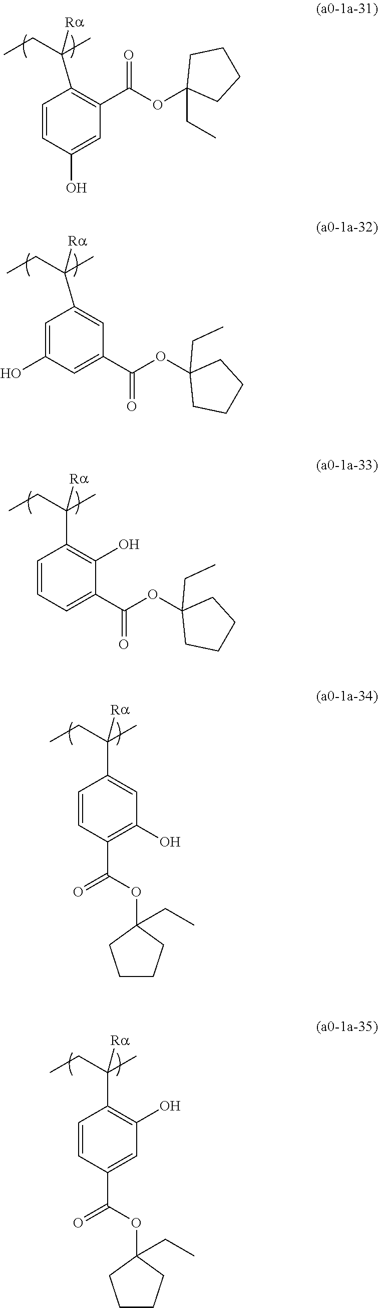

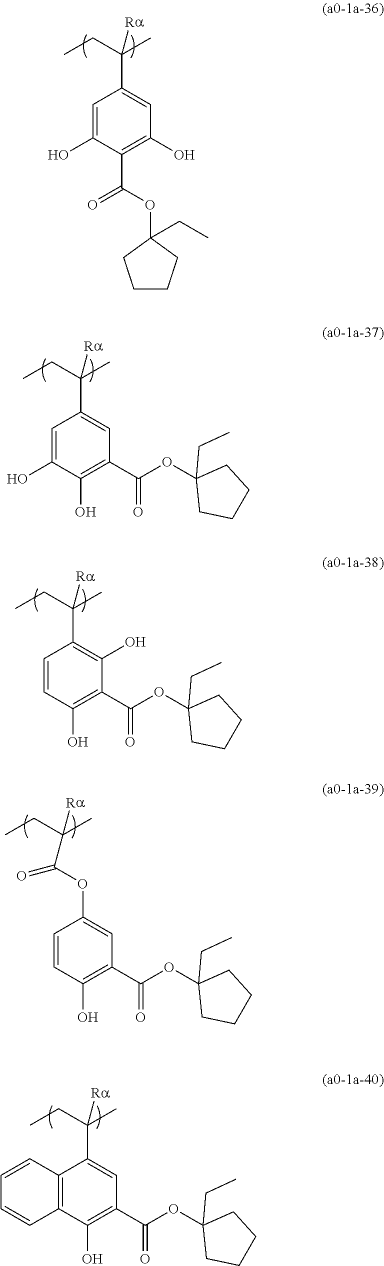

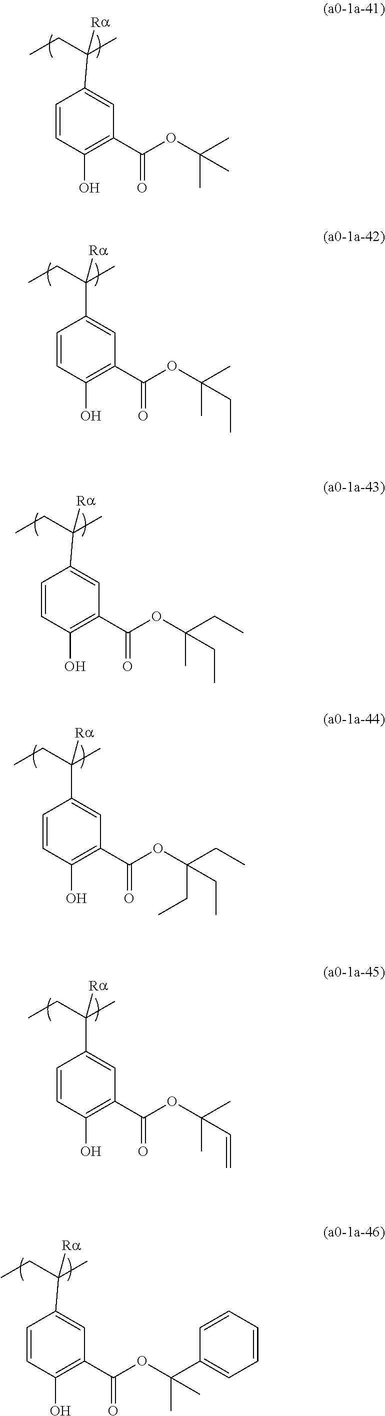

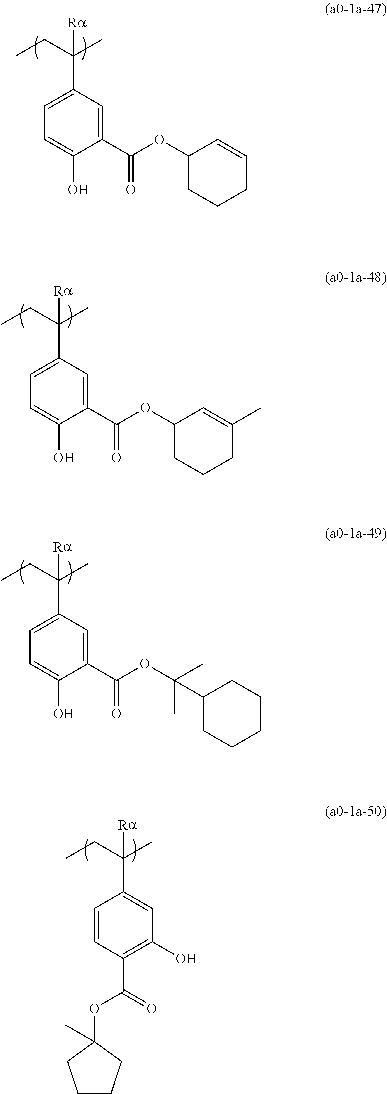

The constitutional unit (a01) in the component (A1) may be used alone or two or more kinds thereof may be used.

The proportion of the constitutional unit (a01) in the component (A1) is preferably in a range of 5% to 95% by mole, more preferably in a range of 10% to 90% by mole, still more preferably in a range of 30% to 70% by mole, and particularly preferably in a range of 40% to 60% by mole with respect to the total amount (100% by mole) of all constitutional units constituting the component (A1).

In a case where the proportion of the constitutional unit (a0l) is set to be greater than or equal to the lower limits of the above-described preferable ranges, lithography characteristics such as the sensitivity, CDU, the resolution, and reduction of the roughness are improved. Further, in a case where the proportion of the constitutional unit (a1) is less than or equal to the upper limits of the above-described preferable ranges, the constitutional unit (a1) and other constitutional units can be balanced, and the lithography characteristics are improved.

The component (A1) contained in the resist composition may be used alone or in a combination of two or more kinds thereof.

In the resist composition according to the present embodiment, examples of the component (A1) include a polymer compound having a repeating structure of the constitutional unit (a1), and preferred examples thereof include a polymer compound having a repeating structure of the constitutional unit (a1) and the constitutional unit (a10) and a polymer compound having a repeating structure of the constitutional unit (a01) and the constitutional unit (a10).

Among these, suitable examples of the component (A1) include a polymer compound consisting of a repeating structure of the constitutional unit (a 1) and the constitutional unit (a10) and a polymer compound consisting of a repeating structure of the constitutional unit (a01) and the constitutional unit (a10).

In the polymer compound having a repeating structure of the constitutional unit (a1) and the constitutional unit (a10), the proportion of the constitutional unit (a1) is preferably in a range of 10% to 90% by mole, more preferably in a range of 20% to 80% by mole, still more preferably in a range of 30% to 70% by mole, and particularly preferably in a range of 40% to 60% by mole with respect to the total amount (100% by mole) of all constitutional units constituting the polymer compound.

In addition, the proportion of the constitutional unit (a10) in each of the polymer compounds described above is preferably in a range of 10% to 90% by mole, more preferably in a range of 20% to 80% by mole, still more preferably in a range of 30% to 70% by mole, and particularly preferably in a range of 40% to 60% by mole, with respect to the total amount (100% by mole) of all constitutional units constituting the polymer compound.

The molar ratio of the constitutional unit (a1) to the constitutional unit (a10) (constitutional unit (a1):constitutional unit (a10)) in the polymer compound is preferably 2:8 to 8:2, more preferably 3:7 to 7:3, and still more preferably 4:6 to 6:4.

In the polymer compound having a repeating structure of the constitutional unit (a01) and the constitutional unit (a10), the proportion of the constitutional unit (a01) is preferably in a range of 10% to 90% by mole, more preferably in a range of 20% to 80% by mole, still more preferably in a range of 30% to 70% by mole, and particularly preferably in a range of 40% to 60% by mole with respect to the total amount (100% by mole) of all constitutional units constituting the polymer compound.

In addition, the proportion of the constitutional unit (a10) in each of the polymer compounds described above is preferably in a range of 10% to 90% by mole, more preferably in a range of 20% to 80% by mole, still more preferably in a range of 30% to 70% by mole, and particularly preferably in a range of 40% to 60% by mole, with respect to the total amount (100% by mole) of all constitutional units constituting the polymer compound.

The molar ratio of the constitutional unit (a01) to the constitutional unit (a10) (constitutional unit (a01):constitutional unit (a10)) in the polymer compound is preferably in a range of 2:8 to 8:2, more preferably in a range of 3:7 to 7:3, and still more preferably in a range of 4:6 to 6:4.

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a1) is derived and, as necessary, a monomer from which a constitutional unit other than the constitutional unit (a1) (for example, the constitutional unit (a10)) is derived, adding thereto a radical polymerization initiator as described above to carry out polymerization, and then carrying out a deprotection reaction.

Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, the resist composition exhibits a satisfactory solubility in a resist solvent for a resist enough to be used as a resist. On the other hand, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which are known as base material components for a chemically amplified resist composition and used.

As the component (A2), a polymer compound or a low-molecular-weight compound may be used alone or in a combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, high resolution, and improved roughness is likely to be formed.

In the resist composition of the present embodiment, the content of the component (A) may be adjusted according to the thickness of the resist film intended to be formed.

### <Acid Generator Component (B)>

The component (B) in the resist composition according to the present embodiment contains a compound (B0) represented by General Formula (b0) (hereinafter, also referred to as “component (B0)”).

### <<Compound (B0)>>

The component (B0) is a compound represented by General Formula (b0).

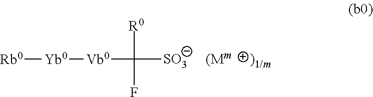

[In the formula, Rb⁰ represents a fused cyclic group in which an aromatic ring and an alicyclic ring are fused. The alicyclic ring in the fused cyclic group has substituents, and at least one of the substituents contains a hydrocarbon group having a bromine atom or a hydrocarbon group having an iodine atom. Yb⁰ represents a divalent linking group or a single bond. Here, Yb⁰ is bonded to the alicyclic ring in the fused cyclic group. Vb⁰ represents a single bond, an alkylene group, or a fluorinated alkylene group. R⁰ represents a fluorinated alkyl group having 1 to 5 carbon atoms or a fluorine atom. M^{m+} represents an m-valent organic cation. m represents an integer of 1 or greater.]

### {Anion Moiety of Component (B0)}

In General Formula (b0), Rb⁰ represents a fused cyclic group in which an aromatic ring and an alicyclic ring are fused.

The aromatic ring is not particularly limited as long as it is a cyclic conjugated system having 4n+2π electrons, and may be monocyclic or polycyclic. The aromatic ring has preferably 5 to 30 carbon atoms, more preferably 5 to 20 carbon atoms, still more preferably 6 to 15 carbon atoms, and particularly preferably 6 to 14 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

The alicyclic ring may be monocyclic or polycyclic. The alicyclic ring has preferably 4 to 30 carbon atoms, more preferably 4 to 20 carbon atoms, still more preferably 4 to 15 carbon atoms, and particularly preferably 4 to 10 carbon atoms.

Specific examples of the alicyclic ring include a monocyclic aliphatic ring such as cyclobutene, cyclopentane, cyclohexane, or cyclooctane, a polycyclic aliphatic ring such as adamantane, norbornane, isobornane, tricyclodecane, or tetracyclododecane, and an aliphatic heterocyclic ring in which some of the carbon atoms constituting the monocyclic or polycyclic alicyclic ring have been substituted with hetero atoms. Examples of the hetero atom in the aliphatic heterocyclic ring include an oxygen atom, a sulfur atom, and a nitrogen atom. Specific examples of the aliphatic heterocyclic ring include a tetrahydropyran ring, a thiane ring, and a piperidine ring.

The fused cyclic group as Rb⁰ may be formed such that one aromatic ring is fused with one alicyclic ring, two or more aromatic rings may be fused with one alicyclic ring, two or more alicyclic rings may be fused with one aromatic ring, or an alicyclic ring and an aromatic ring may be repeatedly fused. Further, in a case where a plurality of alicyclic rings and a plurality of aromatic rings are fused, each of these rings may be the same as or different from each other.

Among the examples, the fused cyclic group as Rb⁰ is preferably a fused cyclic group in which one aromatic ring is fused with one alicyclic ring or a fused cyclic group in which two or more aromatic rings are fused with one alicyclic ring, more preferably a fused cyclic group in which one aromatic hydrocarbon ring is fused with one monocyclic aliphatic ring or a fused cyclic group in which two or more aromatic hydrocarbon rings are fused with one monocyclic aliphatic ring, and still more preferably a fused cyclic group in which two aromatic hydrocarbon rings are fused with one monocyclic aliphatic ring.

Specific examples of the fused cyclic group as Rb⁰ include fluorene and a group in which one or more aromatic rings are fused with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane.

More specifically, the fused cyclic group as Rb⁰ is preferably a fused cyclic group in which two or three aromatic rings are fused with a bicycloalkane and more preferably a fused cyclic group in which two or three aromatic rings are fused with bicyclo[2.2.2]octane.

Specific examples of the fused cyclic group as Rb⁰ include groups represented by Formulae (r-br-1) and (r-br-2). In the formulae, * represents a bonding site that is bonded to Yb⁰ in Formula (b0).

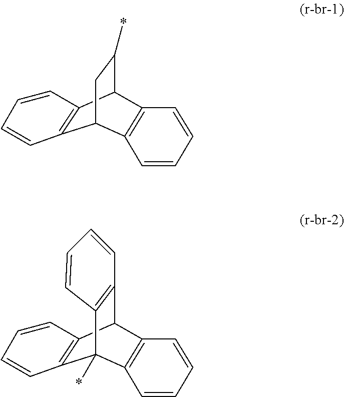 [SMILES: *C1CC2c3ccccc3C1c1ccccc12 ; *C12c3ccccc3C(c3ccccc31)c1ccccc12]

Among the examples, in General Formula (b0), the fused cyclic group as Rb⁰ is preferably a fused cyclic group in which two or three aromatic rings are fused with a bicycloalkane, more preferably a fused cyclic group in which two or three aromatic rings are fused with bicyclo[2.2.2]octane, and still more preferably a group represented by Formulae (r-br-1) and (r-br-2).

In General Formula (b0), the alicyclic ring in the fused cyclic group as Rb⁰ has substituents, and at least one of the substituents contains a hydrocarbon group having a bromine atom or a hydrocarbon group having an iodine atom.

Examples of the hydrocarbon group having a bromine atom or the hydrocarbon group having an iodine atom include a linear or branched alkyl group and a cyclic hydrocarbon group.

It is preferable that the linear alkyl group have 1 to 5 carbon atoms. Specific examples thereof include a methyl group, an ethyl group, an n-propyl group, an n-butyl group, and an n-pentyl group.

The branched alkyl group has preferably 3 to 10 carbon atoms and more preferably 3 to 5 carbon atoms. Specific examples include an isopropyl group, an isobutyl group, a tert-butyl group, an isopentyl group, a neopentyl group, a 1,1-diethylpropyl group, and a 2,2-dimethylbutyl group.

The cyclic hydrocarbon group may be an aliphatic hydrocarbon group or an aromatic hydrocarbon group, and may be a polycyclic group or a monocyclic group.

Examples of the cyclic hydrocarbon group include a group in which one hydrogen atom has been removed from an aromatic ring or an alicyclic ring in the fused cyclic group as Rb⁰.

Among the examples, as the hydrocarbon group having a bromine atom or the hydrocarbon group having an iodine atom, a cyclic hydrocarbon group is preferable, and an aromatic hydrocarbon group is more preferable.

The hydrocarbon group may have one or more substituents other than the bromine atom and the iodine atom. Examples of the substituents include an alkyl group, a fluorine atom, a chlorine atom, an alkoxy group (such as a methoxy group, an ethoxy group, a propoxy group, or a butoxy group), a hydroxy group, a cyano group, an amino group, and a nitro group.

Further, the hydrocarbon group may be formed such that some of the carbon atoms (such as a methylene group) constituting the hydrocarbon group may be substituted with a hetero atom-containing group.

Examples of the hetero atoms here include an oxygen atom, a nitrogen atom, and a sulfur atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

The hydrocarbon group may have both a bromine atom and an iodine atom. That is, the alicyclic ring in the fused cyclic group as Rb⁰ may contain a hydrocarbon group having a bromine atom and an iodine atom.

The total number of bromine atoms and iodine atoms in the hydrocarbon group is preferably an integer of 1 to 3, more preferably 2 or 3, and still more preferably 3.

As the total number of bromine atoms and iodine atoms in the hydrocarbon group increases, the sensitivity in the resist pattern formation tends to be increased.

Specifically, a group represented by General Formula (X-1) is preferable as the substituent contained in the alicyclic ring of the fused cyclic group as Rb⁰.

*—X⁰¹—Rᵢ⁰¹ (X-1)

[In the formula, X⁰¹ represents a single bond or a divalent linking group. Rᵢ⁰¹ represents a hydrocarbon group having a bromine atom or a hydrocarbon group having an iodine atom. * in the formula represents a bonding site bonded to the alicyclic ring in the fused cyclic group as Rb⁰ in Formula (b0).]

In General Formula (X-1), X⁰¹ represents a divalent linking group. Suitable examples of the divalent linking group include a divalent linking group having an oxygen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination.

Examples of the alkylene group include a linear alkylene group and a branched alkylene group.

Examples of the linear alkylene group include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

Examples of the branched alkylene group include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—, alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—, alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—.

Further, X⁰¹ may represent —N(R^{a})—C(═O)—, —N(R^{a})—, —C(R^{a})(R^{a})—N(R^{a})—, —C(R^{a})(N(R^{a})(R^{a}))—, —C(═O)—N(R^{a})—, or a group obtained by combining any of these groups and an alkylene group. Further, R^{a}'s each independently represent a hydrogen atom or an alkyl group.

In General Formula (X-1), X⁰¹ represents preferably —O—, —OCO—, —COO—, or a group obtained by combining any of these groups and an alkylene group, more preferably —OCO—, —COO—, or a group obtained by combining —OCO— or —COO— and an alkylene group, and still more preferably —COO—.

Further, in the specific examples of X⁰¹, the notation of each linking group matches the structure in General Formula (X-1). That is, for example, in regard to —COO—, the carbon atom of the alicyclic ring in the fused cyclic group as Rb⁰ is bonded to the carbon atom in —COO—. Further, Rᵢ⁰¹ in General Formula (X-1) is bonded to the oxygen atom in —COO—.

In General Formula (X-1), Rᵢ⁰¹ represents a hydrocarbon group having a bromine atom or a hydrocarbon group having an iodine atom, and the hydrocarbon group has the same definition as the hydrocarbon group having a bromine atom or the hydrocarbon group having an iodine atom.

Among the examples, in General Formula (X-1), Rᵢ⁰¹ represents preferably an aromatic hydrocarbon group having a bromine atom or an aromatic hydrocarbon group having an iodine atom, more preferably a phenyl group having a bromine atom or a naphthyl group having a bromine atom or a phenyl group having an iodine atom or a naphthyl group having an iodine atom, and still more preferably a phenyl group having a bromine atom or a phenyl group having an iodine atom.

In General Formula (X-1), Rᵢ⁰¹ may represent a hydrocarbon group having both a bromine atom and an iodine atom.

The total number of bromine atoms and iodine atoms in the hydrocarbon group is preferably an integer of 1 to 3, more preferably 2 or 3, and still more preferably 3.

As the total number of bromine atoms and iodine atoms in the hydrocarbon group increases, the sensitivity in the resist pattern formation tends to be increased.

The hydrocarbon group (aromatic hydrocarbon group) may have a substituent other than the bromine atom and the iodine atom. In a case where the hydrocarbon group (aromatic hydrocarbon group) has a substituent other than the bromine atom and the iodine atom, an alkyl group having 1 to 5 carbon atoms, a fluorine atom, or a hydroxy group is preferable as the substituent.

In General Formula (b0), Yb⁰ represents a divalent linking group or a single bond. Here, Yb⁰ is bonded to the alicyclic ring in the fused cyclic group.

Suitable examples of the divalent linking group as Yb⁰ include a divalent linking group containing an oxygen atom.

In a case where Yb⁰ represents a divalent linking group containing an oxygen atom, Yb⁰ may contain an atom other than the oxygen atom. Examples of the atom other than the oxygen atom include a carbon atom, a hydrogen atom, a sulfur atom, and a nitrogen atom.

Examples of the divalent linking group having an oxygen atom include those for the divalent linking group having an oxygen atom as X⁰¹ described above.

Examples of divalent linking groups containing an oxygen atom include linking groups represented by each of General Formulae (y-a1-1) to (y-a1-8) shown below. V′¹⁰¹ in General Formulae (y-a1-1) to (y-a1-7) is bonded to the alicyclic ring in the fused cyclic group as Rb⁰ in General Formula (b0).

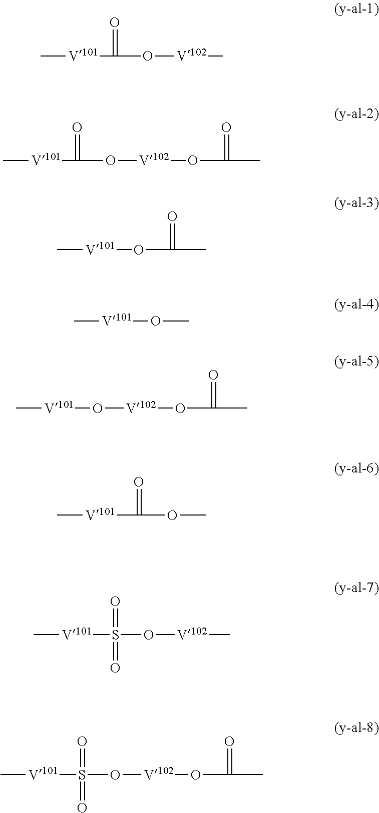

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V^{m+} and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—1.

Further, a part of the methylene group in the alkylene group as V′¹⁰¹ and V′¹⁰² may be substituted with a divalent aliphatic cyclic group having 5 to 10 carbon atoms. The aliphatic cyclic group is preferably a cyclohexylene group, a 1,5-adamantylene group, or a 2,6-adamantylene group.

Yb⁰ represents preferably a divalent linking group having an ester bond or a divalent linking group having an ether bond, more preferably a linking group represented by any of Formulae (y-a1-1) to (y-a1-6), and still more preferably a linking group represented by Formula (y-a1-1) or (y-a1-6).

In General Formula (b0), V^{b0} represents an alkylene group, a fluorinated alkylene group, or a single bond.

The alkylene group and the fluorinated alkylene group as V^{b0} each have preferably 1 to 4 carbon atoms and more preferably 1 to 3 carbon atoms. Examples of the fluorinated alkylene group as V^{b0} include a group in which some or all hydrogen atoms in the alkylene group have been substituted with fluorine atoms.

In General Formula (b0), V^{b0} represents preferably an alkylene group or a fluorinated alkylene group, more preferably an alkylene group having 1 to 4 carbon atoms or a fluorinated alkylene group having 1 to 4 carbon atoms, and still more preferably a linear alkylene group having 1 to 4 carbon atoms or a branched fluorinated alkylene group having 1 to 4 carbon atoms.

In General Formula (b0), R⁰ represents a fluorinated alkyl group having 1 to 5 carbon atoms or a fluorine atom. R⁰ represents preferably a fluorine atom or a perfluoroalkyl group having 1 to 5 carbon atoms and more preferably a fluorine atom.

In the present embodiment, from the viewpoint of improving the sensitivity and CDU, an anion represented by General Formula (b0-an0) is preferable as the anion moiety of the component (B0).

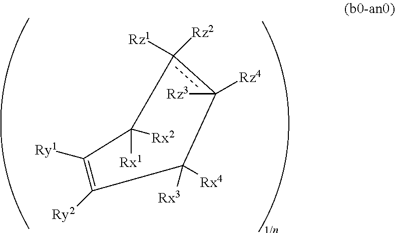

[In the formula. Rx¹ to Rx⁴ each independently represent a hydrocarbon group which may have a substituent or a hydrogen atom, or two or more thereof may be bonded to each other to form a ring structure. Ry¹ and Ry² each independently represent a hydrocarbon group which may have a substituent or a hydrogen atom, or may be bonded to each other to form a ring structure.

[symbol]

The doublet line of the dotted line and the straight line shown above represents a double bond or a single bond. Where valence permits, Rz¹ to Rz⁴ each independently represent a hydrocarbon group which may have a substituent or a hydrogen atom, or two or more thereof may be bonded to each other to form a ring structure. Here, at least one of two or more of Rx¹ to Rx⁴, Ry¹ and Ry², and two or more of Rz¹ to Rz⁴ are bonded to each other to form an aromatic ring. Further, at least one of Rx¹ to Rx⁴, Ry¹ and Ry², and Rz¹ to Rz⁴ has an anion group represented by General Formula (b0-r-an1), and the entire anion moiety is an n-valent anion. Further, at least one of Rx¹ to Rx⁴, Ry¹ and Ry², and Rz¹ to Rz⁴ contains a hydrocarbon group having a bromine atom or a hydrocarbon group having an iodine atom. n represents an integer of 1 or greater.]

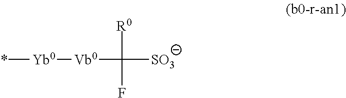

[In the formula, Yb⁰ represents a divalent linking group or a single bond. Vb⁰ represents a single bond, an alkylene group, or a fluorinated alkylene group. * represents a bonding site.]

In Formula (b0-an0), Rx¹ to Rx⁴ each independently represent a hydrocarbon group which may have a substituent or a hydrogen atom, or two or more thereof may be bonded to each other to form a ring structure.

Ry¹ and Ry² each independently represent a hydrocarbon group which may have a substituent or a hydrogen atom, or may be bonded to each other to form a ring structure.

Where valence permits, Rz¹ to Rz⁴ each independently represent a hydrocarbon group which may have a substituent or a hydrogen atom, or two or more thereof may be bonded to each other to form a ring structure.

The hydrocarbon groups as Rx¹ to Rx⁴, Ry¹, Ry², and Rz¹ to Rz⁴ may each be an aliphatic hydrocarbon group or an aromatic hydrocarbon group or may each be a cyclic hydrocarbon group or a chain-like hydrocarbon group.

Examples of the hydrocarbon group which may have a substituent as Rx¹ to Rx⁴, Ry¹, Ry², and Rz¹ to Rz⁴ include a cyclic group which may have a substituent, a chain-like alkyl group which may have a substituent, and a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated. Further, the cyclic hydrocarbon group as Rx¹ to Rx⁴, Ry¹, Ry², and Rz¹ to Rz⁴ may contain a hetero atom such as a heterocyclic ring.

The aromatic hydrocarbon group as Rx¹ to Rx⁴, Ry¹, Ry², and Rz¹ to Rz⁴ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group as Rx¹ to Rx⁴, Ry¹, Ry², and Rz¹ to Rz⁴ include benzene, fluorene, naphthalene, anthracene, phenanthrene, biphenyl, and an aromatic heterocyclic ring in which some of the carbon atoms constituting any one of these aromatic rings have been substituted with hetero atoms. Examples of the hetero atom in the aromatic heterocyclic rings include an oxygen atom, a sulfur atom, and a nitrogen atom. The aromatic ring contained in the aromatic hydrocarbon group as Rx¹ to Rx⁴, Ry¹, Ry², and Rz¹ to Rz⁴ is preferably an aromatic ring containing no hetero atoms and more preferably an aromatic ring such as benzene, fluorene, naphthalene, anthracene, phenanthrene, or biphenyl from the viewpoint of the compatibility with the component (A).

Specific examples of the aromatic hydrocarbon group as Rx¹ to Rx⁴, Ry¹, Ry², and Rz¹ to Rz⁴ include a group in which one hydrogen atom has been removed from the above-described aromatic ring (an aryl group such as a phenyl group or a naphthyl group) and a group in which one hydrogen atom in the aromatic ring has been substituted with an alkylene group (an arylalkyl group such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as Rx¹ to Rx⁴, Ry¹, Ry², and Rz¹ to Rz⁴ include an aliphatic hydrocarbon group having a ring in the structure thereof. Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

In addition, examples of the cyclic group as Rx¹ to Rx⁴, Ry¹, Ry², and Rz¹ to Rz⁴ include —COOR^{XYZ} and —OC(═O)R^{XYZ}, where Rx^{XYZ} represents a lactone-containing cyclic group or a —SO₂-containing cyclic group.

The term “—SO₂-containing cyclic group” denotes a cyclic group having a ring containing —SO₂— in the ring skeleton thereof. Specifically, the —SO₂-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof.

More specific examples of the —SO₂-containing cyclic group include groups represented by each of General Formulae (b5-r-1) to (b5-r-4).

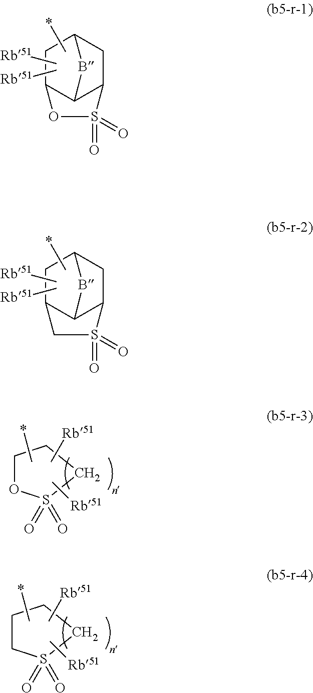

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, or a —SO₂-containing cyclic group, B″ represents an alkylene group having 1 to 5 carbon atoms which may have an oxygen atom or a sulfur atom, an oxygen atom, or a sulfur atom, and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group. Among these, it is preferable that Rb′¹'s each independently represent a hydrogen atom or a cyano group.

Specific examples of the groups represented by each of General Formulae (b5-r-1) to (b5-r-4) are shown below. In the formulae shown below, “Ac” represents an acetyl group.

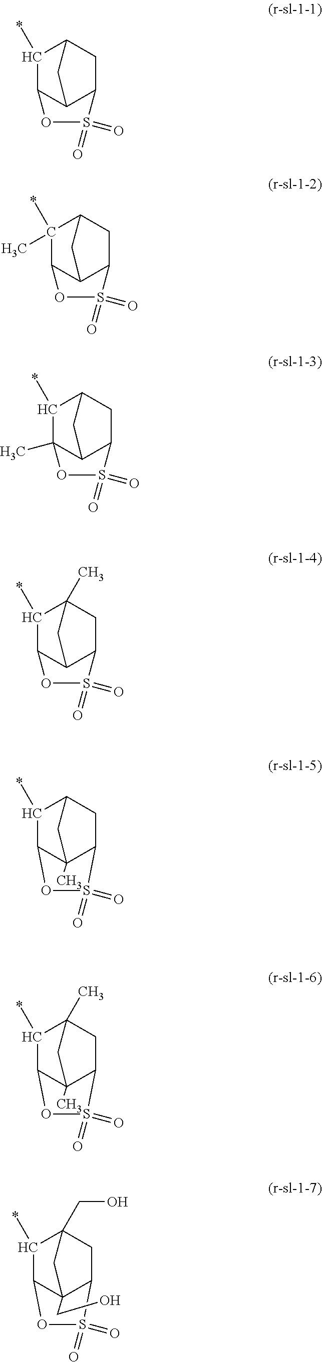

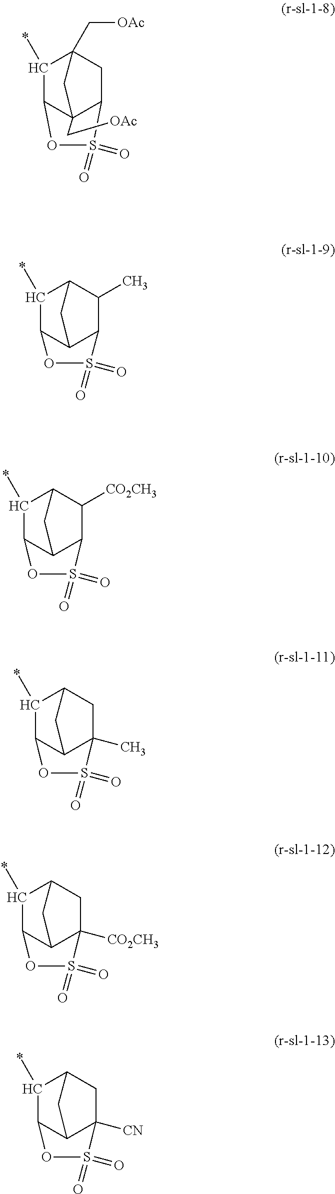

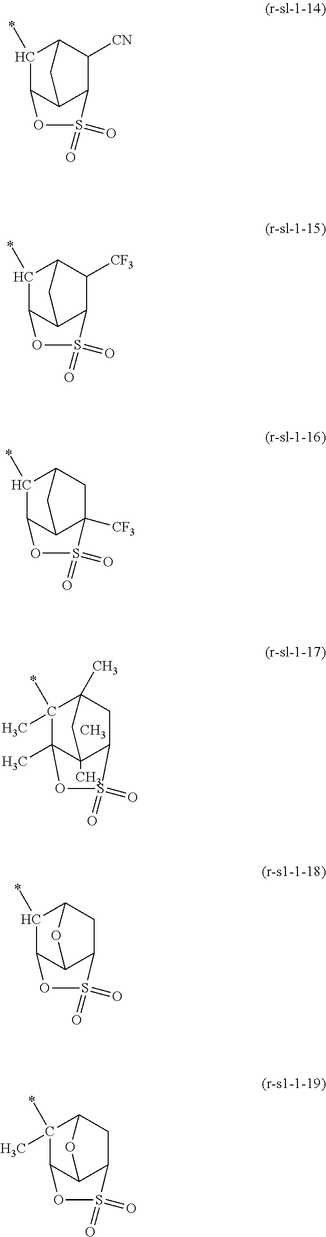

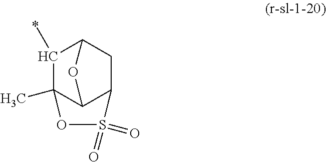

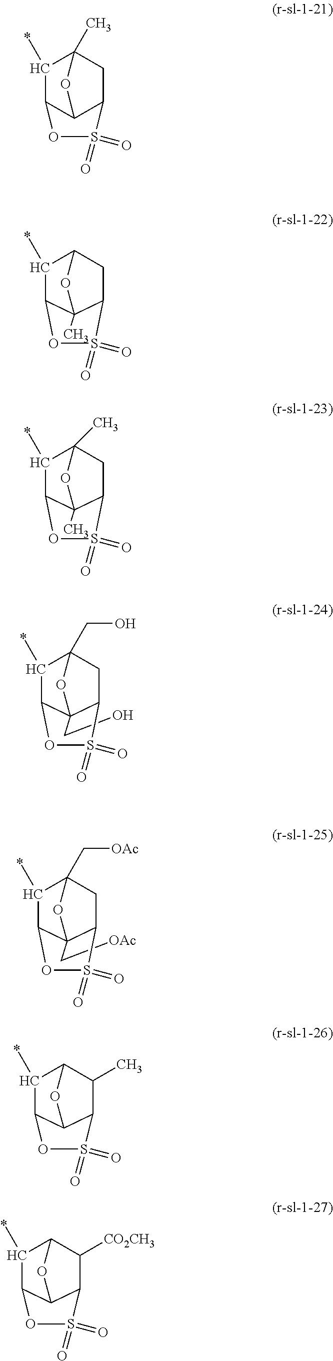

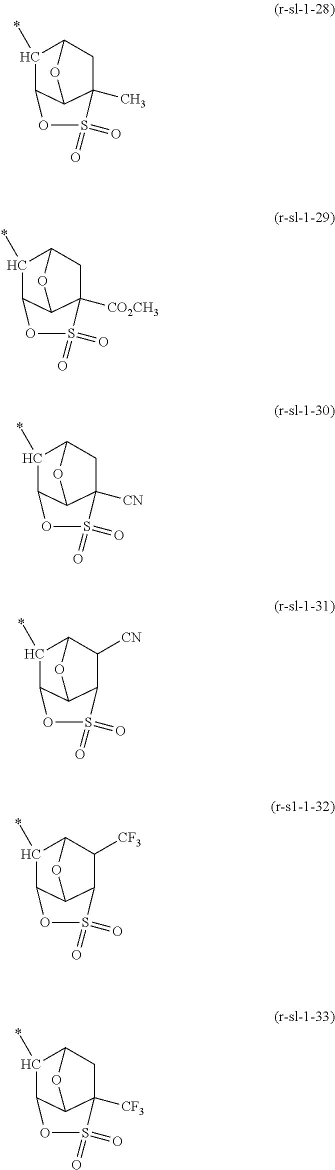

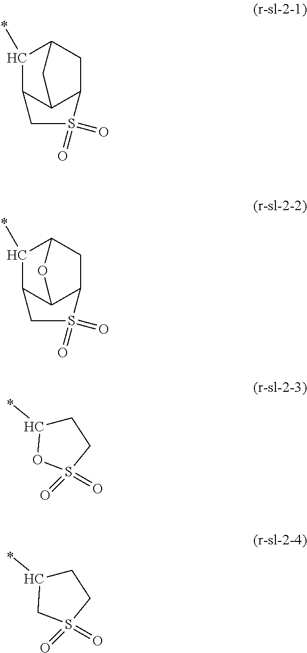

Examples of the substituent in the cyclic group as Rx¹ to Rx⁴, Ry¹, Ry², and Rz¹ to Rz⁴ include the same substituents as the substituents that the polycyclic aromatic cyclic group as Rb⁰ may have.

Among the examples, from the viewpoint of the compatibility with the component (A), an alkyl group, a halogen atom, and a halogenated alkyl group are preferable as the substituents in the cyclic group as Rx¹ to Rx⁴, Ry¹, Ry², and Rz¹ to Rz⁴.

Chain-Like Alkyl Group which May have Substituent:

The chain-like alkyl group as Rx¹ to Rx⁴, Ry¹, Ry², and Rz¹ to Rz⁴ may be any of linear or branched.

The linear alkyl group has preferably 1 to 20 carbon atoms, more preferably 1 to 15 carbon atoms, and most preferably 1 to 10 carbon atoms. Specific examples thereof include a methyl group, an ethyl group, a propyl group, a butyl group, a pentyl group, a hexyl group, a heptyl group, an octyl group, a nonyl group, a decanyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group, and a docosyl group.

The branched alkyl group has preferably 3 to 20 carbon atoms, more preferably 3 to 15 carbon atoms, and most preferably 3 to 10 carbon atoms. Specific examples thereof include a 1-methylethyl group, a 1,1-dimethylethyl group, a 1-methylpropyl group, a 2-methylpropyl group, a 1-methylbutyl group, a 2-methylbutyl group, a 3-methylbutyl group, a I-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as Rx¹ to Rx⁴, Ry¹, Ry², and Rz¹ to Rz⁴ may be linear or branched and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (an allyl group), and a butynyl group. Examples of the branched alkenyl group include a 1-propenyl group, a 2-propenyl group (an allyl group), a 1-methylpropenyl group, and a 2-methylpropenyl group.

Examples of the substituent in the chain-like alkyl group or alkenyl group as Rx¹ to Rx⁴, Ry¹, Ry², and Rz¹ to Rz⁴ include an alkoxy group, a halogen atom, a halogenated alkyl group, a hydroxyl group, a carbonyl group, a nitro group, an amino group, and the above-described cyclic groups as Rx¹ to Rx⁴, Ry¹, Ry², and Rz¹ to Rz⁴. Among these, from the viewpoint of the compatibility with the component (A), a halogen atom, a halogenated alkyl group, and the groups described as the cyclic group as Rx¹ to Rx⁴, Ry¹, Ry², and Rz¹ to Rz⁴ are preferable as the substituents in the chain-like alkyl group or alkenyl group as Rx¹ to Rx⁴, Ry¹, Ry², and Rz¹ to Rz⁴.

In Formula (b0-an0), Ry¹ and Ry² may be bonded to each other to form a ring structure.

Such a ring structure formed by Ry¹ and Ry² shares one side (the bond between carbon atoms to which each of Ry¹ and Ry² is bonded) of the 6-membered ring in Formula (b0-an0), and this ring structure may be an alicyclic hydrocarbon or an aromatic hydrocarbon. Further, this ring structure may be a polycyclic structure consisting of other ring structures.

The alicyclic hydrocarbon formed by Ry¹ and Ry² may be polycyclic or monocyclic. The monocyclic alicyclic hydrocarbon is preferably a monocycloalkane. The monocycloalkane has preferably 3 to 6 carbon atoms, and specific examples thereof include cyclopentane and cyclohexane. The polycyclic alicyclic hydrocarbon is preferably a polycycloalkane. It is preferable that the polycycloalkane have 7 to 30 carbon atoms.

Examples of the aromatic hydrocarbon ring formed by Ry¹ and Ry² include benzene, fluorene, naphthalene, anthracene, phenanthrene, biphenyl, and an aromatic heterocyclic ring in which some of the carbon atoms constituting any of these aromatic rings have been substituted with hetero atoms. The aromatic hydrocarbon ring formed by Ry¹ and Ry² is preferably an aromatic ring containing no hetero atoms and more preferably an aromatic ring such as benzene, fluorene, naphthalene, anthracene, phenanthrene, or biphenyl from the viewpoint of the compatibility with the component (A).

The ring structure (the alicyclic hydrocarbon or the aromatic hydrocarbon) formed by Ry¹ and Ry² may have a substituent. Examples of the substituent here include the same substituents as the substituents (such as an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among these, from the viewpoint of the compatibility with the component (A), an alkyl group, a halogen atom, or a halogenated alkyl group is preferable as the substituent in the ring structure formed by Ry¹ and Ry².

Among the examples, from the viewpoint of shortening the diffusion of the acid generated upon light exposure and the acid diffusion controllability, an aromatic hydrocarbon which may have a substituent is more preferable as the ring structure formed by Ry¹ and Ry².

In Formula (b0-an0), two or more of Rz¹ to Rz⁴ may be bonded to each other to form a ring structure. For example, Rz¹ may form a ring structure with any one of Rz² to Rz⁴. Specific examples thereof include a ring structure that shares one side (the bond between a carbon atom to which Rz¹ and Rz² are bonded and a carbon atom to which Rz³ and Rz⁴ are bonded) of the 6-membered ring in Formula (b0-an0), a ring structure formed by Rz¹ and Rz² being bonded to each other, and a ring structure formed by Rz³ and Rz⁴ being bonded to each other.

Such a ring structure formed by two or more of Rz¹ to Rz⁴ may be an alicyclic hydrocarbon or an aromatic hydrocarbon. Among these, an aromatic hydrocarbon is preferable. Further, this ring structure may be a polycyclic structure consisting of other ring structures.

The alicyclic hydrocarbon formed by two or more of Rz¹ to Rz⁴ may be polycyclic or monocyclic. The monocyclic alicyclic hydrocarbon is preferably a monocycloalkane. The monocycloalkane has preferably 3 to 6 carbon atoms, and specific examples thereof include cyclopentane and cyclohexane. The polycyclic alicyclic hydrocarbon is preferably a polycycloalkane. The polycycloalkane is preferably a polycycloalkane having 7 to 30 carbon atoms, and specifically, the polycycloalkane is more preferably a polycycloalkane having a crosslinked ring-based polycyclic skeleton, such as adamantane, norbornane, isobornane, tricyclodecane, or tetracyclododecane; or a polycycloalkane having a fused ring-based polycyclic skeleton, such as a cyclic group having a steroid skeleton.

It may be a heterocyclic structure obtained by substituting some carbon atoms with hetero atoms and particularly preferably a nitrogen-containing heterocyclic ring, and specific examples thereof include a cyclic imide.

Examples of the aromatic hydrocarbon ring formed by two or more of Rz¹ to Rz⁴ include benzene, fluorene, naphthalene, anthracene, phenanthrene, biphenyl, or an aromatic heterocyclic ring in which some of the carbon atoms constituting these aromatic rings have been substituted with hetero atoms. The aromatic hydrocarbon ring formed by two or more of Rz¹ to Rz⁴ is preferably an aromatic ring containing no hetero atoms and more preferably an aromatic ring such as benzene, fluorene, naphthalene, anthracene, phenanthrene, or biphenyl from the viewpoint of the compatibility with the component (A).

The ring structure (the alicyclic hydrocarbon or the aromatic hydrocarbon) formed by Rz¹ to Rz⁴ may have a substituent. Examples of the substituent here include the same substituents as the substituents (such as an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among these, from the viewpoint of the compatibility with the component (A), an alkyl group, a halogen atom, or a halogenated alkyl group is preferable as the substituent in the ring structure formed by Rz¹ to Rz⁴.

Among the examples, the ring structure formed by two or more of Rz¹ to Rz⁴ is preferably a ring structure that shares one side (the bond between a carbon atom to which Rz¹ and Rz² are bonded and a carbon atom to which Rz³ and Rz⁴ are bonded) of the 6-membered ring in Formula (b0-an0) and more preferably an aromatic ring structure from the viewpoint of the diffusion controllability of the acid generated upon light exposure.

In Formula (b0-an0), “where valence permits” has the following meaning. That is, in a case where the bond between the carbon atom to which Rz¹ and Rz² are bonded and the carbon atom to which Rz³ and Rz⁴ are bonded is a single bond, all of Rz¹, Rz², Rz³, and Rz⁴ are present. In a case where the bond between the carbon atom to which Rz¹ and Rz² are bonded and the carbon atom to which Rz³ and Rz⁴ are bonded is a double bond, only one of Rz¹ and Rz² is present, and only one of Rz³ and Rz⁴ is present. Further, for example, in a case where Rz¹ and Rz³ are bonded to form an aromatic ring structure, Rz² and Rz⁴ are not present.

In Formula (b0-an0), two or more of Rx¹ to Rx⁴ may be bonded to each other to form a ring structure. For example, Rx¹ may form a ring structure with any one of Rx² to Rx⁴.

Such a ring structure formed by two or more of Rx¹ to Rx⁴ may be an alicyclic hydrocarbon or an aromatic hydrocarbon. Further, this ring structure may be a polycyclic structure consisting of other ring structures.

The alicyclic hydrocarbon formed by two or more of Rx¹ to Rx⁴ may be polycyclic or monocyclic. The monocyclic alicyclic hydrocarbon is preferably a monocycloalkane. The monocycloalkane has preferably 3 to 6 carbon atoms, and specific examples thereof include cyclopentane and cyclohexane. The polycyclic alicyclic hydrocarbon is preferably a polycycloalkane. The polycycloalkane is preferably a polycycloalkane having 7 to 30 carbon atoms, and specifically, the polycycloalkane is more preferably a polycycloalkane having a crosslinked ring-based polycyclic skeleton, such as adamantane, norbornane, isobornane, tricyclodecane, or tetracyclododecane; or a polycycloalkane having a fused ring-based polycyclic skeleton, such as a cyclic group having a steroid skeleton.

Examples of the aromatic hydrocarbon ring formed by two of Rx¹ to Rx⁴ include benzene, fluorene, naphthalene, anthracene, phenanthrene, biphenyl, or an aromatic heterocyclic ring in which some of the carbon atoms constituting any of these aromatic rings have been substituted with hetero atoms. The aromatic hydrocarbon ring formed by two of Rx¹ to Rx⁴ is preferably an aromatic ring containing no hetero atoms and more preferably an aromatic ring such as benzene, fluorene, naphthalene, anthracene, phenanthrene, or biphenyl from the viewpoint of the compatibility with the component (A).

The ring structure (the alicyclic hydrocarbon or the aromatic hydrocarbon) formed by Rx¹ to Rx⁴ may have a substituent. Examples of the substituent here include the same substituents as the substituents (such as an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among these, from the viewpoint of the compatibility with the component (A), an alkyl group, a halogen atom, or a halogenated alkyl group is preferable as the substituent in the ring structure formed by Rx¹ to Rx⁴.

From the viewpoint of the acid diffusion controllability, an alicyclic hydrocarbon is preferable as the ring structure formed by two or more of Rx¹ to Rx⁴.

Further, among the examples, the ring structure formed by two or more of Rx¹ to Rx⁴ is preferably a ring structure in which at least one of Rx¹ and Rx² and at least one of Rx³ to Rx⁴ are bonded to each other to form a crosslinked ring structure and more preferably an alicyclic hydrocarbon from the viewpoint of the acid diffusion controllability.

In a case where at least one of Rx¹ and Rx² and at least one of Rx³ and Rx⁴ are bonded to each other to form a ring structure, the number of carbon atoms constituting the bicyclic structure (the ring structure which also contains carbon atoms each bonded to Ry¹, Ry², Rz¹ and Rz², and Rz⁴ and Rz⁴) is preferably in a range of 7 to 16.

In Formula (b0-an0), two or more of Rx¹ to Rx⁴, Ry¹ and Ry², or two or more of Rz¹ to Rz⁴ are bonded to each other to form an aromatic ring. The aromatic ring has the same definition as the aromatic ring described in the section of General Formula (b0).

In Formula (b0-an0), at least one of Rx¹ to Rx⁴, Ry¹, Ry², and Rz¹ to Rz⁴ has an anion group represented by General Formula (b0-r-an1), and the entire anion moiety is an n-valent anion. n represents an integer of 1 or greater. Rx¹ to Rx⁴, Ry¹, Ry², and Rz¹ to Rz⁴ may each be the above-described anion group. In a case where two or more of Rx¹ to Rx⁴ are bonded to each other to form a ring structure, a carbon atom that forms the ring structure or a hydrogen atom bonded to the carbon atom may be substituted with the above-described anion group. In a case where two or more of Ry¹ and Ry² are bonded to each other to form a ring structure, a carbon atom that forms the ring structure or a hydrogen atom bonded to the carbon atom may be substituted with the above-described anion group. In a case where two or more of Rz¹ to Rz⁴ are bonded to each other to form a ring structure, a carbon atom that forms the ring structure or a hydrogen atom bonded to the carbon atom may be substituted with the above-described anion group.

In Formula (b0-r-an1), the divalent linking group as Yb⁰ is the same as the divalent linking group as Yb⁰ in General Formula (b0).

In Formula (b0-r-an1), the alkylene group or the fluorinated alkylene group as Vb⁰ is the same as the alkylene group or the fluorinated alkylene group as Vb⁰ in General Formula (b0).

In a case where Yb⁰ represents a single bond, specific examples of the anion group represented by Formula (b0-r-an1) include fluorinated alkyl sulfonate anions such as a trifluoromethanesulfonate anion and a perfluorobutanesulfonate anion.

In a case where Yb⁰ represents a divalent linking group having an oxygen atom, examples thereof include an anion represented by any of Formulae (b0-r-an11) to (b0-r-an13).

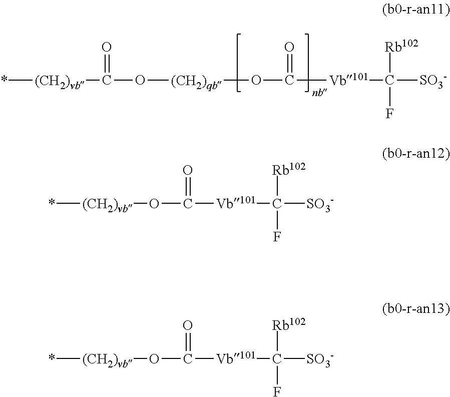

[In the formula, Vb″¹⁰¹ represents a single bond, an alkylene group having 1 to 4 carbon atoms, or a fluorinated alkylene group having 1 to 4 carbon atoms. Rb¹⁰² represents a fluorine atom or a fluorinated alkyl group having 1 to 5 carbon atoms. Each vb″ independently represents an integer of 0 to 3. Each qb″ independently represents an integer of 1 to 20. nb″ represents 0 or 1.]

In Formulae (b0-r-an11) to (b0-r-an13), Vb″¹⁰¹ represents a single bond, an alkylene group having 1 to 4 carbon atoms, or a fluorinated alkylene group having 1 to 4 carbon atoms. Vb″¹⁰¹ represents preferably a single bond, an alkylene group (methylene group) having 1 carbon atom, or a fluorinated alkylene group having 1 to 3 carbon atoms.

In Formulae (b0-r-an11) to (b0-r-an13), Rb¹⁰² represents a fluorine atom or a fluorinated alkyl group having 1 to 5 carbon atoms. Rb¹⁰² represents preferably a perfluoroalkyl group having 1 to 5 carbon atoms or a fluorine atom and more preferably a fluorine atom.

In Formulae (b0-r-an11) to (b0-r-an13), vb″ represents an integer in a range 0 to 3 and preferably represents 0 or 1.

qb″ represents an integer of 1 to 20, preferably represents an integer of I to 10, more preferably an integer of 1 to 5, still more preferably 1, 2, or 3, and particularly preferably 1 or 2.

nb″ represents 0 or 1 and preferably represents 0.

The number of anion groups in the component (B0) may be one or two or more.

In the component (B0), the entire anion moiety is an n-valent anion. n represents an integer of 1 or greater, preferably represents 1 or 2, and more preferably 1.

In Formula (b0-an0), at least one of Rx¹ to Rx⁴, Ry¹, Ry², and Rz¹ to Rz⁴ contains a hydrocarbon group having a bromine atom or a hydrocarbon group having an iodine atom. Preferable aspects of the hydrocarbon group having a bromine atom or the hydrocarbon group having an iodine atom are the same as the preferable aspects of the hydrocarbon group having a bromine atom or the hydrocarbon group having an iodine atom described in the section of General Formula (b0).

The anion moiety in the component (B0) is more preferably an anion represented by General Formula (b0-an1) from the viewpoint of acid diffusion controllability.

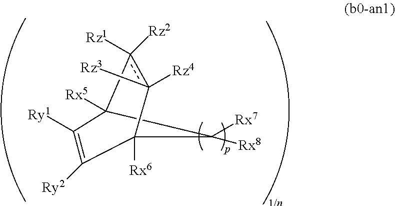

[In the formula, Rx⁵ and Rx⁶ each independently represent a hydrocarbon group which may have a substituent or a hydrogen atom. Rx⁷ and Rx⁸ each independently represent a hydrocarbon group which may have a substituent or a hydrogen atom or may be bonded to each other to form a ring structure. p represents 1 or 2, and in a case where p represents 2, a plurality of Rx⁷'s and Rx⁸'s may be different from each other. Ry¹ and Ry² each independently represent a hydrocarbon group which may have a substituent or a hydrogen atom, or may be bonded to each other to form a ring structure. [symbol] The doublet line of the dotted line and the straight line shown above represents a double bond or a single bond. Where valence permits, Rz¹ to Rz⁴ each independently represent a hydrocarbon group which may have a substituent or a hydrogen atom, or two or more thereof may be bonded to each other to form a ring structure. Here, Rx⁵ and Rx⁶, Rx⁷ and Rx⁸, Ry¹ and Ry², or two or more of Rz¹ to Rz⁴ are bonded to each other to form an aromatic ring. Further, at least one of Rx⁵ to Rx⁸, Ry¹, Ry², and Rz¹ to Rz⁴ contains an anion group represented by General Formula (b0-r-an1), and the entire anion moiety is an n-valent anion. Further, at least one of Rx⁵ to Rx⁸, Ry¹, Ry², and Rz¹ to Rz⁴ contains a hydrocarbon group having a bromine atom or a hydrocarbon group having an iodine atom. n represents an integer of 1 or greater.]

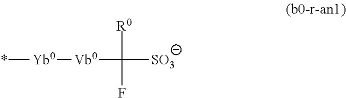

[In the formula, Yb⁰ represents a divalent linking group or a single bond. Vb⁰ represents a single bond, an alkylene group, or a fluorinated alkylene group. * represents a bonding site.]

In Formula (b0-an1), Rx⁵ and Rx⁶ each independently represent a hydrocarbon group which may have a substituent or a hydrogen atom. The hydrocarbon group which may have a substituent as Rx⁵ and Rx⁶ has the same definition as the hydrocarbon group which may have a substituent as Rx¹ to Rx⁴ in Formula (b0-an0).

In Formula (b0-an1), Rx⁷ and Rx⁸ each independently represent a hydrocarbon group which may have a substituent or a hydrogen atom or may be bonded to each other to form a ring structure. Rx⁷ and Rx⁸ each have the same definition as that for Rx¹ to Rx⁴ in Formula (b0-an0).

In Formula (b0-an1), p represents 1 or 2, and in a case where p represents 2, a plurality of Rx⁷'s and Rx⁸'s may be different from each other. In the case of p=1, the anion represented by General Formula (b0-an1) has a bicycloheptane ring structure, and in the case of p=2, the anion has a bicyclooctane ring structure.

In Formula (b0-an1), Ry¹ and Ry² each independently represent a hydrocarbon group which may have a substituent or a hydrogen atom or may be bonded to each other to form a ring structure. Ry¹ and Ry² have the same definitions as Ry¹ and Ry² in Formula (b0-an0), respectively.

Where valence permits, Rz¹ to Rz⁴ each independently represent a hydrocarbon group which may have a substituent or a hydrogen atom, or two or more thereof may be bonded to each other to form a ring structure. Rz¹ to Rz⁴ have the same definitions as Rz¹ to Rz⁴ in Formula (b0-an0), respectively.

In Formula (b0-an1), Rx⁵ and Rx¹, Rx⁷ and Rx¹, Ry¹ and Ry², or two or more of Rz¹ to Rz⁴ are bonded to each other to form an aromatic ring. The aromatic ring has the same definition as in the section of General Formula (b0).

In Formula (b0-an1), at least one of Rx⁵ to Rx⁸, Ry¹, Ry², and Rz¹ to Rz⁴ contains an anion group represented by Formula (b0-r-an1), and the entire anion moiety is an n-valent anion. n represents an integer of 1 or greater, preferably represents 1 or 2, and more preferably 1.

In Formula (b0-an1), at least one of Rx⁵ to Rx⁸, Ry¹ and Ry², and Rz¹ to Rz⁴ contains a hydrocarbon group having a bromine atom or a hydrocarbon group having an iodine atom. Preferable aspects of the hydrocarbon group having a bromine atom or the hydrocarbon group having an iodine atom are the same as the preferable aspects of the hydrocarbon group having a bromine atom or the hydrocarbon group having an iodine atom described in the section of General Formula (b0).

Among the examples, from the viewpoint of improving the sensitivity and CDU, an anion represented by Formula (b0-an1) in the case of p=2, that is, an anion represented by General Formula (b0-an2), is still more preferable as the anion moiety in the component (B0).

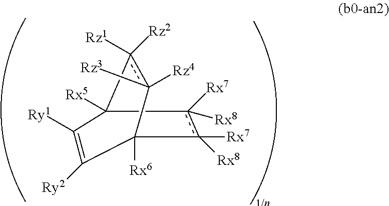

[In the formula, Rx⁵ and Rx⁶ each independently represent a hydrocarbon group which may have a substituent or a hydrogen atom. A plurality of Rx⁷'s and Rx⁸'s each independently represent a hydrocarbon group which may have a substituent or a hydrogen atom, or two or more thereof may be bonded to each other to form a ring structure. Ry¹ and Ry² each independently represent a hydrocarbon group which may have a substituent or a hydrogen atom, or may be bonded to each other to form a ring structure.

[symbol]

The doublet line of the dotted line and the straight line shown above represents a double bond or a single bond. Where valence permits, Rz¹ to Rz⁴ each independently represent a hydrocarbon group which may have a substituent or a hydrogen atom, or two or more thereof may be bonded to each other to form a ring structure. Here, Rx⁵ and Rx⁶, two or more of Rx⁷ and Rx⁸, Ry¹ and Ry², or two or more of Rz¹ to Rz⁴ are bonded to each other to form an aromatic ring. Further, at least one of Rx⁵ to Rx⁸, Ry¹, Ry², and Rz¹ to Rz⁴ contains an anion group represented by General Formula (b0-r-an1), and the entire anion moiety is an n-valent anion. In addition, at least one of Rx⁵ to Rx⁸, Ry¹, Ry², and Rz¹ to Rz⁴ contains a hydrocarbon group having a bromine atom or a hydrocarbon group having an iodine atom. n represents an integer of 1 or greater.]

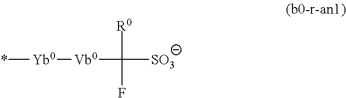

[In the formula, Yb⁰ represents a divalent linking group or a single bond. Vb⁰ represents a single bond, an alkylene group, or a fluorinated alkylene group. * represents a bonding site.]

In Formula (b0-an2), Rx⁵ and Rx⁶, Rx⁷ and Rx⁸, Ry¹ and Ry², and Rz¹ to Rz⁴ have the same definitions as Rx⁵ and Rx⁶, Rx⁷ and Rx⁸, Ry¹ and Ry², and Rz¹ to Rz⁴ in Formula (b0-an1), respectively.

In Formula (b0-an2), Rx⁵ and Rx⁶, two or more of Rx⁷'s and Rx⁸'s, Ry¹ and Ry², or two or more of Rz¹ to Rz⁴ are bonded to each other to form an aromatic ring. The aromatic ring has the same definition as in the section of General Formula (b0).

In Formula (b0-an2), at least one of Rx⁵ to Rx¹, Ry¹, Ry², and Rz¹ to Rz⁴ has an anion group represented by Formula (b0-r-an1), and the entire anion moiety is an n-valent anion. n represents an integer of 1 or greater, preferably represents 1 or 2, and more preferably 1.

In Formula (b0-an2), at least one of Rx⁵ to Rx⁸, Ry¹, Ry², and Rz¹ to Rz⁴ contains the hydrocarbon group having a bromine atom or the hydrocarbon group having an iodine atom described above. Preferable aspects of the hydrocarbon group having a bromine atom or the hydrocarbon group having an iodine atom are the same as the preferable aspects of the hydrocarbon group having a bromine atom or the hydrocarbon group having an iodine atom described in the section of General Formula (b0).

In Formulae (b0-an0), (b0-an1), and (b0-an2), it is preferable that Ry¹ and Ry² be bonded to each other to form a ring structure and more preferable that the ring structure to be formed be an aromatic hydrocarbon (an aromatic ring or an aromatic heterocyclic ring) which may have a substituent from the viewpoint of shortening the diffusion of the acid generated upon light exposure and the acid diffusion controllability.

In Formulae (b0-an0), (b0-an1), and (b0-an2), it is preferable that Rz¹ to Rz⁴ be bonded to each other to form a ring structure, where the ring structure to be formed is preferably a ring structure that shares one side (the bond between a carbon atom to which Rz¹ and Rz² are bonded and a carbon atom to which Rz³ and Rz⁴ are bonded) of the 6-membered ring in the formula and more preferably an aromatic hydrocarbon (an aromatic ring or an aromatic heterocyclic ring) which may have a substituent from the viewpoint of the diffusion controllability of the acid generated upon light exposure.

In Formulae (b0-an1) and (b0-an2), it is preferable that Rx⁷ and Rx⁵ be bonded to each other to form a ring structure and more preferable that the ring structure to be formed be an aromatic hydrocarbon (an aromatic ring or an aromatic heterocyclic ring) which may have a substituent from the viewpoint of shortening the diffusion of the acid generated upon light exposure and the acid diffusion controllability.

In Formula (b0-an2), the ring structure formed in Rx⁷ and Rx⁸ is preferably a ring structure that shares one side (the bond between the same carbon atoms to which Rx⁷ and Rx⁵ are bonded) of the 6-membered ring in the formula and more preferably an aromatic hydrocarbon (an aromatic ring or an aromatic heterocyclic ring) which may have a substituent.

In the entire anion represented by Formula (b0-an2), the number of ring structures each formed by Rx⁷ and Rx⁸, Ry¹ and Ry², and Rz¹ to Rz⁴ being bonded to each other may be one or two or more and is preferably two or three.

In the present embodiment, from the viewpoint of improving the sensitivity and CDU, an anion represented by General Formula (b0-an3) is particularly preferable as the anion moiety of the component (B0).

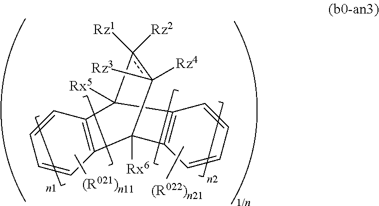

[In the formula, Rx⁵ and Rx⁶ each independently represent a hydrocarbon group which may have a substituent or a hydrogen atom. [symbol] The doublet line of the dotted line and the straight line shown above represents a double bond or a single bond. Where valence permits, Rz¹ to Rz⁴ each independently represent a hydrocarbon group which may have a substituent or a hydrogen atom, or two or more thereof may be bonded to each other to form a ring structure. Here, at least one of Rx⁵ and Rx⁶ and Rz¹ to Rz⁴ contains an anion group represented by General Formula (b0-r-an1), and the entire anion moiety is an n-valent anion. n represents an integer of 1 or greater. In addition, at least one of Rx⁵ and Rx⁶ and Rz¹ to Rz⁴ contains a hydrocarbon group having a bromine atom or a hydrocarbon group having an iodine atom. R⁰²¹ represents an alkyl group, an alkoxy group, a halogen atom, a halogenated alkyl group, a hydroxyl group, a carbonyl group, or a nitro group. n1 represents an integer of 1 to 3. n1l represents an integer of 0 to 8. R⁰²² represents an alkyl group, an alkoxy group, a halogen atom, a halogenated alkyl group, a hydroxyl group, a carbonyl group, or a nitro group. n2 represents an integer of 1 to 3. n21 represents an integer of 0 to 8.]

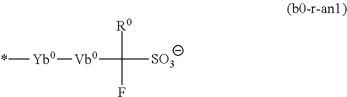

[In the formula, Yb⁰ represents a divalent linking group or a single bond. Vb⁰ represents a single bond, an alkylene group, or a fluorinated alkylene group. * represents a bonding site.]

In Formula (b0-an3), Rx⁵ and Rx⁶ and Rz¹ to Rz⁴ have the same definitions as Rx⁵ and Rx⁶ and Rz¹ to Rz⁴ in Formula (b0-an1), respectively.

In Formula (b0-an3), R⁰²¹ represents an alkyl group, an alkoxy group, a halogen atom, a halogenated alkyl group, a hydroxy group, a carbonyl group, or a nitro group.

The alkyl group as R⁰²¹ is preferably an alkyl group having 1 to 5 carbon atoms and more preferably a methyl group, an ethyl group, a propyl group, an n-butyl group, or a tert-butyl group.

The alkoxy group as R⁰²¹ is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as R⁰²¹ is preferably a fluorine atom.

Examples of the halogenated alkyl group as R⁰²¹ include a group in which some or all hydrogen atoms in an alkyl group having 1 to 5 carbon atoms such as a methyl group, an ethyl group, a propyl group, an n-butyl group, or a tert-butyl group have been substituted with the above-described halogen atom.

Among these, from the viewpoint of the compatibility with the component (A), it is preferable that R⁰²¹ represent an alkyl group, a halogen atom, or a halogenated alkyl group.

In Formula (b0-an3), n1 represents an integer of 1 to 3, preferably 1 or 2, and more preferably 1.

In Formula (b0-an3), n11 represents an integer of 0 to 8, preferably an integer of 0 to 4, more preferably 0, 1, or 2, and still more preferably 0 or 1.

In Formula (b0-an3), R⁰²²'s represent an alkyl group, an alkoxy group, a halogen atom, a halogenated alkyl group, a hydroxy group, a carbonyl group, or a nitro group, and each has the same definition as R⁰²¹ described above. Among these, from the viewpoint of the compatibility with the component (A), it is preferable that R⁰²² represent an alkyl group, a halogen atom, or a halogenated alkyl group.

In Formula (b0-an3), n2 represents an integer of 1 to 3, preferably 1 or 2, and particularly preferably 1.

In Formula (b0-an3), n21 represents an integer of 0 to 8, preferably an integer of 0 to 4, more preferably 0, 1, or 2, and particularly preferably 0 or 1.

Here, in Formula (b0-an3), at least one of Rx⁵ and Rx⁶ and Rz¹ to Rz⁴ has an anion group represented by Formula (b0-r-an1), and the entire anion moiety is an n-valent anion. n represents an integer of 1 or greater, preferably represents 1 or 2, and more preferably 1.

In Formula (b0-an3), at least one of Rx⁵ and Rx⁶ and Rz¹ to Rz⁴ contains the hydrocarbon group having a bromine atom or the hydrocarbon group having an iodine atom described above. Preferable aspects of the hydrocarbon group having a bromine atom or the hydrocarbon group having an iodine atom are the same as the preferable aspects of the hydrocarbon group having a bromine atom or the hydrocarbon group having an iodine atom described in the section of General Formula (b0).

In Formulae (b0-an0), (b0-an1), (b0-an2), and (b0-an3), it is preferable that at least one of Rz¹ to Rz⁴ contain an anion group from the viewpoint that the effects of the present invention are excellent. In a case where two or more of Rz¹ to Rz⁴ are bonded to each other to form a ring structure, a carbon atom that forms the ring structure or a hydrogen atom bonded to the carbon atom may be substituted with the above-described anion group.

In Formulae (b0-an0), (b0-an1), (b0-an2), and (b0-an3), it is preferable that at least one of Rz¹ to Rz⁴ contain the hydrocarbon group having a bromine atom or the hydrocarbon group having an iodine atom described above from the viewpoint that the effects of the present invention are excellent. In a case where two or more of Rz¹ to Rz⁴ are bonded to each other to form a ring structure, a hydrogen atom bonded to a carbon atom forming the ring structure may be substituted with the hydrocarbon group having a bromine atom or the hydrocarbon group having an iodine atom.

Specific examples of the anion moiety of the component (B0) are shown below.

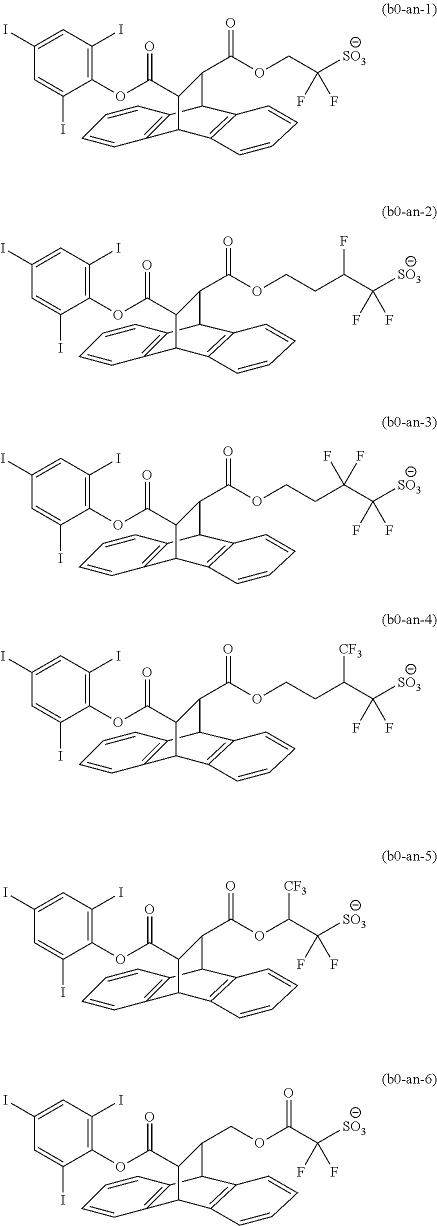

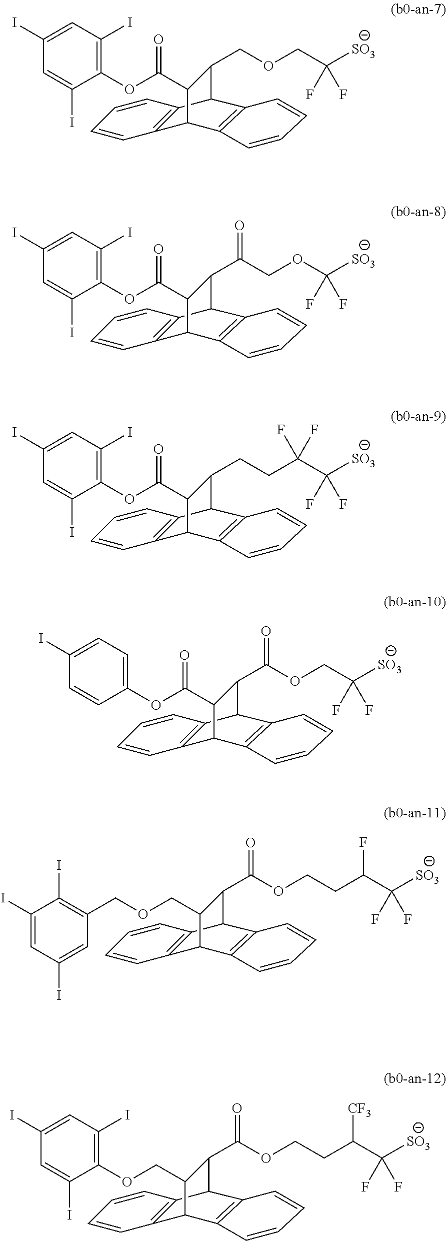

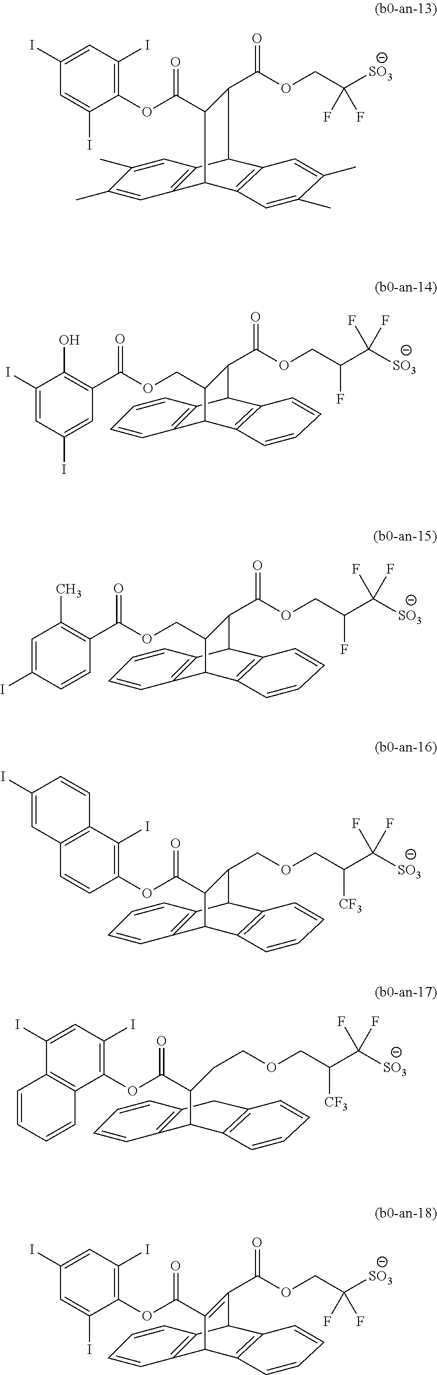

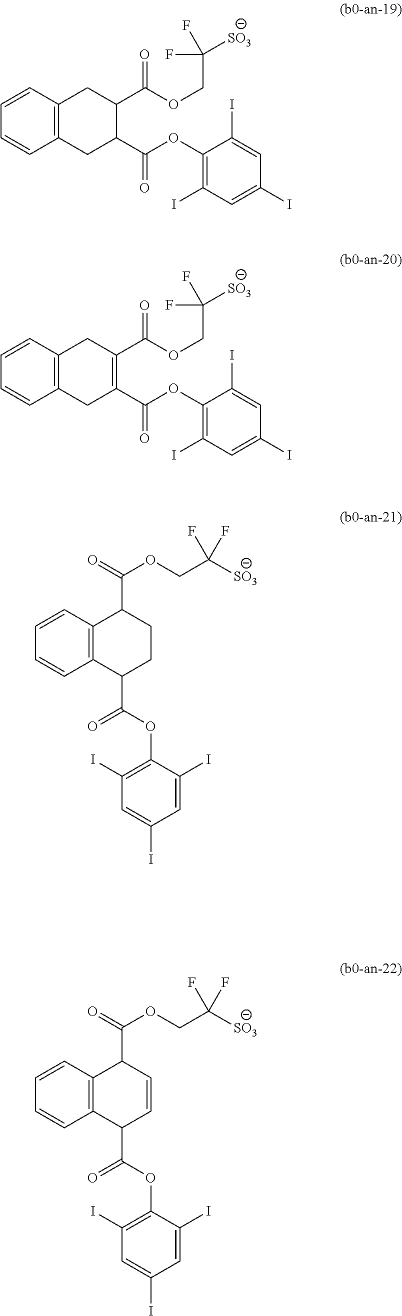

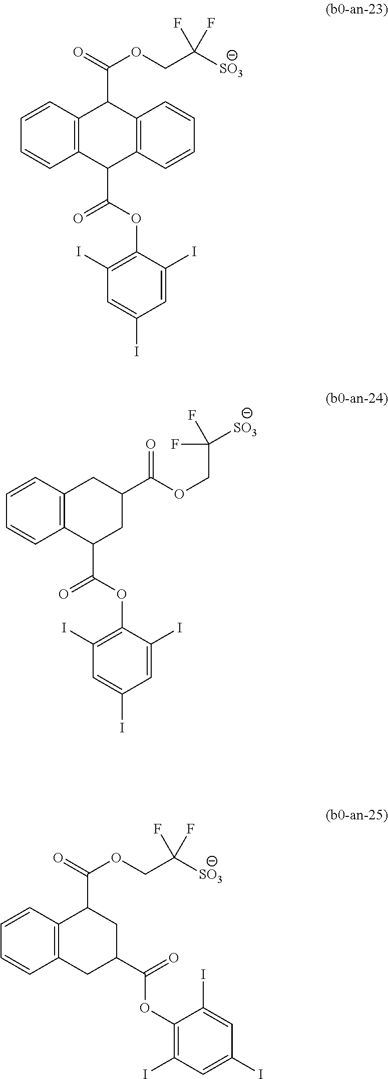

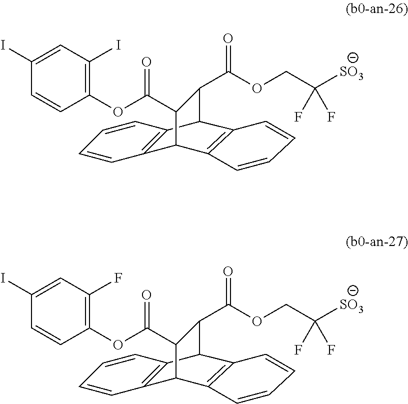

Among the examples, the anion moiety of the component (B0) is preferably an anion represented by any of Chemical Formulae (b0-an-1) to (b0-an-18), (b0-an-26), and (b0-an-27), more preferably an anion represented by any of Chemical Formulae (b0-an-1) to (b0-an-10), (b0-an-26), and (b0-an-27), and still more preferably an anion represented by any of Chemical Formulae (b0-an-1) to (b0-an-9).

### {Cation Moiety of Component (B0)}

In General Formula (b0), M^{m+} represents an m-valent organic cation. Among these, a sulfonium cation and an iodonium cation are preferable. m represents an integer of 1 or greater.

Preferred examples of the cation moiety ((M^{m+})_{1/m}) include organic cations represented by each of General Formulae (ca-1) to (ca-3).

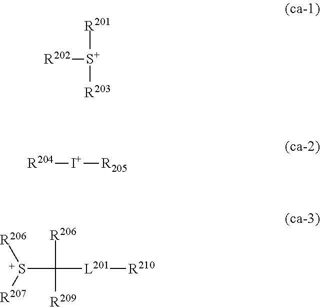

[In the formulae, R²⁰¹ to R²⁰⁷ each independently represent an aryl group, an alkyl group, or an alkenyl group, which may have a substituent. R²⁰¹ to R²⁰³, and R²⁰⁶ and R²⁰⁷ may be bonded to each other to form a ring with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms. Among these, a phenyl group or a naphthyl group is preferable.

As the alkyl group as R²⁰¹ to R²⁰⁷, a chain-like or cyclic alkyl group having 1 to 30 carbon atoms is preferable.

It is preferable that the alkenyl group as R²⁰¹ to R²⁰⁷ have 2 to 10 carbon atoms.

Examples of the substituent that R²⁰¹ to R²⁰⁷ and R²¹⁰ may have include an alkyl group, a halogen atom, a halogenated alkyl group, a carbonyl group, a cyano group, an amino group, an aryl group, and groups represented by each of General Formulae (ca-r-1) to (ca-r-7).

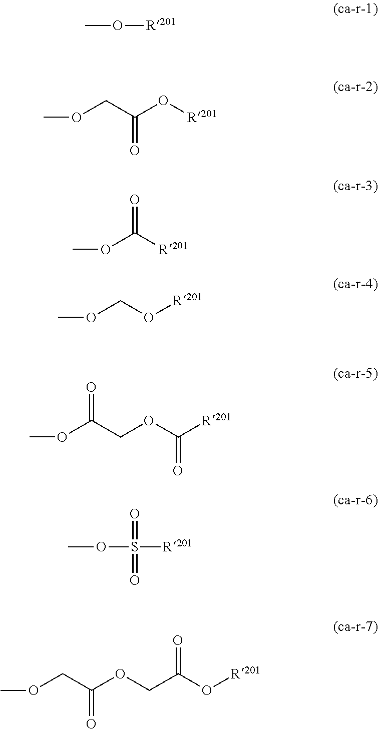

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′¹⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring-based polycyclic skeleton such as a cyclic group having a steroid skeleton are preferable as the polycycloalkane.

Among these examples, as the cyclic aliphatic hydrocarbon group as R′²¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a hetero atom such as a heterocyclic ring. Specific examples thereof include lactone-containing cyclic groups represented by each of General Formulae (a2-r-1) to (a2-r-7), —SO₂-containing cyclic groups represented by each of General Formulae (b5-r-1) to (b5-r-4), and other heterocyclic groups represented by each of Chemical Formulae (r-hr-1) to (r-hr-16).

Examples of the substituent for the cyclic group as R′²⁰ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Examples of the above-described halogenated alkyl group as the substituent include a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group. Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂-, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ have 2 to 10 carbon atoms.

As the SO₂-containing cyclic group as R²¹⁰ which may have a substituent, “—SO₂-containing polycyclic group” is preferable, and a group represented by General Formula (b5-r-1) is more preferable.

Specific examples of suitable cations represented by Formula (ca-1) include cations represented by each of Chemical Formulae (ca-1-1) to (ca-1-113).

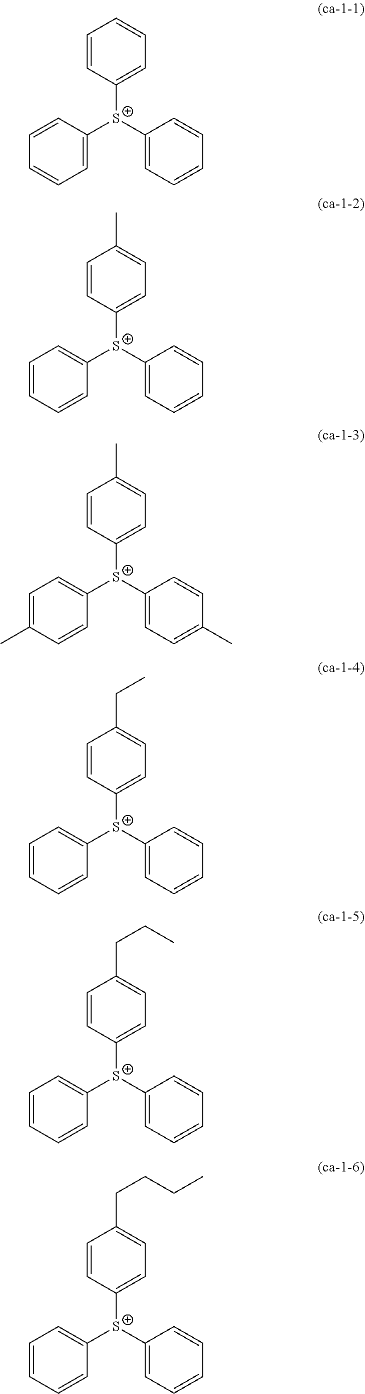

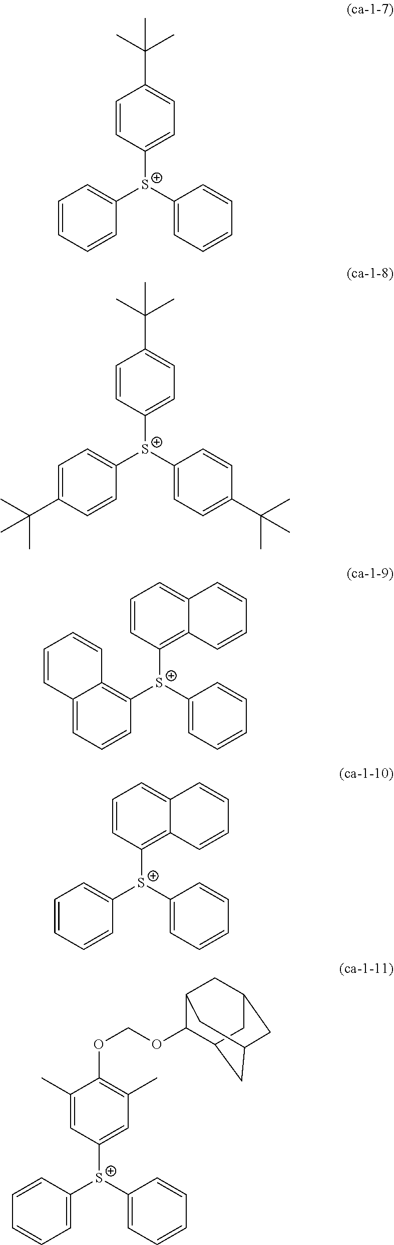 [SMILES: CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1 ; Cc1cc([S+](c2ccccc2)c2ccccc2)cc(C)c1OCOC1C2CC3CC(C2)CC1C3]

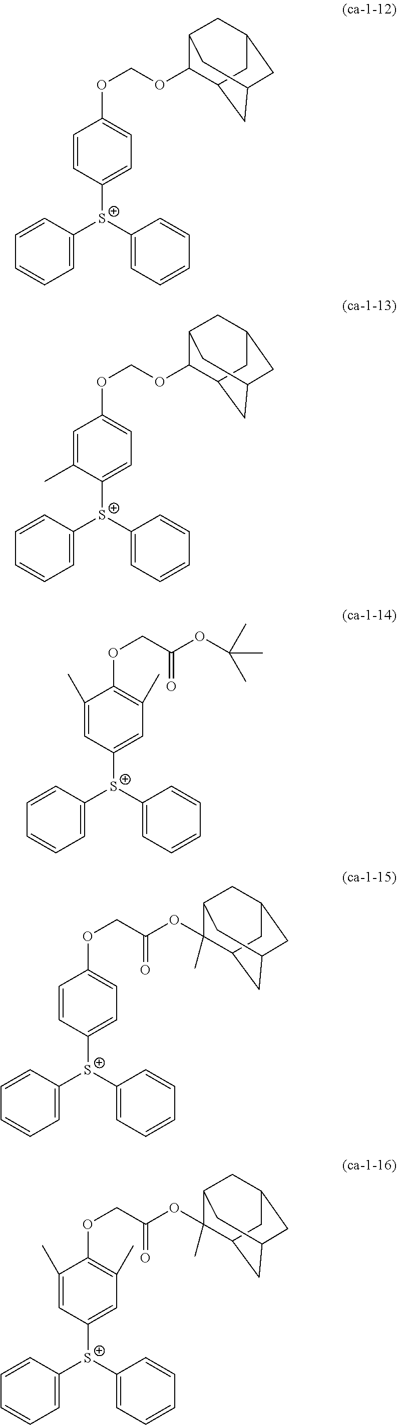 [SMILES: c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C ; CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3]

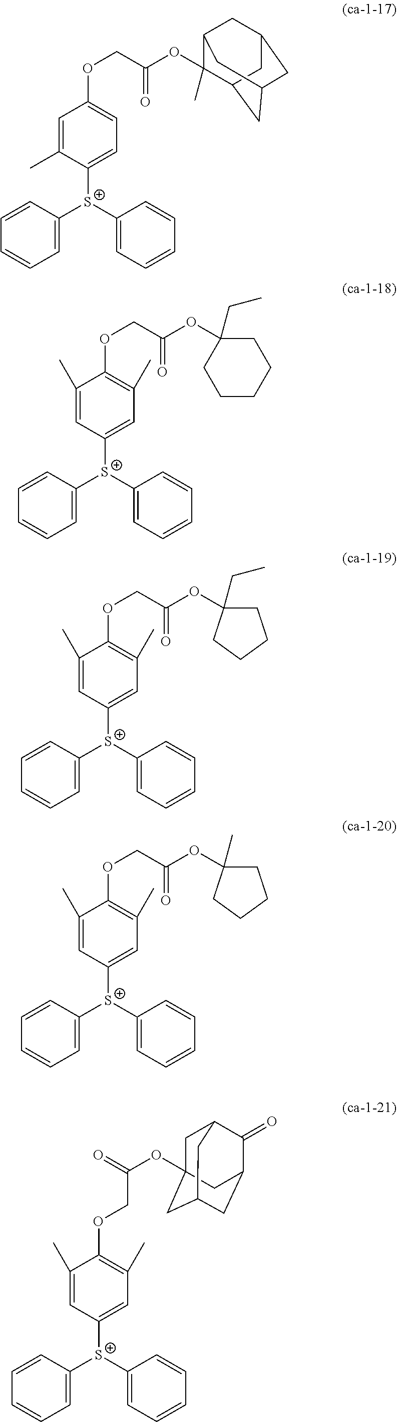 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2]

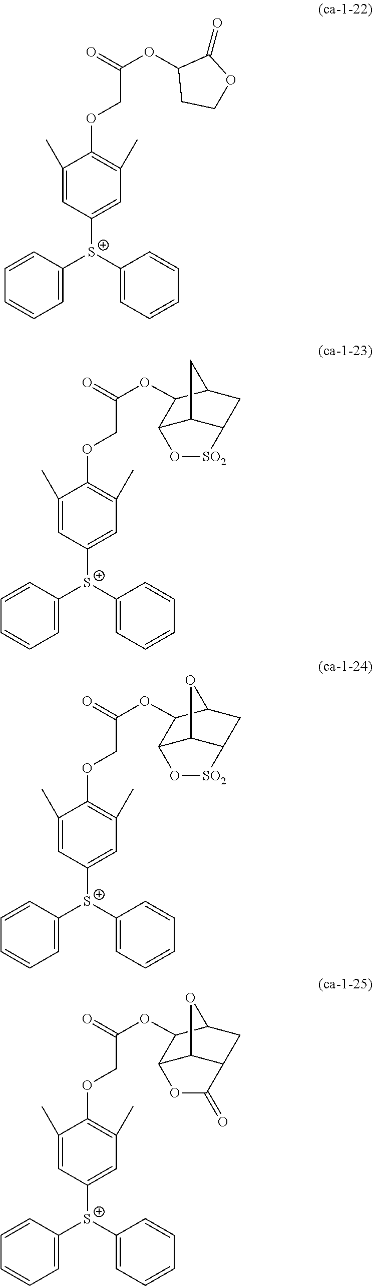 [SMILES: Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2]

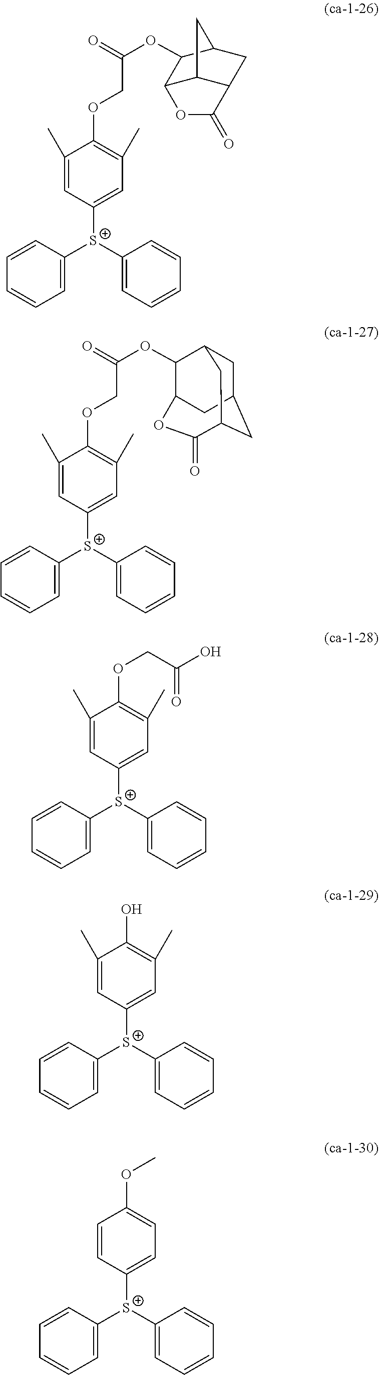 [SMILES: Cc1cc([S+](c2ccccc2)c2ccccc2)cc(C)c1OCC(=O)OC1C2CC3C(=O)OC1C3C2 ; Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1]

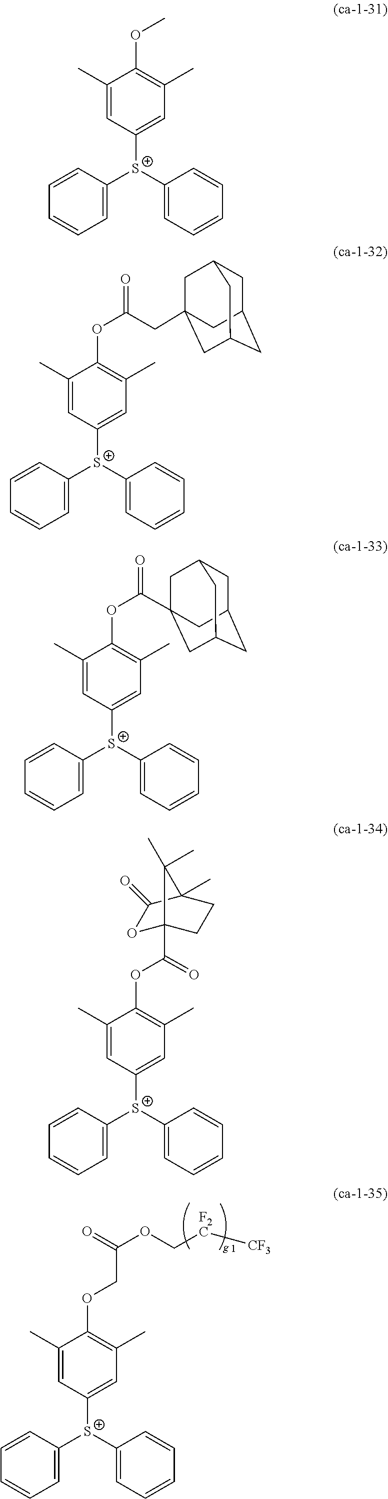

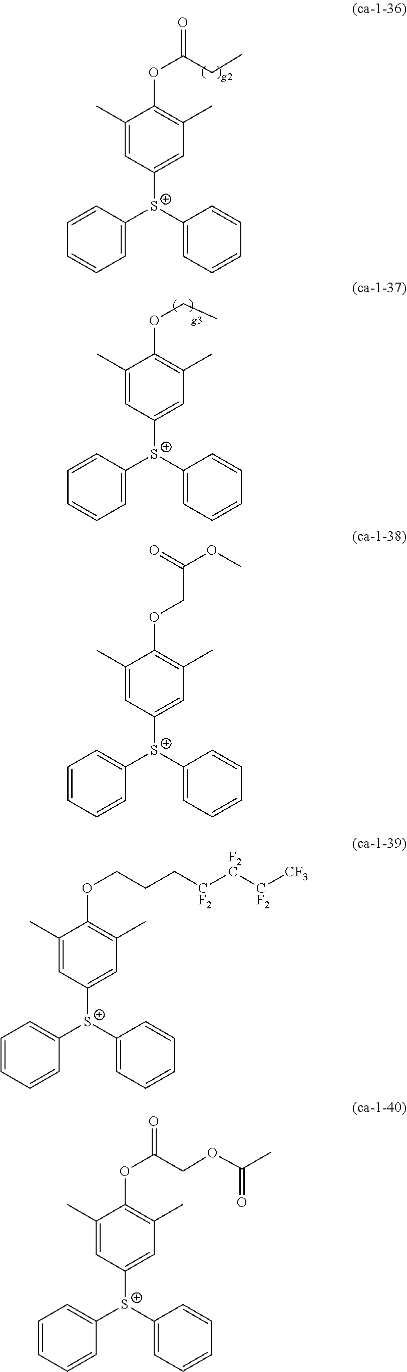

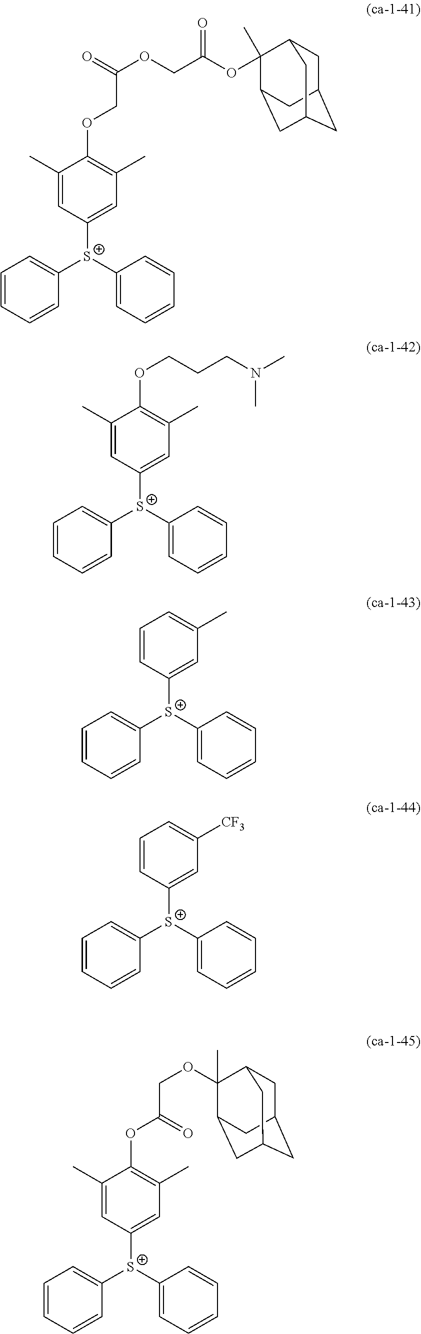 [SMILES: Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

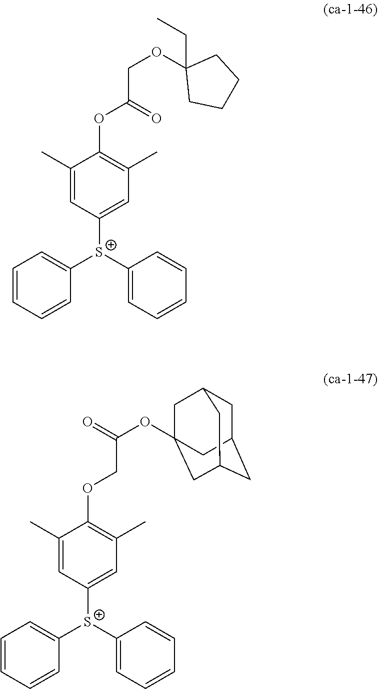 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

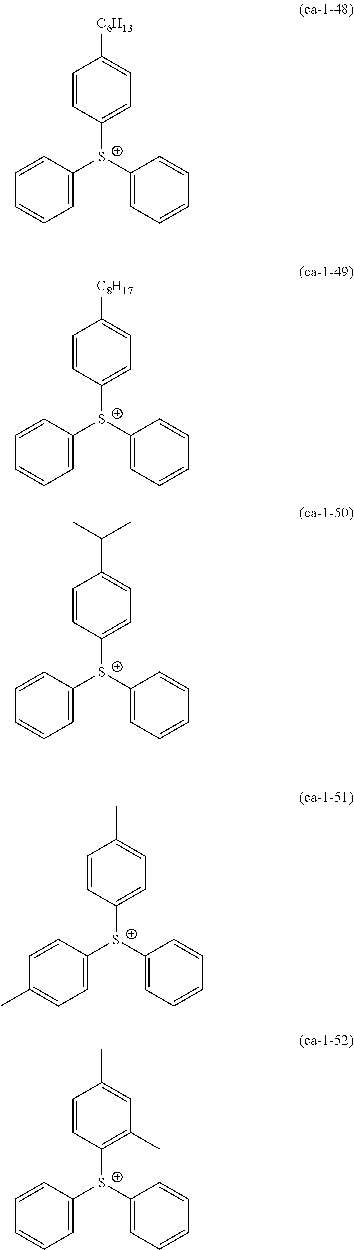

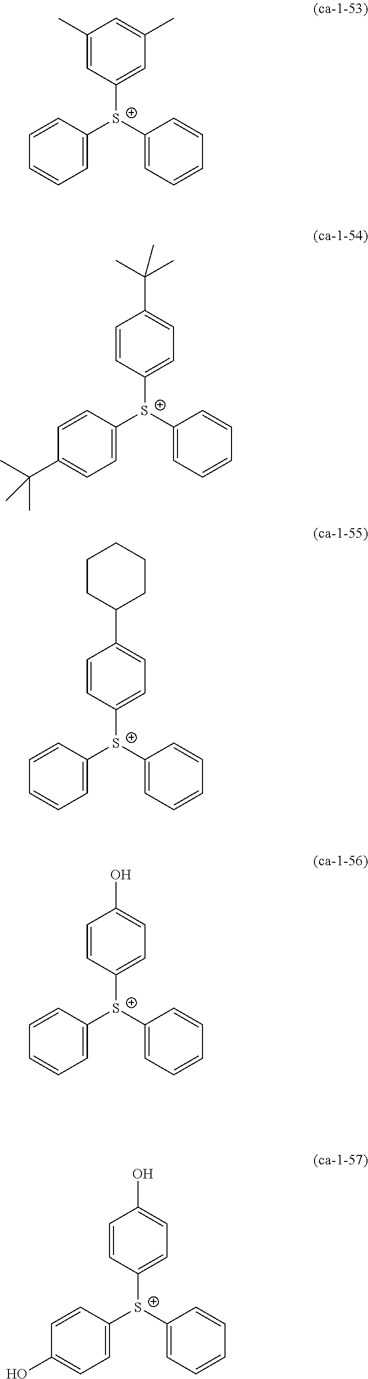 [SMILES: Cc1cc(C)cc([S+](c2ccccc2)c2ccccc2)c1 ; CC(C)(C)c1ccc([S+](c2ccccc2)c2ccc(C(C)(C)C)cc2)cc1 ; c1ccc([S+](c2ccccc2)c2ccc(C3CCCCC3)cc2)cc1 ; Oc1ccc([S+](c2ccccc2)c2ccccc2)cc1 ; Oc1ccc([S+](c2ccccc2)c2ccc(O)cc2)cc1]

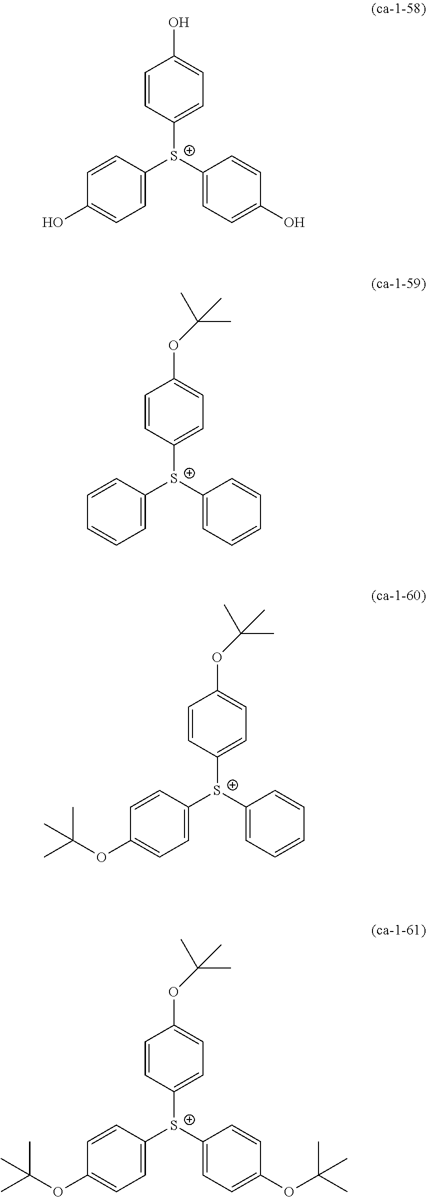 [SMILES: Oc1ccc([S+](c2ccc(O)cc2)c2ccc(O)cc2)cc1 ; CC(C)(C)Oc1ccc([S+](c2ccccc2)c2ccccc2)cc1 ; CC(C)(C)Oc1ccc([S+](c2ccccc2)c2ccc(OC(C)(C)C)cc2)cc1 ; CC(C)(C)Oc1ccc([S+](c2ccc(OC(C)(C)C)cc2)c2ccc(OC(C)(C)C)cc2)cc1]

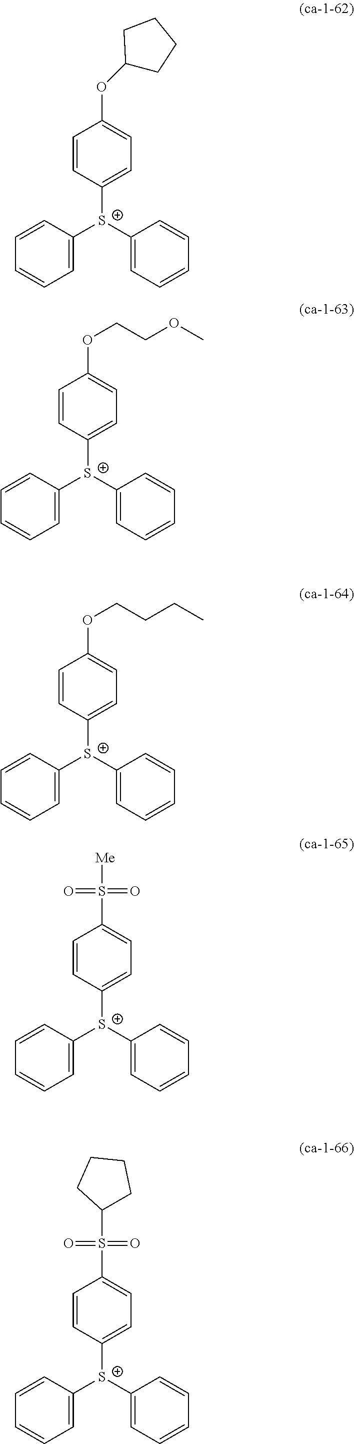 [SMILES: c1ccc([S+](c2ccccc2)c2ccc(OC3CCCC3)cc2)cc1 ; COCCOc1ccc([S+](c2ccccc2)c2ccccc2)cc1 ; CCCCOc1ccc([S+](c2ccccc2)c2ccccc2)cc1 ; CS(=O)(=O)c1ccc([S+](c2ccccc2)c2ccccc2)cc1 ; O=S(=O)(c1ccc([S+](c2ccccc2)c2ccccc2)cc1)C1CCCC1]

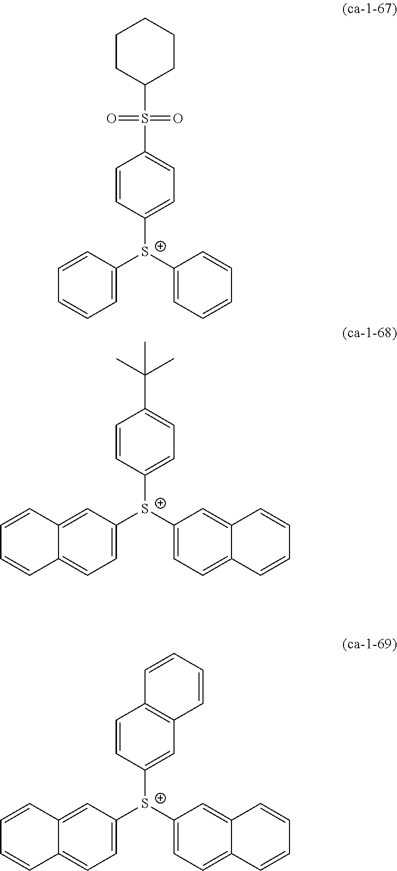 [SMILES: O=S(=O)(c1ccc([S+](c2ccccc2)c2ccccc2)cc1)C1CCCCC1 ; CC(C)(C)c1ccc([S+](c2ccc3ccccc3c2)c2ccc3ccccc3c2)cc1 ; c1ccc2cc([S+](c3ccc4ccccc4c3)c3ccc4ccccc4c3)ccc2c1]

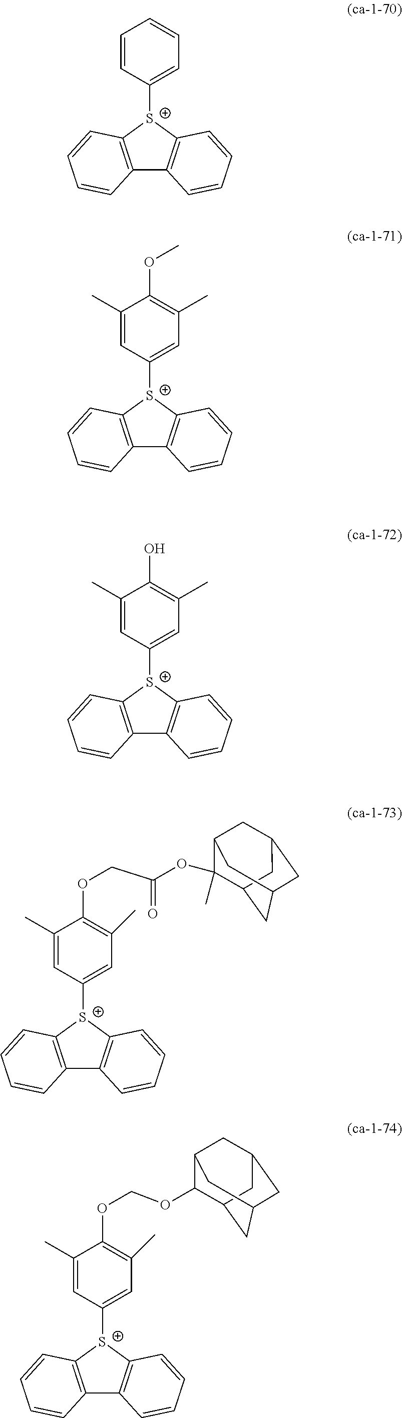 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

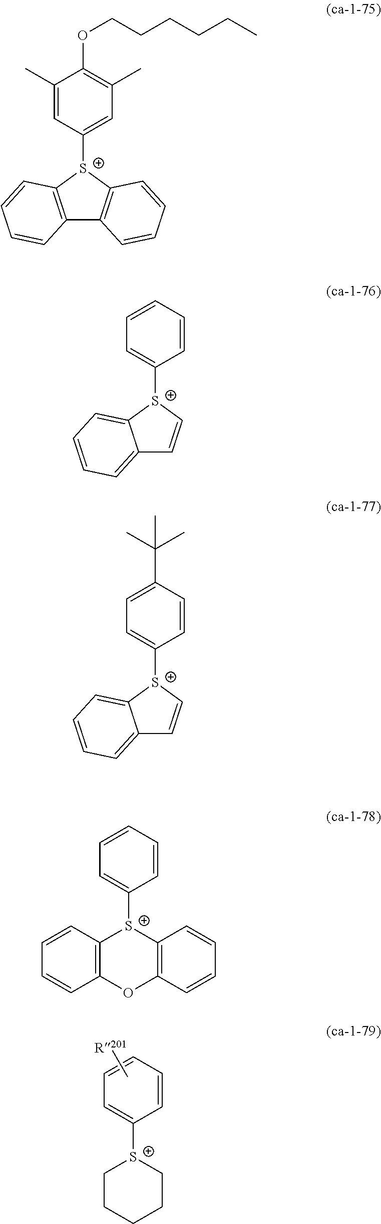

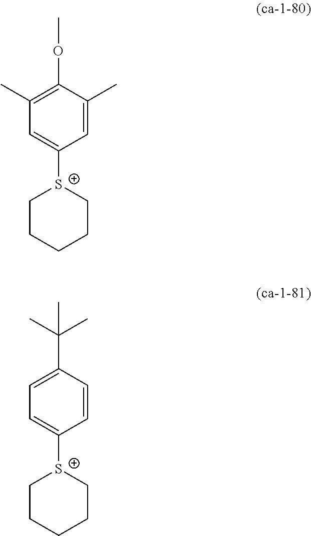

[In the formulae, R″²⁰¹ represents a hydrogen atom or a substituent, and examples of the substituent include the same groups as those for the substituents that R²⁰¹ to R²⁰¹ and R²¹⁰ to R²¹² may have.]

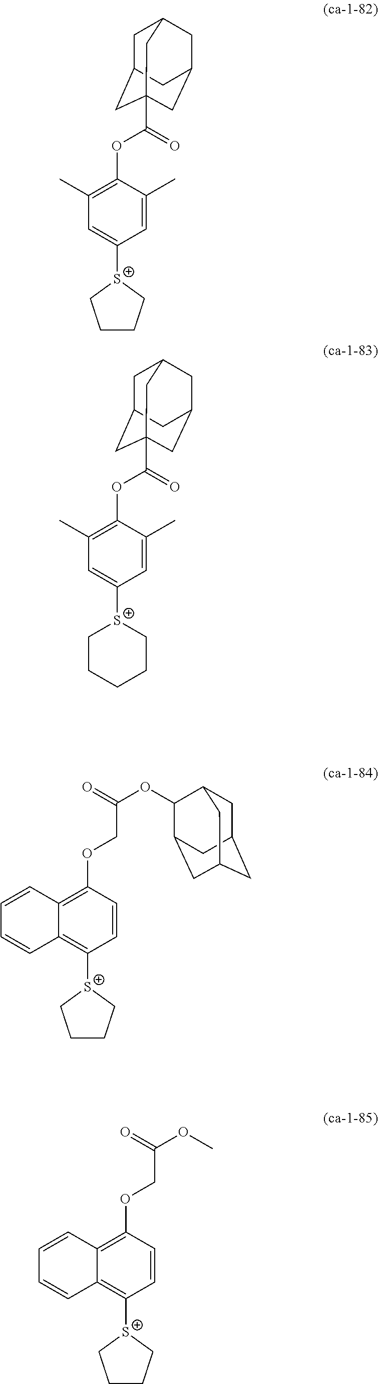 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

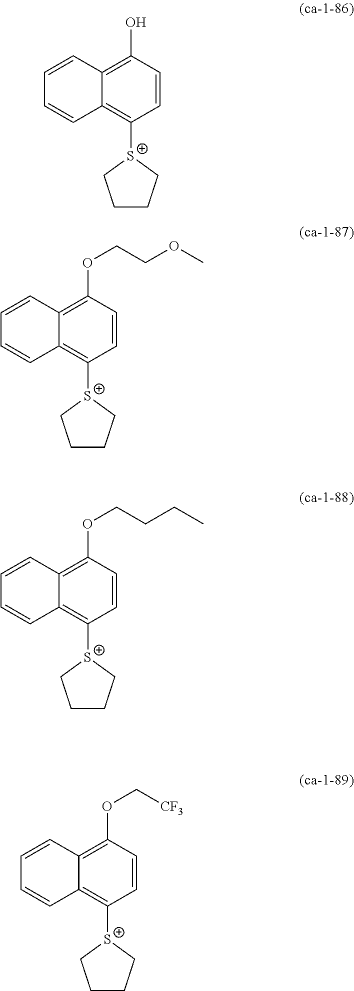 [SMILES: Oc1ccc([S+]2CCCC2)c2ccccc12 ; COCCOc1ccc([S+]2CCCC2)c2ccccc12 ; CCCCOc1ccc([S+]2CCCC2)c2ccccc12 ; FC(F)(F)COc1ccc([S+]2CCCC2)c2ccccc12]

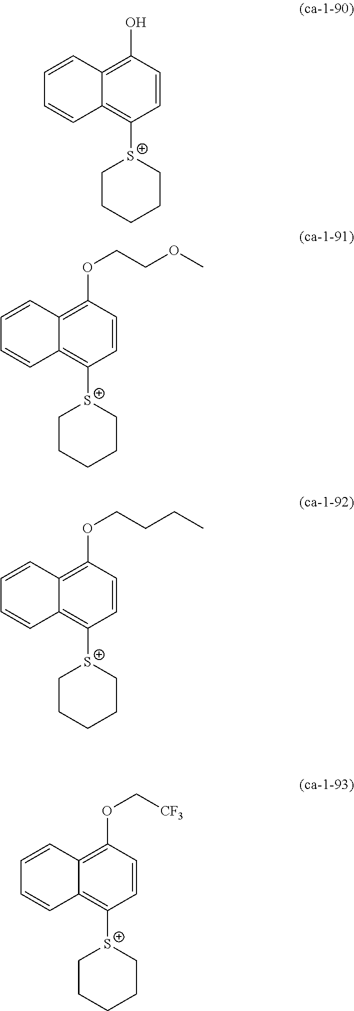 [SMILES: Oc1ccc([S+]2CCCCC2)c2ccccc12 ; COCCOc1ccc([S+]2CCCCC2)c2ccccc12 ; CCCCOc1ccc([S+]2CCCCC2)c2ccccc12 ; FC(F)(F)COc1ccc([S+]2CCCCC2)c2ccccc12]

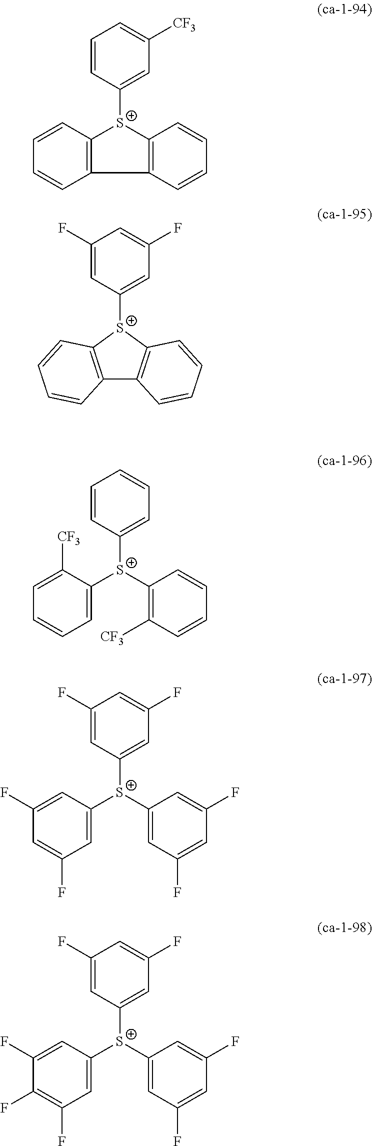 [SMILES: FC(F)(F)c1cccc(-[s+]2c3ccccc3c3ccccc32)c1 ; Fc1cc(F)cc(-[s+]2c3ccccc3c3ccccc32)c1 ; FC(F)(F)c1ccccc1[S+](c1ccccc1)c1ccccc1C(F)(F)F ; Fc1cc(F)cc([S+](c2cc(F)cc(F)c2)c2cc(F)cc(F)c2)c1 ; Fc1cc(F)cc([S+](c2cc(F)cc(F)c2)c2cc(F)c(F)c(F)c2)c1]

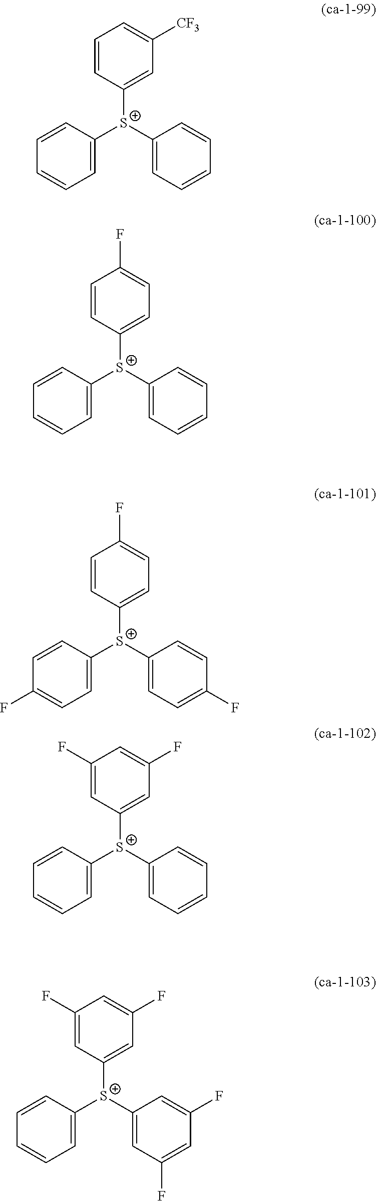 [SMILES: FC(F)(F)c1cccc([S+](c2ccccc2)c2ccccc2)c1 ; Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1]

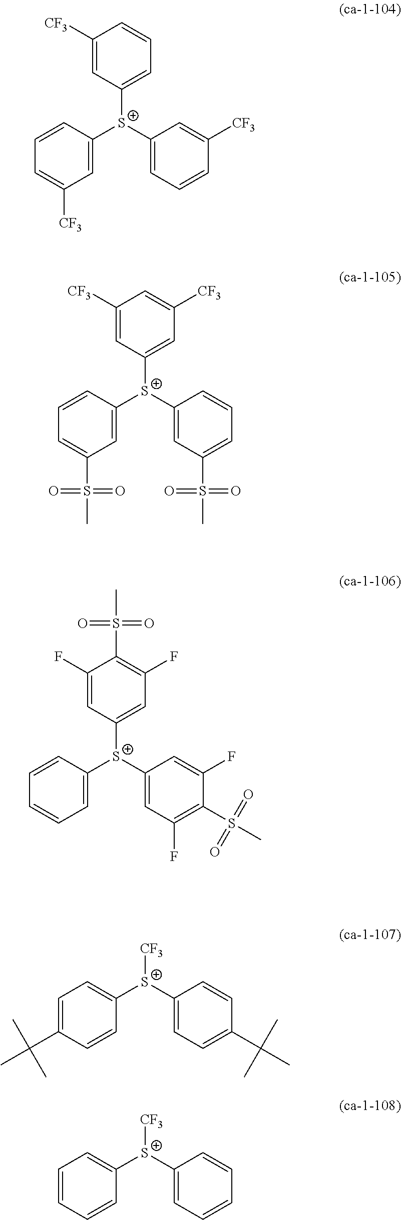

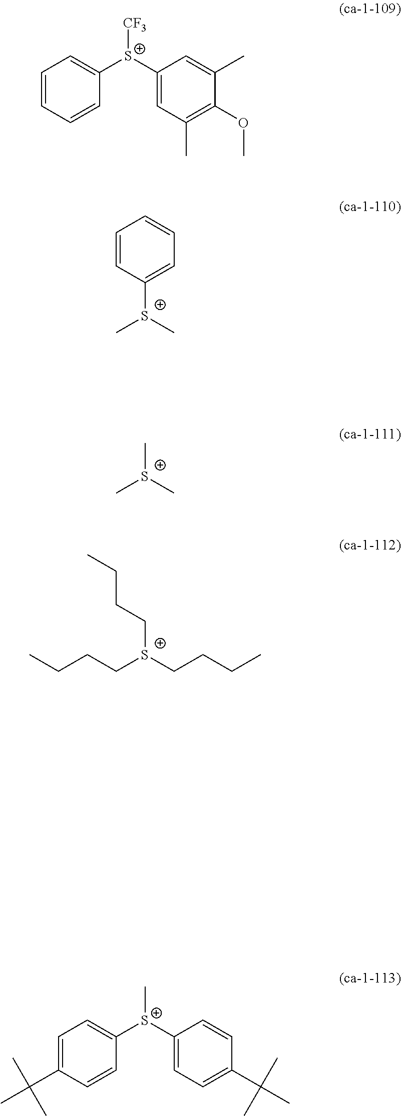

Specific examples of suitable cations represented by Formula (ca-2) include a diphenyliodonium cation and a bis(4-tert-butylphenyl)iodonium cation.

Specific examples of suitable cations represented by Formula (ca-3) include cations represented by each of Formulae (ca-3-1) to (ca-3-6).

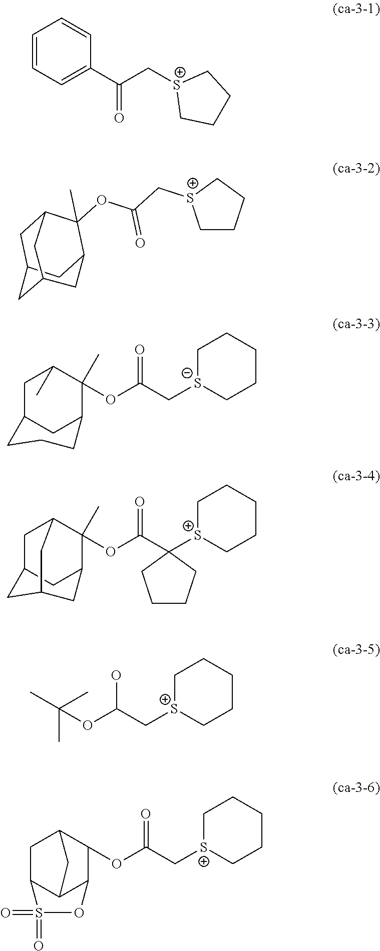 [SMILES: O=C(C[S+]1CCCC1)c1ccccc1 ; CC1(OC(=O)C[S+]2CCCC2)C2CC3CC(C2)CC1C3 ; CC1CC2CCCC(C2)C1(C)OC(=O)C[S-]1CCCCC1 ; CC1(OC(=O)C2([S+]3CCCCC3)CCCC2)C2CC3CC(C2)CC1C3 ; CC(C)(C)OC(O)C[S+]1CCCCC1 ; O=C(C[S+]1CCCCC1)OC1C2CC3C1OS(=O)(=O)C3C2]

Among the examples, a cation represented by General Formula (ca-1) is preferable as the cation moiety ((M^{m+})_{1/m}).

Further, from the viewpoint of improving the decomposability of the cation moiety, it is preferable that in the cation represented by General Formula (ca-1), R²⁰¹ to R²⁰³ each independently represent an aryl group which may have a substituent and contain at least one electron-withdrawing group as the substituent or R²⁰¹ to R²⁰³ each independently represent an aryl group which may have a substituent and any two of R²⁰¹ to R²⁰³ be bonded to each other to form a ring with the sulfur atom in the formula and more preferable that in the cation represented by General Formula (ca-1), R²⁰¹ to R²⁰³ each independently represent an aryl group which may have a substituent and contain at least one electron-withdrawing group as the substituent.

The electron-withdrawing group may be used alone or two or more kinds thereof may be used.

Further, the electron-withdrawing group may be a monovalent electron-withdrawing group or a divalent electron-withdrawing group.

Specific examples of the electron-withdrawing group include an acyl group, a halogen atom, a halogenated alkyl group, a halogenated alkoxy group, a halogenated aryloxy group, a halogenated alkylamino group, a halogenated alkylthio group, a cyano group, a nitro group, a dialkylphosphono group, a diarylphosphono group, an alkylsulfonyl group, a cycloalkylsulfonyl group, an arylsulfonyl group, a sulfonyloxy group, an acylthio group, a sulfamoyl group, a thiocyanate group, and a thiocarbonyl group.

Among these, from the viewpoint of increasing the sensitivity, the electron-withdrawing group is preferably a fluorine atom, a fluorinated alkyl group, or a cycloalkylsulfonyl group, more preferably a fluorine atom or a cycloalkylsulfonyl group, and still more preferably a fluorine atom.

The cation moiety ((M^{m+})_{1/m}) is preferably a cation represented by any of Chemical Formulae (ca-1-65) to (ca-1-67), (ca-1-70), and (ca-1-94) to (Ca-1-106), more preferably a cation represented by any of Chemical Formulae (ca-1-67), (ca-1-70), and (ca-I-103), and still more preferably a cation represented by Chemical Formula (ca-1-103).

In the resist composition according to the present embodiment, among the examples, it is preferable that the component (B0) be a compound represented by General Formula (b0-1).

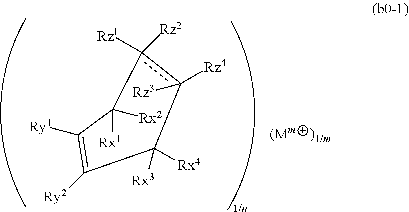

[In the formula, Rx¹ to Rx⁴ each independently represent a hydrocarbon group which may have a substituent or a hydrogen atom, or two or more thereof may be bonded to each other to form a ring structure. Ry¹ and Ry² each independently represent a hydrocarbon group which may have a substituent or a hydrogen atom, or may be bonded to each other to form a ring structure.

[symbol]

The doublet line of the dotted line and the straight line shown above represents a double bond or a single bond. Where valence permits, Rz¹ to Rz⁴ each independently represent a hydrocarbon group which may have a substituent or a hydrogen atom, or two or more thereof may be bonded to each other to form a ring structure. Here, at least one of two or more of Rx¹ to Rx⁴, Ry¹ and Ry², and two or more of Rz¹ to Rz⁴ are bonded to each other to form an aromatic ring. Further, at least one of Rx¹ to Rx⁴, Ry¹ and Ry², and Rz¹ to Rz⁴ has an anion group represented by General Formula (b0-r-an1), and the entire anion moiety is an n-valent anion. Further, at least one of Rx¹ to Rx⁴, Ry¹ and Ry², and Rz¹ to Rz⁴ contains a hydrocarbon group having a bromine atom or a hydrocarbon group having an iodine atom. n represents an integer of 1 or greater. m represents an integer of 1 or greater, and M^{m+} represents an m-valent organic cation.]

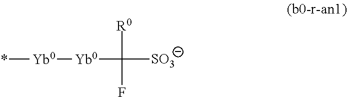

[In the formula, Yb⁰ represents a divalent linking group or a single bond. Vb⁰ represents a single bond, an alkylene group, or a fluorinated alkylene group. R⁰ represents a fluorinated alkyl group having 1 to 5 carbon atoms or a fluorine atom. * represents a bonding site.]

The anion moiety of the compound represented by General Formula (b0-1) is the same as the anion represented by General Formula (b0-an0).

The cation moiety of the compound represented by General Formula (b0-1) is the same as the cation moiety of the compound represented by General Formula (b0). Among these, a cation represented by General Formula (ca-1) is preferable.

Further, from the viewpoint of improving the decomposability of the cation moiety, it is preferable that in the cation represented by General Formula (ca-1), R²⁰¹ to R²⁰³ each independently represent an aryl group which may have a substituent and contain at least one electron-withdrawing group as the substituent or R²⁰¹ to R²⁰³ each independently represent an aryl group which may have a substituent and any two of R²⁰¹ to R²⁰³ be bonded to each other to form a ring with the sulfur atom in the formula and more preferable that in the cation represented by General Formula (ca-I), R⁷⁰¹ to R²⁰³ each independently represent an aryl group which may have a substituent and contain at least one electron-withdrawing group as the substituent.

Specific examples of the component (B0) are shown below but are not limited thereto.

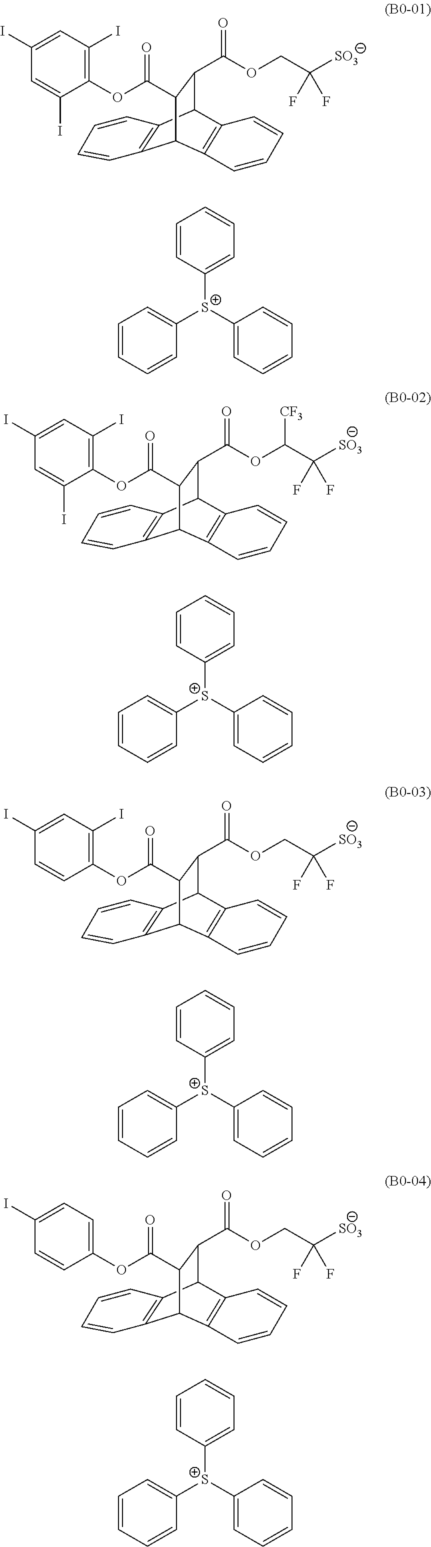

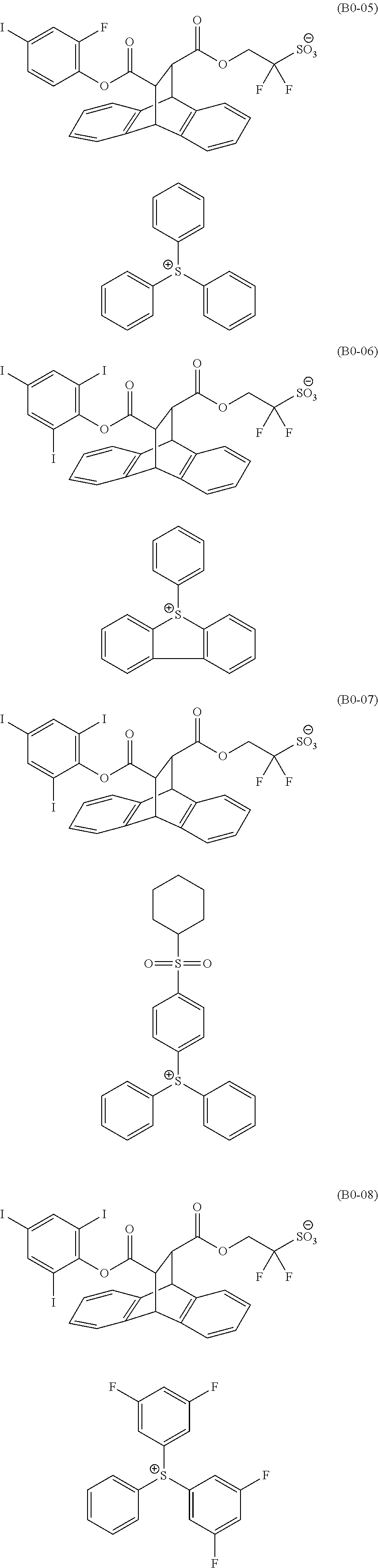

In the resist composition according to the present embodiment, the component (B0) may be used alone or may be used in a combination of two or more kinds thereof.

In the resist composition according to the present embodiment, the content of the component (B0) is preferably in a range of 5 to 60 parts by mass, more preferably in a range of 10 to 55 parts by mass, still more preferably in a range of 15 to 50 parts by mass, and particularly preferably in a range of 20 to 45 parts by mass with respect to 100 parts by mass of the component (A).

In a case where the content of the component (B0) is greater than or equal to the lower limits of the above-described preferable ranges, the lithography characteristics such as sensitivity, resolution performance, CDU, a linewise roughness (LWR) reduction property, and a shape are further improved in the resist pattern formation. Meanwhile, in a case where the content thereof is less than or equal to the upper limits of the above-described preferable ranges, a uniform solution is easily obtained, and the storage stability of the resist composition is further improved in the case of dissolving each component of the resist composition in an organic solvent.

The proportion of the component (B0) in the total component (B) contained in the resist composition according to the present embodiment is, for example, 50% by mass or greater, preferably 70% by mass or greater, and more preferably 95% by mass or greater. In addition, the proportion thereof may be 100% by mass.

The component (B) in the resist composition according to the present embodiment may contain an acid generator component (B1) other than the above-described component (B0) (hereinafter, also referred to as “component (B1)”).

### <<Component (B1)>>

Examples of the component (B1) are numerous and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators; iminosulfonate-based acid generators; and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

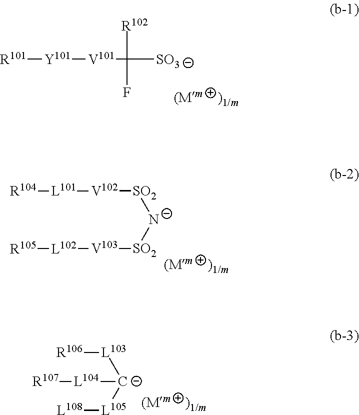

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring-based polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰ may have a hetero atom such as a heterocyclic ring. Specific examples thereof include lactone-containing cyclic groups represented by each of General Formulae (a2-r-1) to (a2-r-7), —SO₂-containing cyclic groups represented by each of General Formulae (b5-r-1) to (b5-r-4), and other heterocyclic groups represented by each of Chemical Formulae (r-hr-1) to (r-hr-16). In the formulae, * represents a bonding site bonded to Y¹⁰¹ in Formula (b-1).

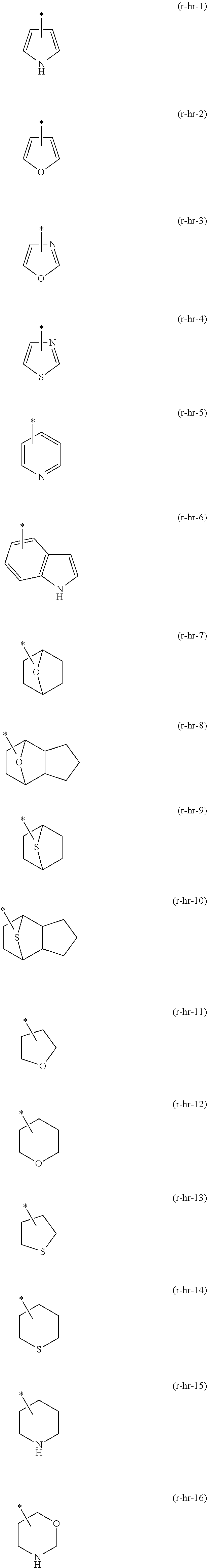

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Examples of the above-described halogenated alkyl group as the substituent include a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms. The carbonyl group as the substituent is a group that substitutes a methylene group (—CH₂—) constituting the cyclic hydrocarbon group.

The cyclic hydrocarbon group as R¹⁰¹ may be a fused cyclic group having a fused ring in which an aliphatic hydrocarbon ring and an aromatic ring are fused. Examples of the fused ring include those obtained by fusing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the fused cyclic group, a group having a fused ring in which two or three aromatic rings are fused with a bicycloalkane is preferable, and a group having a fused ring in which two or three aromatic rings are fused with bicyclo[2.2.2]octane is more preferable. Specific examples of the fused cyclic group as R¹⁰¹ include those represented by Formulae (r-br-1) and (r-br-2). Here, * in the formulae represents a bonding site bonded to Y¹⁰¹ in Formula (b-1).

Examples of the substituent that the fused cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the fused cyclic group include those exemplified as the substituent of the cyclic group as R¹⁰¹.

Examples of the aromatic hydrocarbon group as the substituent of the fused cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the fused cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a I-methylpropenyl group, and a 2-methylpropenyl group. Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent. More specifically, as the cyclic hydrocarbon group, a phenyl group, a naphthyl group, or a group in which one or more hydrogen atoms have been removed from a polycycloalkane, a lactone-containing cyclic group represented by any of General Formulae (a2-r-l) to (a2-r-7), or a —SO₂-containing cyclic group represented by any of General Formulae (b5-r-1) to (b5-r-4) is preferable, a group in which one or more hydrogen atoms have been removed from a polycycloalkane or a —SO₂-containing cyclic group represented by any of General Formulae (b5-r-1) to (b5-r-4) is more preferable, and an adamantyl group or a —SO₂-containing cyclic group represented by General Formula (b5-r-1) is still more preferable.

In Formula (b-1), Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of divalent linking groups containing an oxygen atom include linking groups represented by each of General Formulae (y-a1-1) to (y-a1-7) described above. In this case, V′¹⁰¹ in General Formulae (y-a1-1) to (y-a1-7) is bonded to R¹⁰¹ in Formula (b-i).

Y¹⁰ represents preferably a divalent linking group having an ester bond or a divalent linking group having an ether bond and more preferably a linking group represented by any of Formulae (y-a1-1) to (y-a1-5).

In Formula (b-1), V¹⁰¹ represents a single bond, an alkylene group, or a fluorinated alkylene group. It is preferable that the alkylene group and the fluorinated alkylene group as V¹⁰ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among these examples, it is preferable that V¹⁰¹ represent a single bond or a fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific examples of the anion moiety represented by Formula (b-1) include fluorinated alkyl sulfonate anions such as a trifluoromethanesulfonate anion and a perfluorobutanesulfonate anion.
Anions in component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decrease within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms be as large as possible from the viewpoint that the acid strength increases and the transparency to high energy light or electron beams having a wavelength of 250 nm or less is improved. The proportion of fluorine atoms in the chain-like alkyl group, that is, the fluorination ratio, is preferably in a range of 70% to 100% and more preferably in a range of 90% to 100%, and it is most preferable that the chain-like alkyl group be a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-I) is preferable.

### {Cation Moiety}

In Formulae (b-1), (b-2), and (b-3), M′^{m+} represents an m-valent onium cation. Among these, a sulfonium cation and an iodonium cation are preferable.

m represents an integer of 1 or greater.

Preferred examples of the cation moiety ((M′^{m+})_{1/m}) include organic cations represented by each of General Formulae (ca-I) to (ca-3).

In the resist composition according to the present embodiment, the component (B1) may be used alone or in a combination of two or more kinds thereof.

In a case where the resist composition contains the component (BI), the content of the component (B1) in the resist composition is preferably less than 40 parts by mass, more preferably in a range of 1 to 30 parts by mass, and still more preferably in a range of 1 to 20 parts by mass with respect to 100 parts by mass of the component (A).

In a case where the content of the component (B1) is set to be in the above-described preferable ranges, pattern formation can be sufficiently carried out. Further, it is preferable that each component of the resist composition be dissolved in an organic solvent from the viewpoint that a uniform solution is easily obtained and the storage stability of the resist composition is improved.

### <Other Components>

The resist composition according to the present embodiment may further contain other components in addition to the component (A) and the component (B) described above. Examples of other components include a component (D), a component (E), a component (F), and a component (S) described below.

<<Base component (D)>>

It is preferable that the resist composition according to the present embodiment further contain a base component (hereinafter, also referred to as “component (D)”) that traps (that is, controls the acid diffusion of) an acid that is generated upon light exposure, in addition to the component (A) and the component (B). The component (D) acts as a quencher (an acid diffusion control agent) which traps the acid generated in the resist composition upon light exposure.

Examples of the component (D) include a photodecomposable base (D1) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (the component (D1)) is preferable since it makes it easy to enhance the roughness reducing property. Further, in a case where the component (D1) is contained, both characteristics of increasing the sensitivity and suppressing the occurrence of coating defects are likely to be enhanced.

### In Regard to Component (D1)

In a case where a resist composition containing the component (D1) is obtained, the contrast between an exposed portion and an unexposed portion of the resist film can be further improved in the case of forming a resist pattern.

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

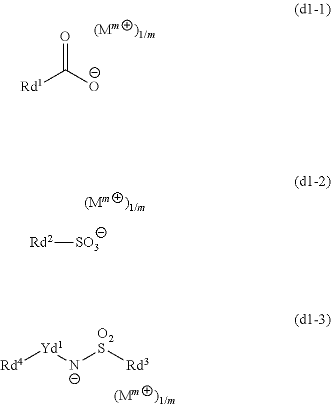

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represent an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contains a linking group represented by any of General Formulae (y-a1-1) to (y-a1-7) as a substituent, V′¹⁰¹ in General Formulae (y-a1-1) to (y-a1-7) is bonded to the carbon atom constituting the aromatic hydrocarbon group, the aliphatic cyclic group, or the chain-like alkyl group as Rd¹ in Formula (d3-1), in General Formulae (y-a1-1) to (y-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group have 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-I) are described below.

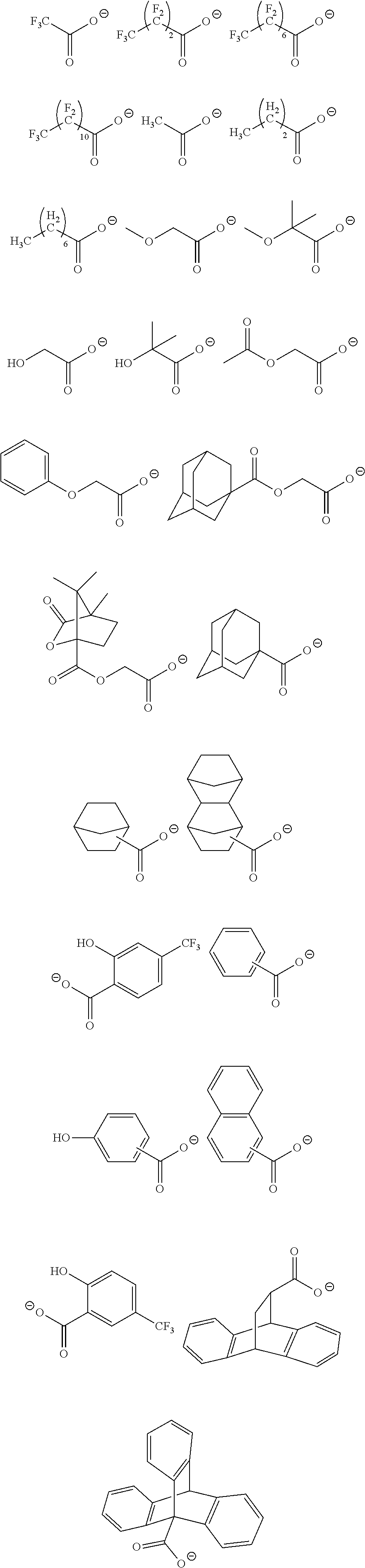

### Cation Moiety

In Formula (d1-1), M^{m+} represents an m-valent organic cation.

Suitable examples of the organic cation as M^{m+} include the same cations as those for the cations represented by each of General Formulae (ca-1) to (ca-3). Among these, a cation represented by General Formula (ca-1) is more preferable, and a cation represented by any of Formulae (ca-1-1) to (ca-1-113) is still more preferable.

The component (d1-1) may be used alone or in a combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group has preferably 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Among the examples, the anion moiety of the component (d1-2) is preferably a camphorsulfonic acid anion.

Specific preferred examples of the anion moiety in the component (d1-2) are described below.

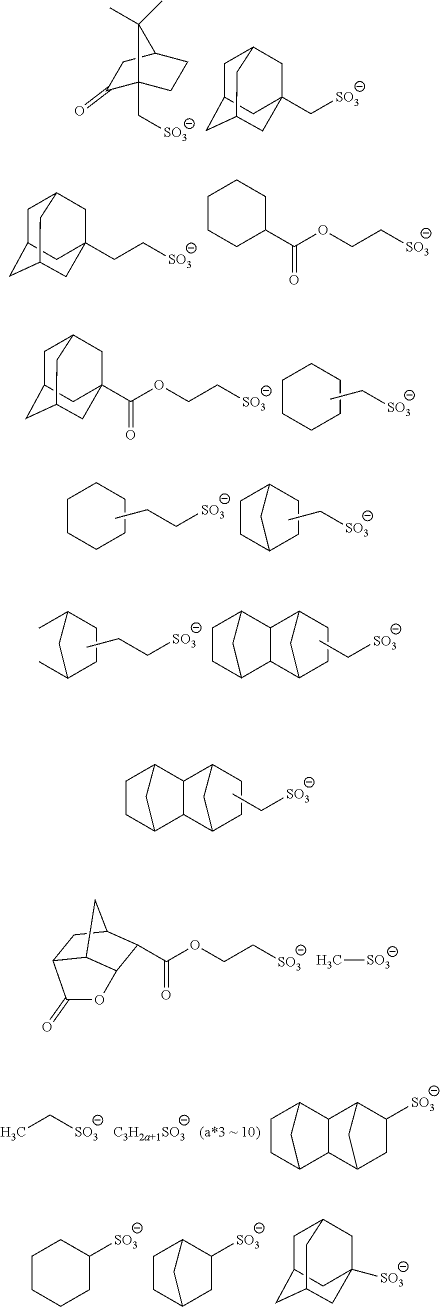

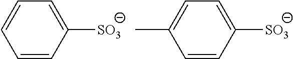

### Cation Moiety

In Formula (d1-2), M^{m+} represents an m-valent organic cation and has the same definition as M^{m+} in Formula (d1-1).

The component (d1-2) may be used alone or in a combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ be a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ be an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may further contain an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a hetero atom. Examples of the divalent linking groups are the same as those for the divalent hydrocarbon group which may have a substituent and the divalent linking group having a hetero atom described in the section of the divalent linking group as Ya²t in Formula (a2-1).

It is preferable that Yd¹ represent a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

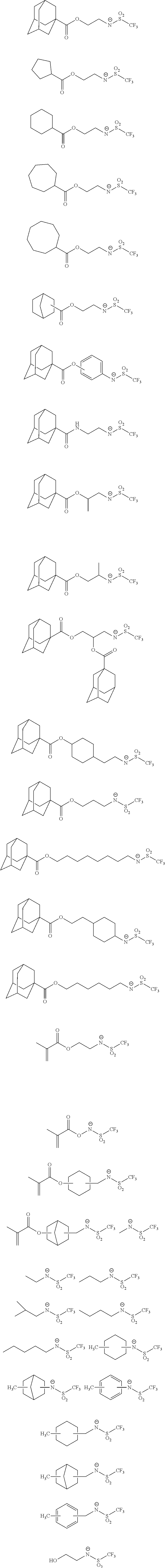

### Cation Moiety

In Formula (d1-3), M^{m+} represents an m-valent organic cation and has the same definition as M^{m+} in Formula (d1-1).

The component (d1-3) may be used alone or in a combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A1).

In a case where the content of the component (D1) is greater than or equal to the lower limits of the above-described preferable ranges, excellent lithography characteristics and an excellent resist pattern shape are easily obtained. On the other hand, in a case where the content is less than or equal to the upper limits of the above-described ranges, the sensitivity can be satisfactorily maintained and the throughput is also excellent.

In the resist composition according to the present embodiment, it is preferable that the component (DI) contain the component (d1-1).

The content of the component (d1-1) in the total component (D) contained in the resist composition according to the present embodiment is preferably 50% by mass or greater, preferably 70% by mass or greater, and still more preferably 90% by mass or greater, and the component (D) may consist of only the component (d1-1).

### Method of Producing Component (D1):

The methods of producing the component (d1- 1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 5 to 10 carbon atoms is preferable, and tri-n-pentylamine and tri-n-octylamine are particularly preferable.

Examples of the cyclic amine include a heterocyclic compound having a nitrogen atom as a hetero atom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

It is preferable that the aliphatic polycyclic amine have 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl)amine, tris{2-(1-methoxyethoxy)ethyl}amine, tris{2-(1-ethoxyethoxy)ethyl}amine, tris[2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

Among the examples, the component (D2) is preferably an alkylamine and more preferably a trialkylamine having 5 to 10 carbon atoms.

The component (D2) may be used alone or in a combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (Al).

In a case where the content of the component (D2) is greater than or equal to the lower limits of the above-described preferable ranges, excellent lithography characteristics and an excellent resist pattern shape are easily obtained. On the other hand, in a case where the content is less than or equal to the upper limits of the above-described ranges, the sensitivity can be satisfactorily maintained and the throughput is also excellent.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in a combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

<<Fluorine additive component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

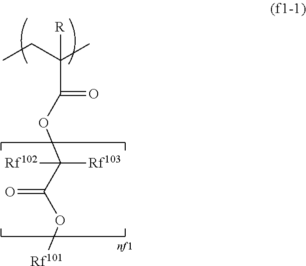

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represent a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory enough solubility in a solvent for a resist to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in a combination of two or more kinds thereof. In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition of the present embodiment can be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those known as solvents of a chemically amplified resist composition and then used.

Examples of the component (S) include lactones such as y-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as a monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment as appropriate.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the compound (B0) (the component (B0)) represented by General Formula (b0). The anion moiety of the component (B0) has a specific bulky structure (fused cyclic group in which an aromatic ring and an alicyclic ring are fused). In this manner, the diffusion length of an acid generated from the component (B0) upon light exposure can be suitably controlled. Further, since the anion moiety of the component (B0) has a bromine atom or an iodine atom, the uniformity of the component (B0) in the resist film is increased due to the improvement of hydrophobicity.

In addition, the anion moiety of the component (B0) contains a hydrocarbon group having a bromine atom or a hydrocarbon group having an iodine atom. Since a bromine atom and an iodine atom have high absorption efficiency of extreme ultraviolet rays (EUV) and electron beams (EB), the sensitivity to EUV and EB can be further improved as compared with an acid generator that does not have a bromine atom or an iodine atom in the related art.

Therefore, it is assumed that the resist composition containing the component (B0) according to the present embodiment is capable of forming a resist pattern with high sensitivity and satisfactory CDU.

### (Resist Pattern Forming Method)

A resist pattern forming method according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern forming method, a resist pattern forming method of performing processes to be described below is an exemplary example.

First, a support is coated with the resist composition of the present embodiment using a spinner or the like, and a bake (post-apply bake (PAB)) treatment is performed under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure bake (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in the case of an alkali developing process and using a developing solution containing an organic solvent (organic developing solution) in the case of a solvent developing process.

After the developing treatment, it is preferable to conduct a rinse treatment. As the rinse treatment, water rinsing using pure water is preferable in the case of the alkali developing process, and rinsing using a rinse solution containing an organic solvent is preferable in the case of the solvent developing process.

In the case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, examples of which include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

Further, as the support, any one of the above-described supports provided with an inorganic and/or organic film on the above-described substrate may be used. As the inorganic film, an inorganic antireflection film (inorganic BARC) can be used. As the organic film, an organic film such as an organic antireflection film (organic BARC) or a lower-layer organic film used in a multilayer resist method can be used.

Here, the multilayer resist method is a method of providing at least one layer of an organic film (lower-layer organic film) and at least one layer of a resist film (upper-layer resist film) on a substrate and performing patterning of the lower-layer organic film using a resist pattern formed on the upper-layer resist film as a mask, and this method is considered to enable formation of a pattern with a high aspect ratio. That is, according to the multilayer resist method, since a desired thickness can be ensured by the lower-layer organic film, the thickness of the resist film can be reduced, and a fine pattern with a high aspect ratio can be formed.

The multilayer resist method is basically classified into a method in which a double-layer structure consisting of an upper-layer resist film and a lower-layer organic film is formed (two-layer resist method), and a method in which a multilayer structure having three or more layers consisting of an upper-layer resist film, a lower-layer organic film, and one or more intermediate layers (thin metal film and the like) provided between the upper-layer resist film and the lower-layer organic film is formed (three-layer resist method).

The wavelength used for light exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays. The resist composition is useful for a KrF excimer laser, an ArF excimer laser, EB, and EUV, more useful for an ArF excimer laser, EB, and EUV, and particularly useful for EB and EUV. That is, the resist pattern forming method according to the present embodiment is a method particularly useful in a case where the step of exposing the resist film to light includes a process of exposing the resist film to extreme ultraviolet (EUV) rays or electron beams (EB).

The method of exposing the resist film to light may be typical exposure (dry exposure) conducted in air or an inert gas such as nitrogen, or liquid immersion exposure (liquid immersion lithography). Among these, liquid immersion exposure is preferable.

The liquid immersion exposure is an exposure method in which the region between the resist film and the lens at the lowermost position of the exposure apparatus is filled with a solvent (liquid immersion medium) in advance that has a refractive index larger than the refractive index of air, and the exposure (immersion exposure) is conducted in this state.

As the liquid immersion medium, a solvent which has a refractive index greater than the refractive index of air but less than the refractive index of the resist film to be exposed to light is preferable. The refractive index of such a solvent is not particularly limited as long as the refractive index is in the above-described range.

Examples of the solvent which has a refractive index that is greater than the refractive index of air but less than the refractive index of the resist film include water, a fluorine-based inert liquid, a silicon-based solvent, and a hydrocarbon-based solvent.

Specific examples of the fluorine-based inert liquid include a liquid containing a fluorine-based compound such as C₃HCl₂F₅, C₄F₉OCH₃, C₄F₉OC₂H₅, or C₅H₃F₇ as a main component, and a liquid with a boiling point of 70° C. to 180° C. is preferable and a liquid with a boiling point of 80° C. to 160° C. is more preferable. A fluorine-based inert liquid having a boiling point in the above-described range is preferable from the viewpoint that a medium used for liquid immersion can be removed using a simple method after completion of light exposure.

As the fluorine-based inert liquid, a perfluoroalkyl compound in which all hydrogen atoms in the alkyl group have been substituted with fluorine atoms is particularly preferable. Specific examples of the perfluoroalkyl compound include a perfluoroalkylether compound and a perfluoroalkylamine compound.

Further, specific examples of the perfluoroalkylether compound include perfluoro(2-butyl-tetrahydrofuran)(boiling point of 102° C.), and specific examples of the perfluoroalkylamine compound include perfluorotributylamine (boiling point of 174° C.).

As the liquid immersion medium, water is preferable from the viewpoints of cost, safety, environmental issues, and versatility.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

Some organic solvents have a plurality of the functional groups which characterize each of the solvents in the structure thereof. In such a case, the organic solvents are considered to correspond to all the solvents containing the functional groups. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. Among these, a fluorine atom is preferable as the halogen atom.

Among the examples, as the organic solvent contained in the organic developing solution, a polar solvent is preferable. Further, a ketone-based solvent, an ester-based solvent, and a nitrile-based solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these examples, methyl amyl ketone (2-heptanone) is preferable as the ketone-based solvent.

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl-2-hydroxypropionate, ethyl-2-hydroxypropionate, methyl-3-methoxypropionate, ethyl-3-methoxypropionate, ethyl-3-ethoxypropionate, and propyl-3-methoxypropionate. Among these examples, butyl acetate is preferable as the ester-based solvent.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution onto the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in a combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution onto the surface of a support (spray method).

According to the resist pattern forming method of the present embodiment described above, since the resist composition described above is used, a resist pattern with high sensitivity and satisfactory CDU can be formed.

It is preferable that various materials that are used in the resist composition according to the above-described embodiment and the pattern forming method according to the above-described embodiment (for example, a resist solvent, a developing solution, a rinse solution, a composition for forming an antireflection film, and a composition for forming a top coat) not contain impurities such as a metal, a metal salt containing a halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, and it is most preferable that the impurities be substantially absent (less than or equal to the detection limit of the measuring device).

### (Compound)

The compound according to the third aspect of the present invention is a compound represented by General Formula (b0).

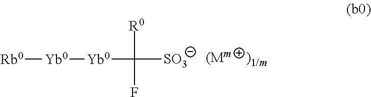

[In the formula, Rb⁰ represents a fused cyclic group in which an aromatic ring and an alicyclic ring are fused. The alicyclic ring in the fused cyclic group has substituents, and at least one of the substituents contains a hydrocarbon group having an iodine atom. Yb⁰ represents a divalent linking group or a single bond. Here, Yb⁰ is bonded to the alicyclic ring in the fused cyclic group. Vb⁰ represents a single bond, an alkylene group, or a fluorinated alkylene group. R⁰ represents a fluorinated alkyl group having 1 to 5 carbon atoms or a fluorine atom. M^{m+} represents an m-valent organic cation. m represents an integer of 1 or greater.]

The compound represented by General Formula (b0) is the same as the component (B0) in the resist composition according to the first aspect of the present invention described above.

[Method of Producing Compound Represented by General Formula (b0)]

The component (B0) can be produced by using a known method.

As a specific method of producing the component (B0), a method of producing a compound represented by General Formula (b′0), which is an example of the component (B0), is described below.

First, a compound X1 represented by General Formula (X-1) reacts with a compound Alc1 represented by General Formula (Alc-1) which contains a desired hydrocarbon group having a bromine atom or a hydrocarbon group (Rbi) having an iodine atom, to obtain a compound X2 represented by General Formula (X-2) (first step).

Next, the compound X2 reacts with a compound I1 represented by General Formula (I-1) having a desired anion group to obtain a precursor Bpre represented by General Formula (Bpre) (second step).

Next, the precursor Bpre and a compound S1 represented by General Formula (S-1) are subjected to a salt exchange reaction, which makes it possible to obtain a compound represented by General Formula (b′0), which is an example of the component (B0) (third step).

Further, in the following reaction formula, “RbiO—C═O—Rb⁰⁰” which is noted for convenience is an example of “Rb⁰” in General Formula (b0).

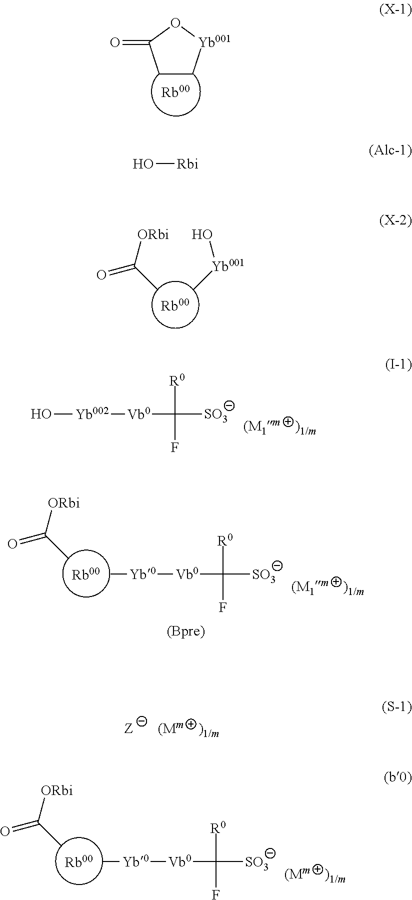

[In the formulae, Rb⁰⁰ represents a fused cyclic group in which an aromatic ring and an alicyclic ring are fused. Yb⁰⁰¹ represents a single bond or a divalent linking group. Rbi represents a hydrocarbon group having a bromine atom or a hydrocarbon group having an iodine atom. Yb⁰⁰² represents a single bond or a divalent linking group. Vb⁰ represents a single bond, an alkylene group, or a fluorinated alkylene group. R⁰represents a fluorinated alkyl group having 1 to 5 carbon atoms or a fluorine atom. (M₁″^{m+})_{1/m} represents an ammonium cation. Yb′⁰ represents a divalent linking group. Z⁻ represents a non-nucleophilic ion. (Mm⁺)_{1/m} represents an m-valent organic cation. m represents an integer of 1 or greater.]

### First Step:

The first step is, for example, a step of dissolving the compound X1 and the compound Alc1 in an organic solvent (THF or the like) and reacting the mixture in the presence of a base to obtain a compound X2.

Specific examples of the base include sodium hydride, K₂CO₃, Cs₂CO₃, lithium diisopropylamide (LDA), triethylamine, and 4-dimethylaminopyridine.

The reaction temperature is, for example, in a range of 0° C. to 50° C., and the reaction time is, for example, 10 minutes or longer and 24 hours or shorter.

In the formula, Rb⁰⁰ represents a fused cyclic group in which an aromatic ring and an alicyclic ring are fused, and the fused cyclic group has the same definition as the fused cyclic group in which an aromatic ring and an alicyclic ring are fused, as Rb⁰ in General Formula (b0).

In the formula, Yb⁰⁰¹ represents a single bond or a divalent linking group, and examples of the divalent linking group include —CO—, —NH—, an alkylene group having —CO—, and an alkylene group having —NH—.

### Second Step:

The second step is, for example, a step of dissolving the compound X2 and the compound 11 in an organic solvent (dichloromethane or the like) and subjecting them to a condensation reaction in the presence of a base to obtain the precursor Bpre.

Specific examples of the base include organic bases such as triethylamine, 4-dimethylaminopyridine, pyridine, ethyldiisopropylaminocarbodiimide (EDCI) hydrochloride, dicyclohexylcarboxyimide (DCC), N,N-diisopropylcarbodiimide, and carbodiimidazole, and inorganic bases such as sodium hydride, K₂CO₃, Cs₂CO₃.

In the formulae, Vb⁰ and R⁰ have the same definitions as Vb⁰ and R⁰ in General Formula (b0), respectively.

In the formulae, Yb⁰⁰² represents a single bond or a divalent linking group, and examples of the divalent linking group include —CO—, —NH—, an alkylene group having —CO—, and an alkylene group having —NH—.

In the formulae, Yb′⁰ represents a divalent linking group, and specifically, the divalent linking group is a group formed by reacting —Yb⁰⁰¹-OH of the compound X2 with —Yb⁰⁰²—OH of the compound I1 and specifically, an —Yb⁰⁰¹—O—Yb⁰⁰²— group.

For example, the compound X2 is a carboxylic acid, and the compound I1 is an alcohol.

In the formulae, (M₁″^{m+})_{1/m} represents an ammonium cation, and the ammonium cation may be an ammonium cation derived from an aliphatic amine or an ammonium cation derived from an aromatic amine.

The used amount of the compound I1 is, for example, in a range of 0.5 to 3 equivalents with respect to the compound X2.

The reaction temperature is, for example, in a range of 0° C. to 50° C., and the reaction time is, for example, 10 minutes or longer and 24 hours or shorter.

### Third Step:

The third step is, for example, a step of reacting the precursor Bpre with the compound S1 for salt exchange in a solvent such as water, dichloromethane, acetonitrile, or chloroform and exchanging the cation of the precursor Bpre with the cation of the compound S1 to obtain a compound represented by General Formula (b′0), which is an example of the component (B0).

In the formulae, examples of Z⁻ include ions that can be an acid having an acidity lower than that of the precursor Bpre, and specific examples thereof include a halogen ion such as a bromine ion or a chloride ion, BF₄⁻, AsF₆⁻, SbF₆⁻, PF₆⁻, and ClO₄⁻.

The reaction temperature is, for example, in a range of 0° C. to 100° C., and the reaction time is, for example, 10 minutes or longer and 24 hours or shorter.

In the formulae, (M^{m+})_{1/m} has the same definition as (M^{m+})_{1/m} in General Formula (b0).

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

As the raw material that is used in each step, a commercially available raw material may be used, or a synthetic material may be used.

For example, in the case of synthesizing the compound X1, the compound X1 can be obtained by carrying out a Diels-Alder reaction between an aromatic compound (for example, anthracene) and an alkene (for example, maleic acid anhydride).

The compound according to the third aspect of the present invention described above is a compound useful as an acid generator in the resist composition according to the first aspect of the present invention described above.

### (Acid Generator)

The acid generator according to the fourth aspect of the present invention contains the compound according to the third aspect described above.

Such an acid generator is useful as an acid generator component for a chemically amplified resist composition. In a case where such an acid generator component is used in a chemically amplified resist composition, the sensitivity is increased and CDU is further improved in the resist pattern formation. In a case where such an acid generator component is used, the sensitivity is increased and CDU is further improved particularly in the resist pattern formation carried out using a light source such as EB or EUV.

### EXAMPLES

Hereinafter, the present invention will be described in more detail based on examples, but the present invention is not limited to these examples.

### <Production of Compound X1>

### Production Example 1

Anthracene (20.0 g, 112.2 mmol), maleic acid anhydride (16.6 g, 168.3 mmol), aluminum chloride (1.50 g, 11.2 mmol), and toluene (200 g) were placed in a 300 mL three-neck flask and reacted at 80° C. for 4 hours with stirring. After cooling, ultrapure water (155 g) was added thereto, and after carrying out stirring for 30 minutes, the precipitated solid was filtered. The filtrate was dissolved in a mixed solvent of THF (93 g) and dichloromethane (680 g) and washed 3 times with ultrapure water (155 g), and the organic layer was concentrated using a rotary evaporator. The concentrate was recrystallized with ethyl acetate to obtain a compound (X-1-1).

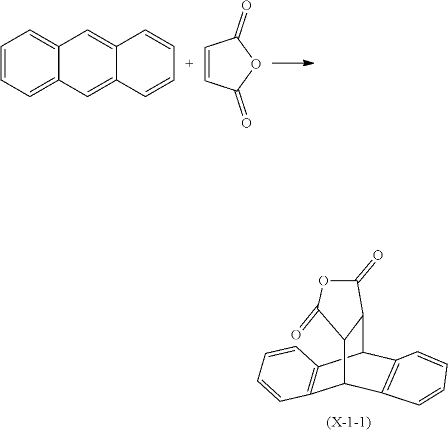

### Production of compound X2

### Production Example 2-1

Sodium hydride (60%, in oil) (4.3 g, 106.0 mmol) and dewatered THF (73.2 g) were put into a 300 mL three-neck flask and cooled to 10° C. or lower. 2,4,6-Triiodophenol (25.0 g, 53.0 mmol) was added to the suspension, the mixture was stirred for 30 minutes, a compound (X-1-1) (14.6 g, 53.0 mmol) was added thereto, and the temperature was returned to room temperature (25° C.). After 6 hours, the reaction solution was added dropwise to 5% hydrochloric acid (91.1 g, 127.2 mmol) cooled to 10° C. or lower, the solution was stirred for 1 hour and subjected to liquid separation, and the organic layer was concentrated using a rotary evaporator. Dichloromethane (79 g) was added to the concentrate, the mixture was stirred at room temperature (25° C.) for 2 hours, and the precipitated solid was filtered. Acetonitrile (337 g) was added to the obtained solid to dissolve the solid at 60° C., the temperature was returned to room temperature (25° C.), ultrapure water (337 g) was added thereto, and the solution was cooled to 10° C. or lower. After 2 hours, the precipitated solid was filtered, thereby obtaining a compound (X-2-1).

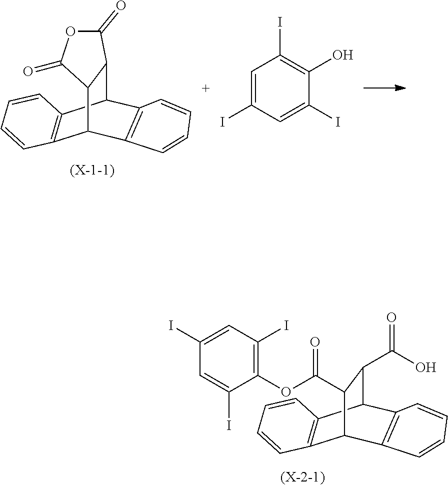

### Production Example 2-2

A compound (X-2-2) was obtained in the same manner as in the production example of the compound (X-2-1) except that 2,4,6-triiodophenol (25.0 g, 53.0 mmol) was changed to 2,4-diiodophenol (18.3 g, 52.9 mmol).

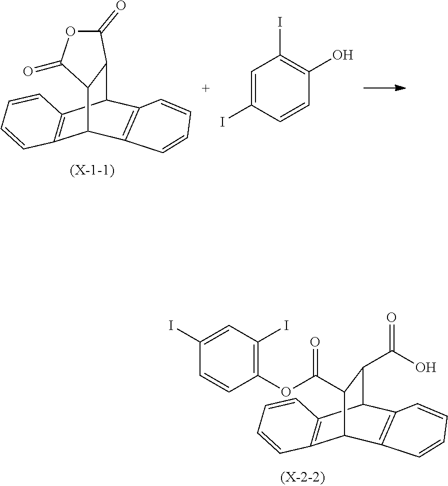

### Production Example 2-3

A compound (X-2-3) was obtained in the same manner as in the production example of the compound (X-2-1) except that 2,4,6-triiodophenol (25.0 g, 53.0 mmol) was changed to 4-iodophenol (11.7 g, 53.2 mmol).

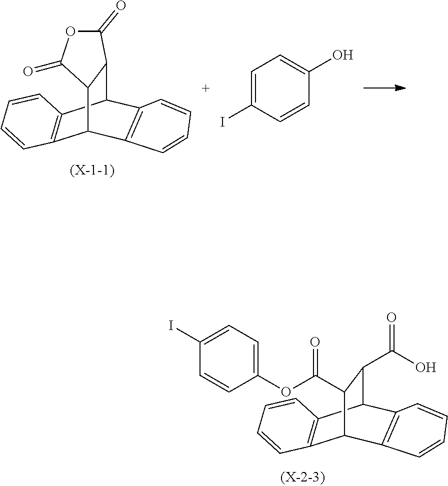

### Production Example 2-4

A compound (X-2-4) was obtained in the same manner as in the production example of the compound (X-2-1) except that 2,4,6-triiodophenol (25.0 g, 53.0 mmol) was changed to 2-fluoro-4-iodophenol (12.6 g, 52.9 mmol).

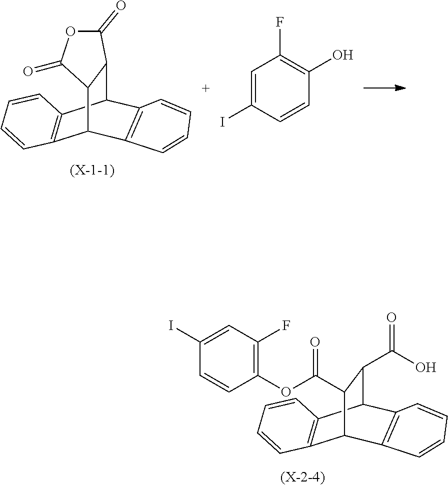

### <Production of Precursor Bpre>

### Production Example 3-1

The compound (X-2-1) (15.0 g, 20.1 mmol), the compound (T-1-1) (6.9 g, 22.1 mmol), and dichloromethane (200 g) were put into a 500 mL three-neck flask and stirred at room temperature (25° C.) for 10 minutes. Next, N,N′-diisopropylcarbodiimide (2.8 g, 22.1 mmol) and dimethylaminopyridine (0.031 g, 0.3 mmol) were added thereto and the mixture was allowed to react at room temperature (25° C.) for 12 hours. The reaction solution was washed with ultrapure water (100 g) four times, and the organic layer was concentrated using a rotary evaporator. Ethyl acetate (100 g) was added to the concentrate, the mixture was stirred at room temperature (25° C.) for 2 hours, and the precipitated solid was filtered. Methanol (50 g) was added to the obtained solid to dissolve the solid at 60° C., and the solution was concentrated using a rotary evaporator. Ethyl acetate (100 g) was added to the concentrate, the solution was stirred at room temperature for 2 hours, and the precipitated solid was filtered. This operation was repeated two times, thereby obtaining a precursor (Bpre-01).

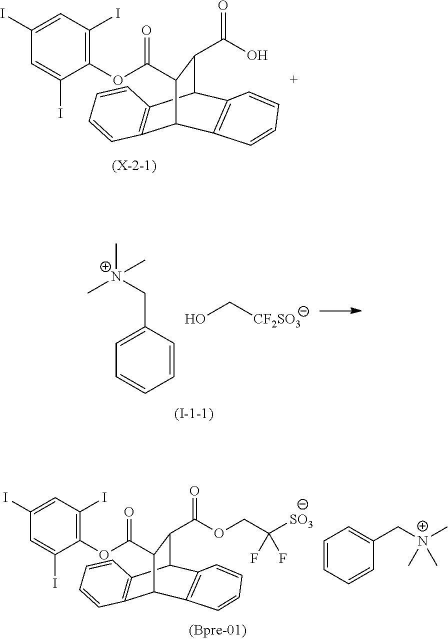

### Production Example 3-2

A precursor (Bpre-02) was obtained by the same method as the precursor (Bpre-01) except that the compound (I-1-1) (6.9 g, 22.1 mmol) was changed to the compound (I-1-2) (8.4 g, 22.1 mmol).

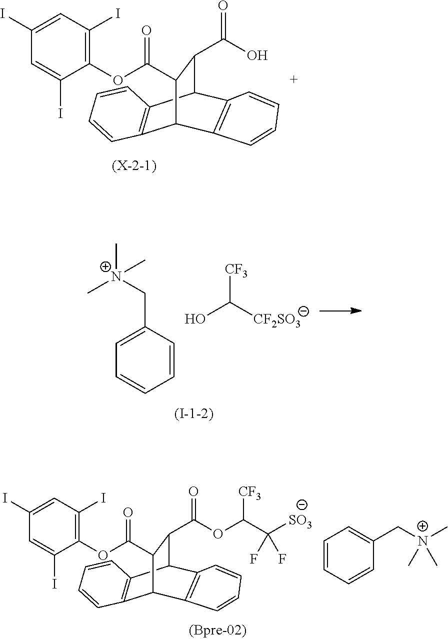

### Production Example 3-3

A precursor (Bpre-03) was obtained by the same method as the precursor (Bpre-01) except that the compound (X-2-1) (15.0 g, 20.1 mmol) was changed to the compound (X-2-2) (12.5 g, 20.1 mmol).

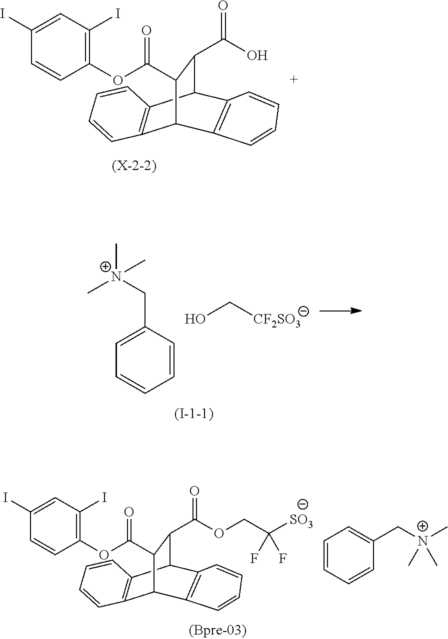

### Production Example 3-4

A precursor (Bpre-04) was obtained by the same method as the precursor (Bpre-01) except that the compound (X-2-1) (15.0 g, 20.1 mmol) was changed to the compound (X-2-3) (10.0 g, 20.1 mmol).

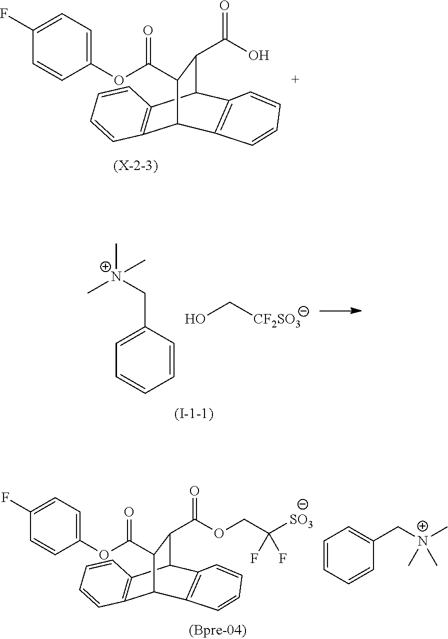

### Production Example 3-5

A precursor (Bpre-05) was obtained by the same method as the precursor (Bpre-01) except that the compound (X-2-1) (15.0 g, 20.1 mmol) was changed to the compound (X-2-4) (10.3 g, 20.0 mmol).

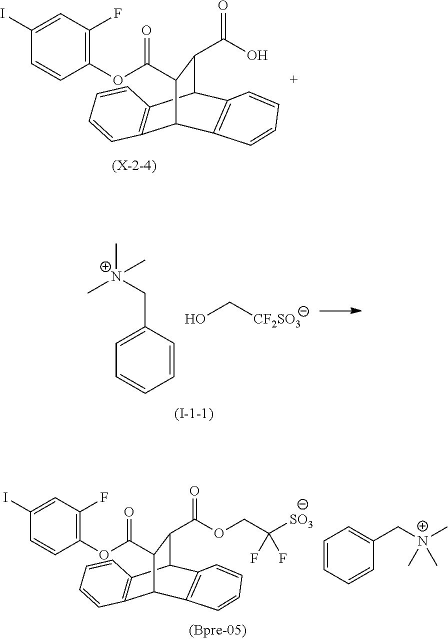

### Production Example of Compound (B0-01)

The precursor (Bpre-01) (15.0 g, 14.4 mmol) and a salt exchange compound (S-1-1) (4.94 g, 14.4 mmol) were dissolved in dichloromethane (170 g) and ultrapure water (170 g), and the solution was allowed to react at room temperature (25° C.) for 30 minutes. After completion of the reaction, the water layer was removed, and the organic layer was washed four times with ultrapure water (85 g). The organic layer was concentrated and dried using a rotary evaporator, thereby obtaining a compound (B0-01).

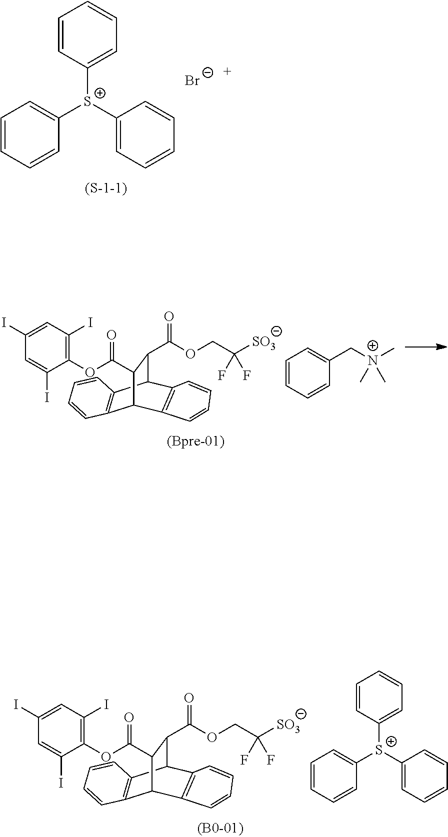

Compounds (B0-02) to (B0-09) shown below were obtained in the same manner as in the section of “Production example of compound (B0-01)” except that the combination of the precursor (Bpre-01) in the section of “Production example of compound (B0-O1)” described above with the salt exchange compound (S-1-1) was changed to each of the above-described precursors (Bpre-01) to (Bpre-05) and each of the following salt exchange compounds (S-1-1) to (S-1-4).

The structures of the compound (B0-01) to the compound (B0-09) are shown below.

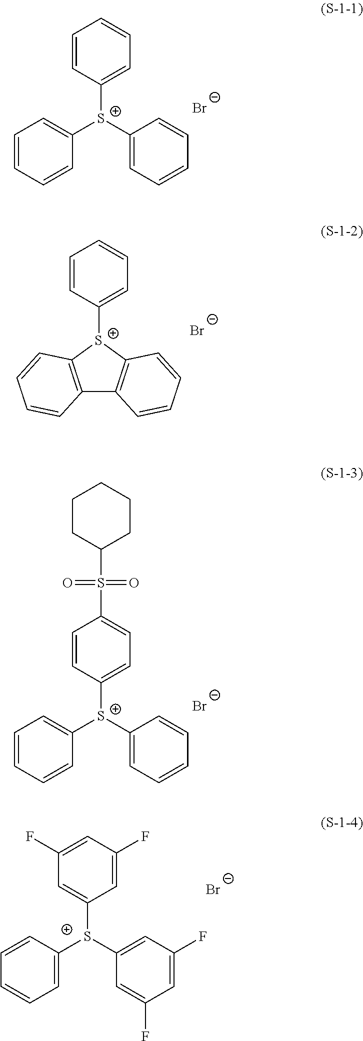

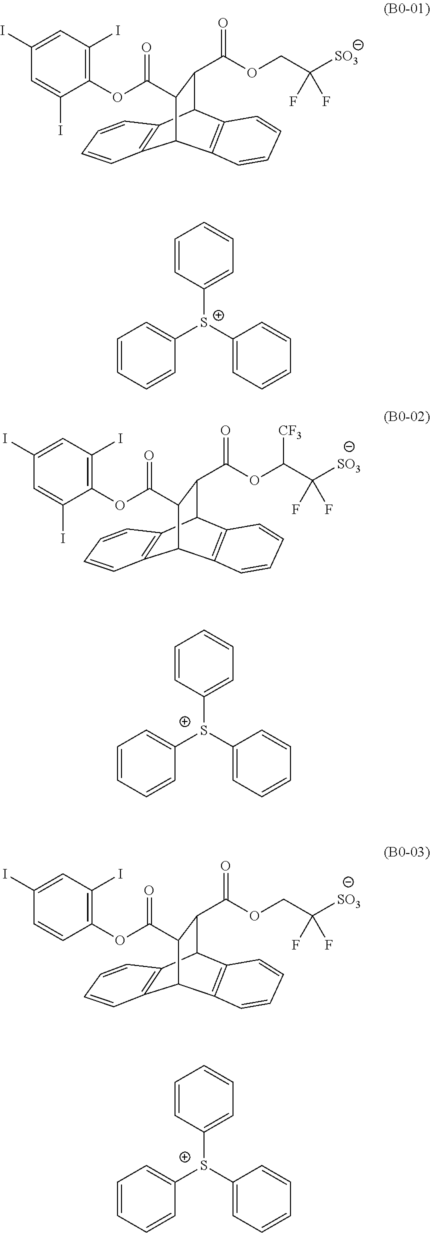

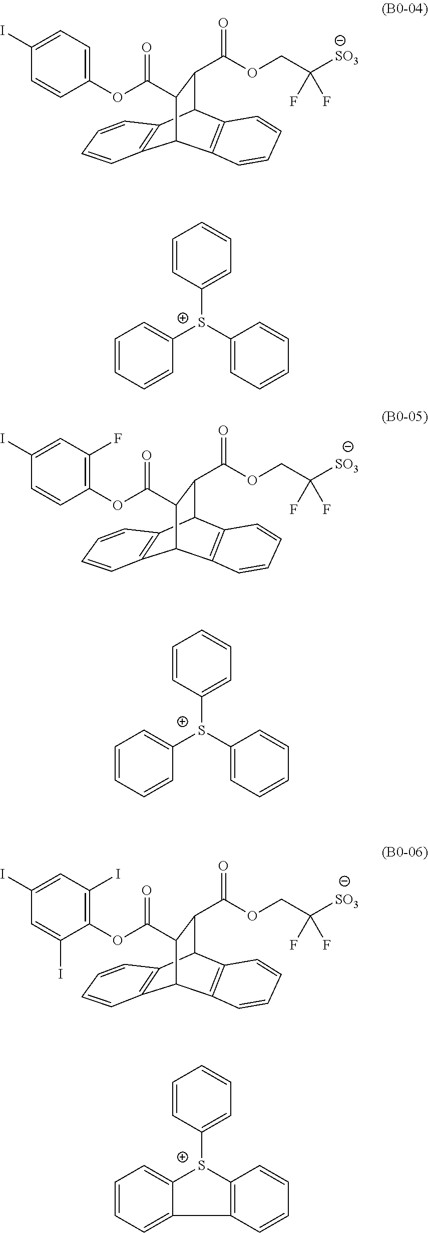

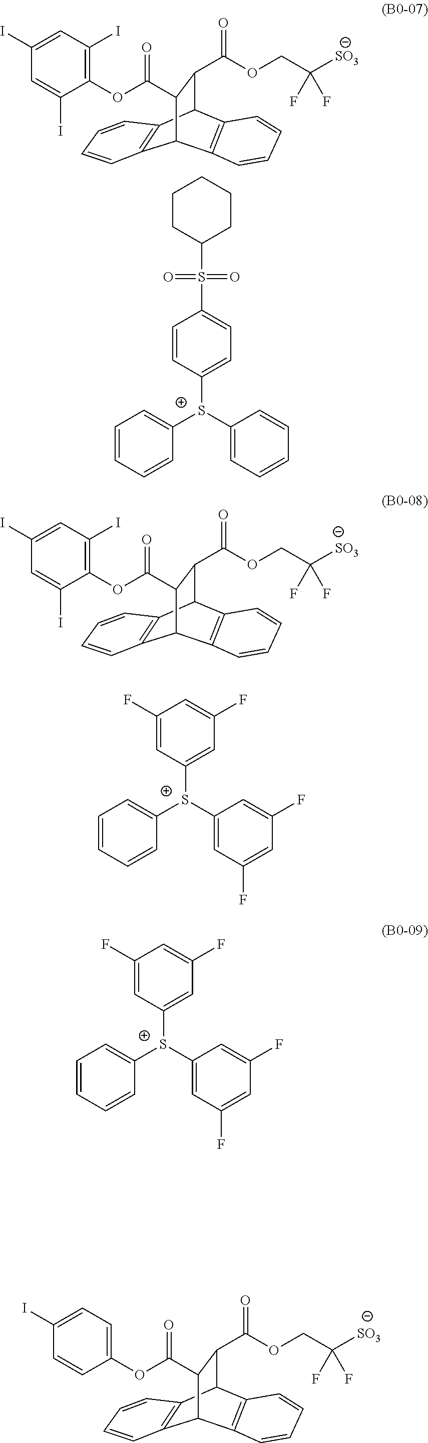

Further, the structures of the above-described compounds (B0-01) to (B0-09) were identified from the analysis results of the ¹H-NMR measurement shown below.

Compound (B0-01): combination of precursor (Bpre-01) and salt exchange compound (S-1-1)

¹H-NMR (DMSO, 400 MHz): δ (ppm)=8.00 (d, I-ArH, 2H), 7.90-7.74 (m, ArH, 15H), 7.50-7.44 (m, ArH, 3H), 7.31-7.29 (m, ArH, 1H), 7.18-7.12 (m, ArH, 4H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H)

Compound (B0-02): combination of precursor (Bpre-02) and salt exchange compound (S-1-1)

¹H-NMR (DMSO, 400 MHz): δ (ppm)=8.00 (d, I-ArH, 2H), 7.90-7.74 (m, ArH, 15H), 7.50-7.44 (m, ArH, 3H), 7.31-7.29 (m, ArH, 1H), 7.18-7.12 (m, ArH, 4H), 5.90 (m, —CF₃CHCF₂—), 5.02 (d, CH, 1H), 4.84 (d, CH, 1H), 3.58-3.57 (m, —OCO—CH—CH—COO, 1H), 3.42-3.40 (m, —OCO—CH—CH—COO, 1H)

Compound (B0-03): combination of precursor (Bpre-03) and salt exchange compound (S-1-1)

¹H-NMR (DMSO, 400 MHz): δ (ppm)=7.99 (d, I-ArH, 1H), 7.90-7.74 (m, ArH, I-ArH, 16H), 7.50-7.44 (m, ArH, 3H), 7.31-7.29 (m, ArH, 1H), 7.18-7.12 (m, ArH, 4H), 6.90 (dd, I-ArH, 1H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H)

Compound (B0-04): combination of precursor (Bpre-04) and salt exchange compound (S-1-1)

¹H-NMR (DMSO, 400 MHz): δ (ppm)=7.90-7.74 (m, ArH, I-ArH, 17H), 7.50-7.44 (m, ArH, 3H), 7.31-7.29 (m, ArH, 1H), 7.18-7.12 (m, ArH, 4H), 6.89 (dd, I-ArH, 2H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (in, —OCO—CH—CH—COO, 1H)

Compound (B0-05): combination of precursor (Bpre-05) and salt exchange compound (S-1-1)

¹H-NMR (DMSO, 400 MHz): δ (ppm)=7.90-7.74 (m, ArH, 15H), 7.50-7.40 (m, ArH, I-ArH, 5H), 7.31-7.29 (m, ArH, 1H), 7.18-7.12 (m, ArH, 4H), 7.02-7.00 (m, I-ArH, 1H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H)

Compound (B0-06): combination of precursor (Bpre-01) and salt exchange compound (S-1-2)

¹H-NMR (DMSO, 400 MHz): δ (ppm)=8.50 (d, ArH, 2H), 8.37 (d, ArH, 2H), 8.00 (d, 1-ArH, 2H), 7.93 (t, ArH, 2H), 7.75-7.55 (m, Ar, 7H),

7.50-7.44 (m, ArH, 3H), 7.31-7.29 (m, ArH, I H), 7.18-7.12 (m, ArH, 4H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, I H)

Compound (B0-07): combination of precursor (Bpre-01) and salt exchange compound (S-1-3)

¹H-NMR (DMSO, 400 MHz): δ (ppm)=8.22-7.70 (m, ArH, I-ArH, 16H), 7.50-7.44 (m, ArH, 3H), 7.31-7.29 (m, ArH, 1H), 7.18-7.12 (m, ArH, 4H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H), 2.77 (m, cyclohexyl, 1H), 2.11-1.12 (m, chclohexyl, 10H)

Compound (B0-08): combination of precursor (Bpre-01) and salt exchange compound (S-1-4)

¹H-NMR (DMSO, 400 MHz): δ (ppm)=8.00 (d, I-ArH, 2H), 7.98-7.77 (m, ArH, 11H), 7.50-7.44 (m, ArH, 3H), 7.31-7.29 (m, ArH, 1H), 7.18-7.12 (m, ArH, 4H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H)

Compound (B0-09): combination of precursor (Bpre-04) and salt exchange compound (S-1-4)

¹H-NMR (DMSO, 400 MHz): δ (ppm)=7.98-7.77 (m, ArH, I-ArH, 13H), 7.50-7.44 (m, ArH, 3H), 7.31-7.29 (m, ArH, 1H), 7.18-7.12 (m, ArH, 4H), 6.89 (dd, I-ArH, 2H), 5.02 (d, CH, 1H), 4.84 (d, CH, 1H), 4.66-4.38 (m, —CH₂CF₂—, 2H), 3.58-3.57 (m, —OCO—CH—CH—COO, 1H), 3.42-3.40 (m, —OCO—CH—CH—COO, 1H)

### <Preparation of Resist Composition>

### Examples 1 to 18 and Comparative Examples 1 to 4

Each of the components listed in Tables 1 to 3 was mixed and dissolved to prepare a resist composition of each example.

TABLE 1

| | | Component | | |
|---|---|---|---|---|
| | | (B) | | |
| | Component | Component | Component | Component |
| | (A) | (B0) | (D) | (S) |
| Example 1 | (A)-1 | (B0)-1 | (D)-1 | (S)-1 |
| | [100] | [35.2] | [5] | [8000] |
| Example 2 | (A)-1 | (B0)-2 | (D)-1 | (S)-1 |
| | [100] | [37.2] | [5] | [8000] |
| Example 3 | (A)-1 | (B0)-3 | (D)-1 | (S)-1 |
| | [100] | [31.3] | [5] | [8000] |
| Example 4 | (A)-1 | (B0)-4 | (D)-1 | (S)-1 |
| | [100] | [27.5] | [5] | [8000] |
| Example 5 | (A)-1 | (B0)-5 | (D)-1 | (S)-1 |
| | [100] | [28.0] | [5] | [8000] |
| Example 6 | (A)-1 | (B0)-6 | (D)-1 | (S)-1 |
| | [100] | [35.1] | [5] | [8000] |
| Example 7 | (A)-1 | (B0)-7 | (D)-1 | (S)-1 |
| | [100] | [39.6] | [5] | [8000] |
| Example 8 | (A)-1 | (B0)-8 | (D)-1 | (S)-1 |
| | [100] | [37.4] | [5] | [8000] |
| Example 9 | (A)-2 | (B0)-1 | (D)-1 | (S)-1 |
| | [100] | [35.2] | [5] | [8000] |

TABLE 2

| | | Component | | |
|---|---|---|---|---|
| | | (B) | | |
| | Component | Component | Component | Component |
| | (A) | (B1) | (D) | (S) |
| Comparative | (A)-1 | (B1)-1 | (D)-1 | (S)-1 |
| Example 1 | [100] | [20.0] | [5] | [8000] |
| Comparative | (A)-1 | (B1)-2 | (D)-1 | (S)-1 |
| Example 2 | [100] | [22.8] | [5] | [8000] |
| Comparative | (A)-1 | (B1)-3 | (D)-1 | (S)-1 |
| Example 3 | [100] | [34.9] | [5] | [8000] |
| Comparative | (A)-1 | (B1)-4 | (D)-1 | (S)-1 |
| Example 4 | [100] | [35.2] | [5] | [8000] |

TABLE 3

| | | Component | | |
|---|---|---|---|---|
| | | (B) | | |
| | Component | Component | Component | Component |
| | (A) | (B0) | (D) | (S) |
| Example 10 | (A)-1 | (B0)-9 | (D)-1 | (S)-1 |
| | [100] | [29.6] | [5] | [8000] |
| Example 11 | (A)-3 | (B0)-9 | (D)-1 | (S)-1 |
| | [100] | [29.6] | [5] | [8000] |
| Example 12 | (A)-4 | (B0)-9 | (D)-1 | (S)-1 |
| | [100] | [29.6] | [5] | [8000] |
| Example 13 | (A)-5 | (B0)-9 | (D)-1 | (S)-1 |
| | [100] | [29.6] | [5] | [8000] |
| Example 14 | (A)-6 | (B0)-9 | (D)-1 | (S)-1 |
| | [100] | [29.6] | [5] | [8000] |
| Example 15 | (A)-3 | (B0)-8 | (D)-1 | (S)-1 |
| | [100] | [37.4] | [5] | [8000] |
| Example 16 | (A)-4 | (B0)-8 | (D)-1 | (S)-1 |
| | [100] | [37.4] | [5] | [8000] |
| Example 17 | (A)-5 | (B0)-8 | (D)-1 | (S)-1 |
| | [100] | [37.4] | [5] | [8000] |
| Example 18 | (A)-6 | (B0)-8 | (D)-1 | (S)-1 |
| | [100] | [37.4] | [5] | [8000] |

The abbreviations in Tables 1 to 3 have the following meanings. The numerical values in the brackets are blending amounts (parts by mass).
(A)-1: polymer compound represented by Chemical Formula (A1)-1. The weight-average molecular weight (Mw) of the polymer compound (A1)-i in terms of standard polystyrene determined by GPC measurement was 7100, and the molecular weight dispersity (Mw/Mn) thereof was 1.69. The copolymerization compositional ratio (the ratio (molar ratio) of each constitutional unit in the structural formula) determined by ¹³C-NMR was 1/m=50/50.
(A)-2: polymer compound represented by Chemical Formula (Al)-2. The weight-average molecular weight (Mw) of the polymer compound (A1)-2 in terms of standard polystyrene determined by GPC measurement was 7000, and the molecular weight dispersity (Mw/Mn) thereof was 1.72. The copolymerization compositional ratio (the ratio (molar ratio) of each constitutional unit in the structural formula) determined by ¹³C-NMR was l/m=50/50.
(A)-3: polymer compound represented by Chemical Formula (A1)-3. The weight-average molecular weight (Mw) of the polymer compound (A1)-3 in terms of standard polystyrene determined by GPC measurement was 6900, and the molecular weight dispersity (Mw/Mn) thereof was 1.68. The copolymerization compositional ratio (the ratio (molar ratio) of each constitutional unit in the structural formula) determined by ¹³C-NMR was I/m=50/50.
(A)-4: polymer compound represented by Chemical Formula (A1)-4. The weight-average molecular weight (Mw) of the polymer compound (Al)-4 in terms of standard polystyrene determined by GPC measurement was 7000, and the molecular weight dispersity (Mw/Mn) thereof was 1.70. The copolymerization compositional ratio (the ratio (molar ratio) of each constitutional unit in the structural formula) determined by ¹³C-NMR was I/m=50/50.
(A)-5: polymer compound represented by Chemical Formula (A1)-5. The weight-average molecular weight (Mw) of the polymer compound (A1)-5 in terms of standard polystyrene determined by GPC measurement was 6800, and the molecular weight dispersity (Mw/Mn) thereof was 1.68. The copolymerization compositional ratio (the ratio (molar ratio) of each constitutional unit in the structural formula) determined by ¹³C-NMR was 1/m=50/50.
(A)-6: polymer compound represented by Chemical Formula (Al)-6. The weight-average molecular weight (Mw) of the polymer compound (A l)-6 in terms of standard polystyrene determined by GPC measurement was 6800, and the molecular weight dispersity (Mw/Mn) thereof was 1.69. The copolymerization compositional ratio (the ratio (molar ratio) of each constitutional unit in the structural formula) determined by ¹³C-NMR was 1/m=50/50.

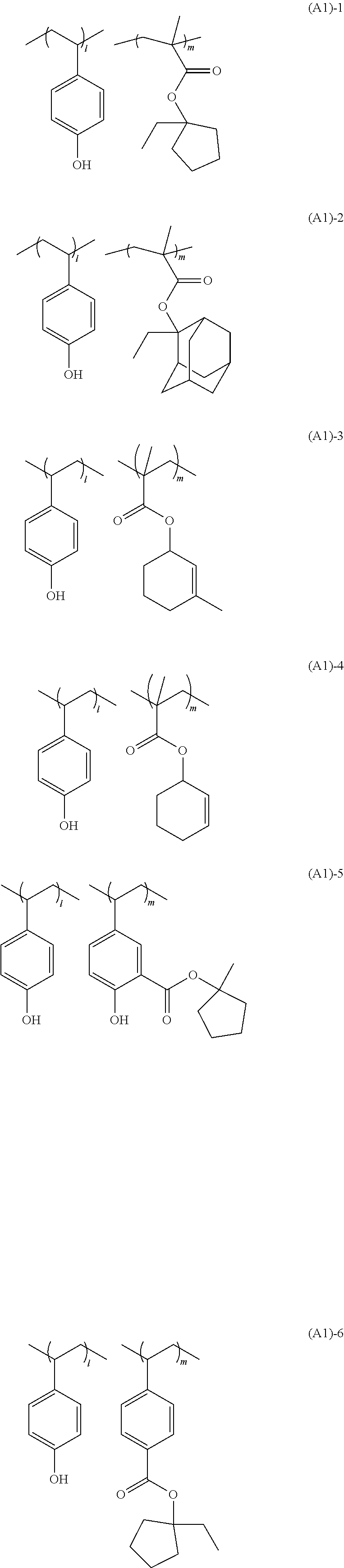

(B0)-1 to (B0)-8: acid generators each consisting of compounds (B0-O1) to (B0-08) shown above
(B1)-1: acid generator consisting of compound (B1-1) shown below
(B1)-2: acid generator consisting of compound (B1-2) shown below
(B1)-3: acid generator consisting of compound (B1-3) shown below
(B1)-4: acid generator consisting of compound (B1-4) shown below

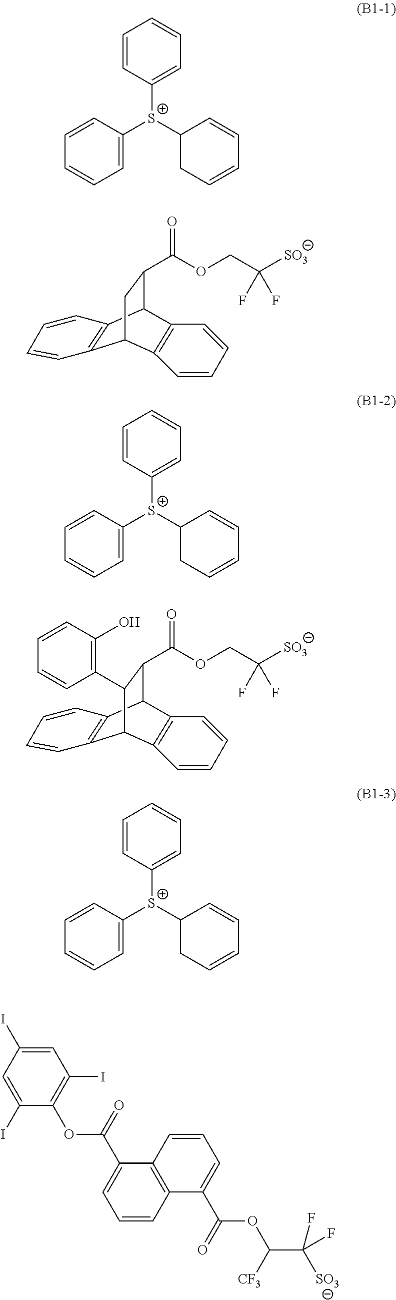

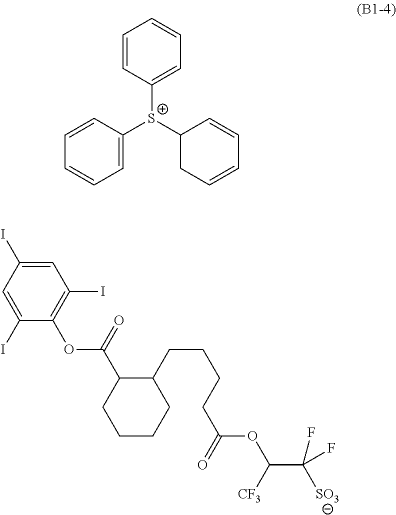

(D)-1: acid diffusion control agent consisting of compound represented by Chemical Formula (D1-1)
(S)-1: mixed solvent of propylene glycol monomethyl ether acetate/propylene glycol monomethyl ether at mass ratio of 60/40

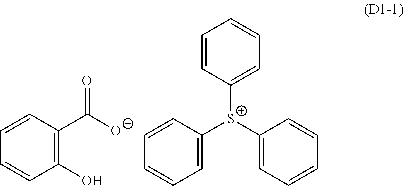 [SMILES: O=C([O-])c1ccccc1O ; c1ccc([S+](c2ccccc2)c2ccccc2)cc1]

### <Resist Pattern Formation>

The resist composition of each Example was applied onto an 8-inch silicon substrate which had been subjected to a hexamethyldisilazane (HMDS) treatment using a spinner, and the coated wafer was subjected to a pre-baking (PAB) treatment on a hot plate at a temperature of 1I 0° C. for 60 seconds so that the coated wafer was dried to form a resist film having a film thickness of 50 nm.

Next, the resist film was subjected to drawing (exposure) to obtain a contact hole pattern (hereinafter, referred to as “CH pattern”) in which holes having a diameter of 32 nm were arranged at equal intervals (pitch: 64 nm) by using an electron beam lithography apparatus JEOL-JBX-9300FS (manufactured by JEOL Ltd.) at an acceleration voltage of 100 kV. Thereafter, a post-exposure baking (PEB) treatment was carried out on the resist film at 1I 0° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.).

Thereafter, water rinsing was carried out with pure water for 15 seconds.

As a result of the above, a CH pattern in which holes having a diameter of 32 nm were arranged at equal intervals (pitch: 64 nm) was formed.

### [Evaluation of Optimum Exposure Amount (Eop)]

According to <Resist pattern formation>described above, an optimum exposure amount Eop (μC/cm²) in which a CH pattern having the target size was formed was determined. The results are listed in Tables 4 and 5 in the columns of “Eop (C/cm²)”.

### [Evaluation of Critical Dimension Uniformity (CDU) of Pattern Dimensions]

The CH pattern formed according to <Resist pattern formation>described above was observed from the upper side of the CH pattern, and the hole diameter (nm) of each of the holes was measured with a scanning electron microscope (CD-SEM, acceleration voltage: 500 V, product name: CG5000, manufactured by Hitachi High-Tech Corporation). Then, the triple value (3σ) of the standard deviation (σ) calculated from the measurement result was determined. The results are listed in Tables 4 and 5 in the columns of “CDU (nm)”.

The lower the value of 3σ determined as described above is, the higher the critical dimension (CD) uniformity of the plurality of holes formed in the resist film is.

TABLE 4

| | PAB | PEB | Eop | CDU |
|---|---|---|---|---|
| | (° C.) | (° C.) | [μC/cm²] | [nm] |
| Example 1 | 110 | 110 | 85 | 4.0 |
| Example 2 | 110 | 110 | 86 | 4.1 |
| Example 3 | 110 | 110 | 89 | 4.3 |
| Example 4 | 110 | 110 | 92 | 4.5 |
| Example 5 | 110 | 110 | 92 | 4.4 |
| Example 6 | 110 | 110 | 84 | 3.7 |
| Example 7 | 110 | 110 | 84 | 3.8 |
| Example 8 | 110 | 110 | 82 | 3.6 |
| Example 9 | 110 | 110 | 85 | 3.9 |
| Comparative | 110 | 110 | 110 | 5.2 |
| Example 1 | | | | |
| Comparative | 110 | 110 | 112 | 5.6 |
| Example 2 | | | | |
| Comparative | 110 | 110 | 87 | 6.2 |
| Example 3 | | | | |
| Comparative | 110 | 110 | 86 | 5.8 |
| Example 4 | | | | |

TABLE 5

| | PAB | PEB | Eop | CDU |
|---|---|---|---|---|
| | (° C.) | (° C.) | [μC/cm²] | [nm] |
| Example 10 | 110 | 110 | 89 | 4.5 |
| Example 11 | 110 | 110 | 90 | 4.5 |
| Example 12 | 110 | 110 | 90 | 4.6 |
| Example 13 | 110 | 110 | 88 | 4.4 |
| Example 14 | 110 | 110 | 89 | 4.4 |
| Example 15 | 110 | 110 | 83 | 3.6 |
| Example 16 | 110 | 110 | 83 | 3.7 |
| Example 17 | 110 | 110 | 81 | 3.6 |
| Example 18 | 110 | 110 | 82 | 3.6 |

As listed in Tables 4 and 5, it was confirmed that the resist compositions of the examples had higher sensitivity and more satisfactory CDU in the resist pattern formation as compared with the resist compositions of the comparative examples.

The resist compositions of Examples 1, 3, and 4 contain the same component (B0) in the main skeleton, and the numbers of iodine atoms in the anion moiety of the component (B0) are different from each other. The compound (B0-01) contained in the resist composition of Example I has 3 iodine atoms, the compound (B0-03) contained in the resist composition of Example 3 has 2 iodine atoms, and the compound (B0-04) contained in the resist composition of Example 4 has 1 iodine atom.

It was confirmed that since the resist composition of Example 1 has more satisfactory sensitivity and CDU as compared with the resist compositions of Examples 3 and 4, the sensitivity and CDU are improved in a case where the number of iodine atoms in the anion moiety of the component (B0) is increased from 1 to 3.

Further, based on the comparison between the resist composition of Example 4 and the resist composition of Example 5, it was found that there is no difference in sensitivity and CDU depending on the presence or absence of a fluorine atom in the anion moiety of the component (B0).

The resist compositions of Examples 1 and 6 to 8 each contain the component (B0) having the same anion moiety but different cation moieties.

It was found that since the resist compositions of Examples 6 to 8 have more satisfactory sensitivity and CDU as compared with the resist composition of Example 1, the sensitivity and CDU are improved in a case where the decomposability of the cation moiety of the component (B0) is improved.

The resist composition of Comparative Example 3 contains an acid generator consisting of the compound (B1-3) having a polycyclic aromatic hydrocarbon group. The resist composition of Comparative Example 4 contains an acid generator consisting of the compound (B1-4) having a polycyclic aliphatic hydrocarbon group. Since these resist compositions do not contain an acid generator containing a fused cyclic group in which an aromatic ring and an alicyclic ring are fused as in the resist compositions of the examples, the CDU is deteriorated as compared with the resist compositions of the examples.

While preferred embodiments of the present invention have been described and illustrated above, it should be understood that these are exemplary of the present invention and are not to be considered as limiting. Additions, omissions, substitutions, and other modifications can be made without departing from the scope of the invention. Accordingly, the present invention is not to be considered as being limited by the foregoing description and is only limited by the scope of the appended claims.

What is claimed is:

1. A resist composition which generates an acid upon light exposure and whose solubility in a developing solution is changed due to an action of the acid, the resist composition comprising:
a base material component (A) whose solubility in a developing solution is changed due to an action of an acid; and
an acid generator component (B) which generates an acid upon light exposure,
wherein the acid generator component (B) contains a compound (B0) represented by General Formula (b0), 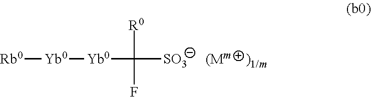
wherein Rb⁰ represents a fused cyclic group in which an aromatic ring and an alicyclic ring are fused, the alicyclic ring in the fused cyclic group has substituents, and at least one of the substituents contains a hydrocarbon group having a bromine atom or a hydrocarbon group having an iodine atom, Yb⁰ represents a divalent linking group or a single bond, where Yb⁰ is bonded to the alicyclic ring in the fused cyclic group, Vb⁰ represents a single bond, an alkylene group, or a fluorinated alkylene group, R⁰ represents a fluorinated alkyl group having 1 to 5 carbon atoms or a fluorine atom, M^{m+} represents an m-valent organic cation, and m represents an integer of 1 or greater.

2. The resist composition according to claim 1, wherein the aromatic ring in Rb⁰ is a benzene ring.

3. The resist composition according to claim 1,
wherein the compound (B0) includes a compound represented by General Formula (b0-1), 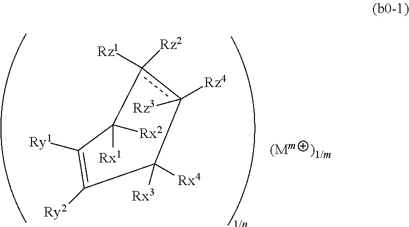
wherein Rx¹ to Rx⁴ each independently represents a hydrocarbon group which may have a substituent or a hydrogen atom, or two or more thereof may be bonded to each other to form a ring structure, Ry¹ and Ry² each independently represents a hydrocarbon group which may have a substituent or a hydrogen atom, or may be bonded to each other to form a ring structure, [symbol]
a doublet line of a dotted line and a straight line represents a double bond or a single bond, and where valence permits, Rz¹ to Rz⁴ each independently represents a hydrocarbon group which may have a substituent or a hydrogen atom, or two or more thereof may be bonded to each other to form a ring structure, where at least one of two or more of Rx¹ to Rx⁴, Ry¹ and Ry², and two or more of Rz¹ to Rz⁴ are bonded to each other to form an aromatic ring, at least one of Rx¹ to Rx⁴, Ry¹ and Ry², and Rz¹ to Rz⁴ contains an anion group represented by General Formula (b0-r-an1), an entire anion moiety is an n-valent anion, at least one of Rx¹ to Rx⁴, Ry¹ and Ry², and Rz¹ to Rz⁴ contains a hydrocarbon group having a bromine atom or a hydrocarbon group having an iodine atom, n represents an integer of 1 or greater, m represents an integer of 1 or greater, and M^{m+} represents an m-valent organic cation, 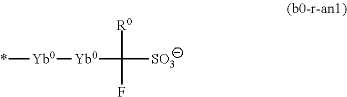
wherein Yb⁰ represents a divalent linking group or a single bond, Vb⁰ represents a single bond, an alkylene group, or a fluorinated alkylene group, R⁰ represents a fluorinated alkyl group having 1 to 5 carbon atoms or a fluorine atom, and * represents a bonding site.

4. The resist composition according to claim 1, wherein the hydrocarbon group having a bromine atom and the hydrocarbon group having an iodine atom are aromatic hydrocarbon groups.

5. A resist pattern forming method, comprising:
forming a resist film on a support using the resist composition according to claim 1;
exposing the resist film to light; and
developing the resist film exposed to light to form a resist pattern.

6. A compound represented by General Formula (b0), 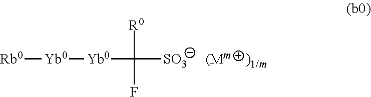
wherein Rb⁰ represents a fused cyclic group in which an aromatic ring and an alicyclic ring are fused, the alicyclic ring in the fused cyclic group has substituents, and at least one of the substituents contains a hydrocarbon group having a bromine atom or a hydrocarbon group having an iodine atom, Yb⁰ represents a divalent linking group or a single bond, wherein Yb⁰ is bonded to the alicyclic ring in the fused cyclic group, Vb⁰ represents a single bond, an alkylene group, or a fluorinated alkylene group, R⁰ represents a fluorinated alkyl group having 1 to 5 carbon atoms or a fluorine atom, M^{m+} represents an m-valent organic cation, and m represents an integer of 1 or greater.

7. An acid generator comprising the compound according to claim 6.
